(19) United States
(12) Patent Application Publication
(10) Pub. No.: US 2019/0083583 A1
(43) Pub. Date: Mar. 21, 2019

(54) ENZYME REPLACEMENT THERAPY IN MUCOPOLYSACCHARIDOSIS IIIB PATIENTS

(71) Applicant: ALEXION PHARMACEUTICALS, INC., New Haven, CT (US)

(72) Inventors: Sandra Rojas-Caro, Waltham, MA (US); Kiran C. Patki, Lexington, MA (US)

(21) Appl. No.: 16/073,529

(22) PCT Filed: Jan. 30, 2017
(86) PCT No.: PCT/US17/15646

§ 371 Date: Jul. 27, 2018

Related U.S. Application Data
(60) Provisional application No. 62/289,044, filed on Jan. 29, 2016.

(51) Int. Cl. A61K 38/47; A61K 9/00; A61P 3/00

(52) U.S. Cl. CPC A61K 38/47; A61P 3/00; C12Y 302/0105; A61K 9/0019

(57) ABSTRACT

This disclosure relates to method for treating Sanfilippo Syndrome B (also Mucopolysaccharidosis III B, MPSIIIB) by enzyme replacement therapy.

This application claims the benefit of U.S. Provisional Application Ser. No. 62/289,044, filed Jan. 29, 2016, which is incorporated herein by reference in its entirety.

### BACKGROUND

Sanfilippo Syndrome B (also known as Mucopolysaccharidosis III B, MPSIIIB) is an autosomal recessive lysosomal storage disease (LSD) caused by a deficiency in a lysosomal enzyme known as N-acetyl-alpha-D-glucosaminidase (NaGlu). NaGlu is required for the degradation of heparan sulfate as part of the stepwise breakdown of glycosaminoglycans (GAG) in the lysosome. The deficiency or absence of NaGlu leads to accumulation and urinary excretion of heparan sulfate. With over 70 different mutations identified to date, Sanfilippo Syndrome B exhibits extensive molecular and genetic heterogeneity.

Approximately 1 out of 200,000 births is affected by Sanfilippo Syndrome B and the deficiency mainly manifests in young children. After initial symptom-free interval, patients suffering from Sanfilippo Syndrome B usually present with a slowing of mental development and behavioral problems, followed by progressive intellectual decline resulting in severe mental retardation, dementia and motor disease. Acquisition of speech is slow and incomplete. Profoundly affected patients may present delayed psychomotor and speech development as early as 2 years of age. The disease usually progresses to increasing behavioral disturbance and sleep disturbance. Although the clinical features are mainly neurological, patients often develop diarrhea, carious teeth, an enlarged liver and spleen, stiff joints, hirsuteness and/or coarse hair and may exhibit blood-clotting problems. In the final stage of the illness, patients become immobile and unresponsive and develop swallowing difficulties and seizure. The life-span of an affected child typically does not extend beyond late teens to early twenties.

Different approaches have been attempted to provide the missing enzyme in patients. To produce NaGlu for enzyme replacement therapy (ERT), human NaGlu has been expressed in various mammalian cell culture systems. However, in contrast to the naturally occurring NaGlu which trafficks to the lysosome intracellularly, recombinant NaGlu proteins produced and secreted from mammalian cells were found to contain no or only a trace amount of mannose-6-phosphate (M6P). The absence or scarcity of M6P moieties in the secreted NaGlu has been known to prevent its efficient internalization into target cells (e.g., human skin fibroblasts), which have M6P receptors on the surface on its plasma membrane (see, Zhao et al., Protein Expression and Purification, 19:202-211 (2000); and Weber et al., Protein Expression and Purification, 21:251-259 (2001)). The low degree of phosphorylation was seen in secreted mouse NaGlu expressed in CHO cells, secreted human NaGlu expressed in HeLa cells, secreted human NaGlu expressed in human fibroblasts, and secreted human NaGlu expressed in human embryonic kidney (HEK) cell line 293 (see, Zhao et al., Protein Expression and Purification, 19:202-211 (2000); Yogalingam et al., Biochim Biophys. Acta 1502: 415-425; and Weber et al., Protein Expression and Purification, 21:251-259 (2001)). No or weak phosphorylation of N-glycans in the NaGlu proteins secreted from the mammalian cells has posed a major obstacle for the development of a recombinant human NaGlu protein suitable for enzyme replacement therapy, as all the aforementioned attempts have failed to produce an enzyme which is efficiently taken up by target cells as the concentration of the internalized proteins, if detectable at all, was nearly a thousand times less than wild-type levels (see, Zhao et al., Protein Expression and Purification, 19:202-211 (2000)).

### SUMMARY

This disclosure relates to method for treating Sanfilippo Syndrome B (also known as Mucopolysaccharidosis III B, MPSIIIB) by enzyme replacement therapy. In some embodiments, the methods involve the intravenous administration of an effective amount of human NaGlu protein, preferably recombinant human NaGlu protein (rhNaGlu), to a subject in need thereof every other week (QOW). In some embodiments, the method includes administering a dose of about 0.3 mg/kg to about 10 mg/kg of rhNaGlu every other week. For example, a dose of about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg, can be administered every other week. Preferably, the intravenous administration is by intravenous infusion, such as a two hour intravenous infusion.

As described and exemplified herein, intravenous administration of human NaGlu protein in accordance with the methods resulted in a decrease in total heparan sulfate levels in the cerebral spinal fluid in MPSIIIB patients in comparison to base line levels before administration. In addition, intravenous administration of human NaGlu in accordance with the methods also stabilized or improved brain morphomety (e.g., cortical gray matter volume) and neurocognitive scores (e.g., Bayley Scales of Infant Development Third Edition (BSID-III) and Kaufman Assessment Battery for Children Second Edition (KABC-II)).

In one aspect, the disclosure provides a method for treating a subject suffering from N-acetyl-alpha-D-glucosaminidase (NaGlu) deficiency, wherein a therapeutically effective amount of recombinant human N-acetyl-alpha-D-glucosaminidase (rhNaGlu) is administered to the subject in a manner and/or according to a dosage regimen described herein. In another aspect, the disclosure provides recombinant human N-acetyl-alpha-D-glucosaminidase (rhNaGlu) for use in treating N-acetyl-alpha-D-glucosaminidase (NaGlu) deficiency, wherein the rhNaGlu is administered in a manner and/or according to a dosage regime as described herein. In another aspect, the disclosure provides a use of recombinant human N-acetyl-alpha-D-glucosaminidase (rhNaGlu) for the manufacture of a medicament for treating N-acetyl-alpha-D-glucosaminidase (NaGlu) deficiency, wherein the medicament is administered, or prepared to be administered, in a manner and/or according to a dosage regimen as described herein. In some embodiments, the NaGlu deficiency from which the subject is suffering is mucopolysaccharidosis IIIB (MPS IIIB) (Sanfilippo syndrome B).

The method and manner of administration includes periodically administering a therapeutically effective amount of recombinant human N-acetyl-alpha-D-glucosaminidase (rhNaGlu) to a subject intravenously, for example via intravenous infusion or intravenous injection. A preferred mode of administration is intravenous infusion.

In some embodiments, rhNaGlu includes a sufficient amount of mannose-6-phosphate (M6P) or bis-M6P for internalization into a cell of the subject. For example, M6P can be present at a concentration that is greater than about 0.5 mole per mole of protein. In some embodiments, rhNaGlu contains fewer than five sialic acid residues per protein.

A therapeutically effective amount of rhNaGlu as described herein can produce one or more positive outcomes in a subject to whom it is administered. For example, a therapeutically effective amount of rhNaGlu can be effective to slow, arrest, or reverse the progression of mucopolysaccharidosis IIIB (MPS IIIB) in a subject; and/or to slow, arrest, or reverse neurocognitive decline in a subject; and/or to improve one or more neurocognitive or behavioral indicators for a subject; and/or to slow a decline in, stabilize, or increase the cortical gray matter volume of the brain of a subject; and/or to reducing the total heparan sulfate level in cerebral spinal fluid (CSF) of a subject, without limitation. Thus, the method of treatment provided by the disclosure can include a method of slowing, arresting or reversing the progression of mucopolysaccharidosis IIIB (MPS IIIB) in a subject; a method of slowing, arresting, or reversing neurocognitive decline in a subject suffering from NaGlu deficiency; a method for improving at least one neurocognitive and/or behavioral indicator for subject suffering from NaGlu deficiency; a method for slowing a decline in, stabilizing, or increasing the cortical gray matter volume of the brain of a subject suffering from NaGlu deficiency; and a method for reducing the total heparan sulfate level in cerebral spinal fluid (CSF) of a subject suffering from NaGlu deficiency.

In some embodiments, rhNaGlu is administered to a subject in a multiplicity of doses over a period of time. For example, a dose of rhNaGlu can be administered to a subject once per week, once every two weeks, once every three weeks, once every four weeks, or at other frequencies suitable for treatment, as described herein. As another example, rhNaGlu can be administered to a subject at least once per week, at least once every two weeks, at least once every three weeks, or at least once every four weeks. The frequency of administration, or interval between doses, can, but need not, change over time during a course of treatment. The period of time over which the multiplicity of doses of rhNaGlu is administered, that is, the duration of treatment, can be a fixed period or it can be an indefinite period. For example, rhNaGlu can be administered to a subject for a period of any number of weeks, such as 12 weeks, 24 weeks, 40 weeks, 52 weeks or longer. As another example, rhNaGlu can be administered to a subject for a period of at least 12 weeks, for a period of at least 24 weeks, for a period of at least 40 weeks, or for a period of at least 52 weeks or longer. As another example, rhNaGlu can be administered until a specified or predetermined treatment goal or endpoint is reached. Alternatively, rhNaGlu can be administered for the duration of the subject's life. Administration of the multiplicity of doses of rhNaGlu is typically accomplished by way of intravenous infusion, but other modes of administration as described herein are also contemplated.

In some embodiments, the dose of rhNaGlu administered to a subject is about 0.3 milligram (mg) of rhNaGlu per kilogram (Kg) of body weight (0.3 mg/Kg), about 1 mg/Kg, about 3 mg/Kg, about 5 mg/Kg, about 10 mg/Kg, about 15 mg/Kg, about 20 mg/Kg, about 25 mg/Kg, about 30 mg/Kg, about 35 mg/Kg, about 40 mg/Kg, or higher. In some embodiments, the dose of rhNaGlu administered to a subject is at least about 0.3 mg/Kg, at least about 1 mg/Kg, at least about 3 mg/Kg, at least about 5 mg/Kg, at least about 10 mg/Kg, at least about 15 mg/Kg, at least about 20 mg/Kg, at least about 25 mg/Kg, at least about 30 mg/Kg, at least about 35 mg/Kg, at least about 40 mg/Kg, or higher. In some embodiments, the dose of rhNaGlu administered to a subject is between about 0.3 mg/Kg to about 10 mg/Kg, or between about 3 mg/Kg to about 10 mg/Kg, or between about 5 mg/Kg to about 10 mg/Kg. Dosages levels recited here and elsewhere in the disclosure are to be understood to be both generally and specifically applicable to the treatment methods and regimens described herein. The amount of rhNaGlu administered to a subject per dose of rhNaGlu can, but need not, change over time during a course of treatment. Administration of rhNaGlu is typically accomplished by way of intravenous infusion, but other modes of administration as described herein are also contemplated.

In some embodiments that include administration of a multiplicity of doses of rhNaGlu, a first period of treatment is followed by dose escalation for a second period of treatment. In some embodiments, dose escalation takes the form of an increase in the amount of rhNaGlu administered per dose (i.e., a higher dose of rhNaGlu). A first amount of rhNaGlu can be administered as a first dose periodically for a first period, followed by a second, higher amount of rhNaGlu administered as a second dose periodically for a second period. In other embodiments, dose escalation takes the form of an increase in frequency of dose administration. In some embodiments, dose escalation includes both higher and more frequent doses of rhNaGlu. For example, the higher dose of rhNaGlu can be about 5 mg/Kg, about 10 mg/Kg, about 15 mg/Kg, or higher; or it can be at least about 5 mg/Kg, at least about 10 mg/Kg, at least about 15 mg/Kg, or higher. Also contemplated by the disclosure is a first period of treatment followed by a dose de-escalation for a second period. Administration of rhNaGlu is typically accomplished by way of intravenous infusion, but other modes of administration as described herein are also contemplated.

In some embodiments, the dose of rhNaGlu is sufficient to generate a therapeutically effective serum concentration of rhNaGlu. The serum concentration of rhNaGlu can be, for example, greater than 4000 ng/mL, or greater than 15,000 ng/mL. In some embodiments, multiple doses of rhNaGlu are administered to the subject to yield pharmacokinetic exposure that is more than dose-proportional.

In some embodiments, a subject to whom rhNaGlu is administered in accordance with the methods described herein is under 18 years of age. For example, the chronological age of a subject to whom rhNaGlu is administered can be 1, 2, 3, 4, 5, 6, 7, or 8 years of age, or older than 8 years of age. As another example, the chronological age of a subject to whom rhNaGlu is administered can be under 1, under 2, under 3, under 4, under 5, under 6, under 7, or under 8 years of age, or older than 8 years of age. A subject to whom rhNaGlu is administered may be exhibit neurocognitive impairment prior to initiation or escalation of treatment. For example, the cognitive developmental quotient (DQ) of a subject at the time treatment is initiated or escalated can be under 100 but at least 50, or at least 60, or at least 70, or at least 80, or at least 90.

Cortical gray matter volume in subjects suffering from N-acetyl-alpha-D-glucosaminidase (NaGlu) deficiency may decline over time, or may not increase at a normal rate. In some embodiments, rhNaGlu is administered in an amount and for a period sufficient to slow a decline in, or to stabilize, or to increase the cortical gray matter volume of the brain of the subject, in comparison to a baseline level of cortical gray matter volume of the subject determined prior to initiation or escalation of treatment. A baseline level for the subject for any desired measurement can be evaluated prior to or at the time of initiation of treatment, or prior to or at the time of an escalation of treatment. The disclosure thus also contemplates a method for slowing a decline in, stabilizing, or increasing the cortical gray matter volume of the brain of a subject suffering from NaGlu deficiency, wherein a therapeutically effective amount of rhNaGlu is periodically intravenously administering to the subject. In an exemplary embodiment, rhNaGlu is administered to a subject for a period of 24 weeks or at least 24 weeks, and cortical gray matter volume of the subject is unchanged or increases in comparison to the baseline level. An increase in cortical gray matter volume can be 1%, 2%, 3%, 4%, 5%, 10%, 15%, 20% or more over the baseline level of cortical gray matter volume. In another embodiment, rhNaGlu is administered to the subject for a period of 52 weeks or at least 52 weeks, and cortical gray matter volume of the subject is at least 70%, 75%, 80%, 85%, 90%, 91%, 92%, 93%, 94%, 95%, 96%, 97%, 98%, 99% or 100% or more of the baseline level of cortical gray matter volume. In some embodiments, the chronological age of the subject at the time treatment is initiated or escalated is 0 months, 3 months, 6 months, 9 months, 12 months, 18 months, 24 months, 30 months, 36 months, 42 months, 48 months, 60 months, 72 months, 84 months, 96 months, 108 months, 120 months, or higher. In some embodiments, the chronological age of the subject at the time treatment is initiated or escalated is less than or equal to 3 months, 6 months, 9 months, 12 months, 18 months, 24 months, 30 months, 36 months, 42 months, 48 months, 60 months, 72 months, 84 months, 96 months, 108 months, 120 months, or higher. In some embodiments, the chronological age of the subject at the time treatment is initiated or escalated is greater than 0 months, 3 months, 6 months, 9 months, 12 months, 18 months, 24 months, 30 months, 36 months, 42 months, 48 months, 60 months, 72 months, 84 months, 96 months, 108 months, 120 months, or higher. In some embodiments, the chronological age of the subject is between two ages, such as, for example, between 18 and 30 months, or between 24 and 40 months, or between 36 and 60 months.

In some embodiments, the comparison of cortical gray matter volume is made against the subject's own baseline value; however, in other embodiments, the subject's cortical gray matter volume after treatment has been initiated or escalated is compared to the cortical gray matter volume of an untreated age-matched patient (or a plurality of age-matched patients, in which case an average value can be used) suffering from mucopolysaccharidosis IIIA (MPSIIIA) or mucopolysaccharidosis IIIB (MPSIIIB). A cortical gray matter volume after treatment that is greater than the cortical gray matter volume of the age-matched patient(s) indicates a positive treatment outcome. An “age-matched” patient can be a patient who is the same age as the subject, or whose age is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more months of the age of the subject. An “age-matched” patient can be younger or older than the subject, and may encompass a broader range of ages for an older subject. For example, in some embodiments of the method of treatment of a subject under about 36 months, an age-matched patient may have an age within 12 months of the subject; in some embodiments of the method of treatment of a subject over about 36 months of age, an age-matched patient may have an age within 12 months or within 24 months of the subject; in some embodiments of the method of treatment of a subject over about 72 months of age, an age-matched patient may have an age within 12 months, within 24 months, within 36 months, within 48 months or more of a subject.

Optionally, the method of treatment in which rhNaGlu is administered in an amount and for a period sufficient to slow a decline in, or to stabilize, or to increase the cortical gray matter volume of the brain of the subject also includes determining the cortical gray matter volume of the subject. The cortical gray matter volume of the subject can be determined at any time during treatment or at the conclusion of treatment. For example, the cortical gray matter volume can be determined by subjecting the subject to structural magnetic resonance imaging (MRI).

Neurocognitive development in subjects suffering from N-acetyl-alpha-D-glucosaminidase (NaGlu) deficiency may stall or regress, or may not proceed at a normal rate. In some embodiments, rhNaGlu is administered in an amount and for a period sufficient to slow a decline in, stabilize, or improve a neurocognitive indicator of the subject in comparison to a baseline level of the neurocognitive indicator of the subject determined prior or at the time of initiation or escalation of treatment. The disclosure thus also contemplates a method of slowing, arresting, or reversing neurocognitive decline, or for improving a neurocognitive indicator, in a subject suffering from NaGlu deficiency, wherein a therapeutically effective amount of rhNaGlu is periodically intravenously administering to the subject. Improvement in a neurocognitive indicator may be indicated by improvement over a baseline value for the indicator for the subject, or improvement compared a corresponding neurocognitive indicator for an untreated age-matched patient (or a plurality of age-matched patients, in which case an average value can be used) suffering from mucopolysaccharidosis IIIA (MPSIIIA) or mucopolysaccharidosis IIIB (MPSIIIB). In some embodiments, the chronological age of the subject at the time treatment is initiated or escalated is, or is at least, 0 months, 3 months, 6 months, 9 months, 12 months, 18 months, 24 months, 30 months, 36 months, 42 months, 48 months, 60 months, 72 months, 84 months, 96 months, 108 months, 120 months, or higher. In some embodiments, the chronological age of the subject is between two ages. The chronological age of the subject can be, for example, less than or equal to 36 months, between 18 and 30 months, between 24 and 40 months, between 36 and 60 months, or greater than 60 months.

The neurocognitive indicator can be measured using a neurocognitive assessment instrument such as, for example, the Bayley Scales of Infant Development-Third Edition (BSID-III) and/or the Kaufman Assessment Battery for Children-Second Edition (KABC-II). A neurocognitive assessment instrument can be used to determine an indicator such as cognitive age equivalence (AEq) or cognitive developmental quotient (DQ).

In some embodiments, rhNaGlu is administered to a subject for a period of 12 weeks, 24 weeks, 40 weeks, 52 weeks, or longer, or for a period of at least 12 weeks, at least 24 weeks, at least 40 weeks, at least 52 weeks, or longer, and the cognitive age equivalence (AEq) of the subject is unchanged or increases in comparison to the baseline level. An increase relative to the baseline level of the subject can be 1 month, 2 months, 3 months, 4 months, 5 months, 6 months, 7 months, 8 months, 9 months, 10 months, 11 months, 12 months, or more. In some embodiments, the chronological age of the subject at the time treatment is initiated or escalated is less than or equal to 3 months, 6 months, 9 months, 12 months, 18 months, 24 months, 30 months, 36 months, 42 months, 48 months, 60 months, 72 months, 84 months, 96 months, 108 months, 120 months, or higher. In some embodiments, rhNaGlu is administered to a subject for a period of 12 weeks, 24 weeks, 40 weeks, 52 weeks, or longer, or for a period of at least 12 weeks, at least 24 weeks, at least 40 weeks, at least 52 weeks, or longer, and the cognitive age equivalence (AEq) of the subject lower than the baseline level. The cognitive age equivalence (AEq) can by lower than the baseline level by 1 month, 2 months, 3 months, 4 months, 5 months, 6 months or more. In one embodiment, the cognitive age equivalence (AEq) of the subject after treatment for the specified period is no less than 3 months lower than the baseline level (for example, it may be 3 months lower than the baseline level, 2 months lower than the baseline level, or one month lower than the baseline level).

In some embodiments, rhNaGlu is administered to a subject for a period of 12 weeks, 24 weeks, 40 weeks, 52 weeks, or longer, or for a period of at least 12 weeks, at least 24 weeks, at least 40 weeks, at least 52 weeks, or longer, and the cognitive developmental quotient (DQ) of the subject is increased, remains unchanged, or is reduced by no more than 15 points, reduced by no more than 10 points, or reduced by no more than 5 points. For example, rhNaGlu can be administered to the subject for a period of at least 24 weeks or at least 52 weeks, and the cognitive development quotient (DQ) of the subject is no lower than 15 points, or no lower than 10 points, below the baseline level.

Behavioral development in subjects suffering from N-acetyl-alpha-D-glucosaminidase (NaGlu) deficiency may stall or regress, or may not proceed at a normal rate. In some embodiments, rhNaGlu is administered in an amount and for a period sufficient to slow a decline in, stabilize, or improve a behavioral indicator of the subject in comparison to a baseline level of the behavioral indicator of the subject determined prior or at the time of initiation or escalation of treatment. The disclosure thus also contemplates a method for improving a behavioral indicator in a subject suffering from NaGlu deficiency, wherein a therapeutically effective amount of rhNaGlu is periodically intravenously administering to the subject. Improvement in a behavioral indicator may be indicated by improvement over a baseline value for the indicator for the subject, or improvement compared a corresponding behavioral indicator for an untreated age-matched patient suffering from mucopolysaccharidosis IIIA (MPSIIIA) or mucopolysaccharidosis IIIB (MPSIIIB). In some embodiments, the chronological age of the subject at the time treatment is initiated or escalated is, or is at least, 0 months, 3 months, 6 months, 9 months, 12 months, 18 months, 24 months, 30 months, 36 months, 42 months, 48 months, 60 months, 72 months, 84 months, 96 months, 108 months, 120 months, or higher. In some embodiments, the chronological age of the subject is between two ages, such as, for example, between 18 and 30 months, or between 24 and 40 months, or between 36 and 60 months.

The behavioral indicator can be measured using a behavioral assessment instrument such as, for example, the Vineland Adaptive Behavior Scales—Second Edition (VABS-II), which can be administered with or without motor skills assessment. A behavioral assessment instrument can be used to determine an indicator such as behavioral age equivalence (AEq) or behavioral developmental quotient (DQ).

In some embodiments, rhNaGlu is administered to a subject for a period of 12 weeks, 24 weeks, 40 weeks, 52 weeks, or longer, or for a period of at least 12 weeks, at least 24 weeks, at least 40 weeks, at least 52 weeks, or longer. The behavioral age equivalence (AEq) of the subject can be unchanged or increased in comparison to the baseline level. In an exemplary embodiment wherein the chronological age of the subject at baseline is less than or equal to 36 months, the behavioral age equivalence (AEq) of the subject may be unchanged or increased in comparison to the baseline level, or it may be lower than the baseline level. The behavioral age equivalence (AEq) can by lower than the baseline level by 1 month, 2 months, 3 months, 4 months, 5 months, 6 months or more. In an exemplary embodiment wherein the chronological age of the subject at baseline is greater than 36 months, the behavioral age equivalence (AEq) of the subject can be lower than the baseline level; for example, it can be no less than 15 months lower than the baseline level, no less than 10 months lower than the baseline level, or no less than 5 months lower than the baseline level. In some embodiments, rhNaGlu is administered to a subject for a period of 12 weeks, 24 weeks, 40 weeks, 52 weeks, or longer, or for a period of at least 12 weeks, at least 24 weeks, at least 40 weeks, at least 52 weeks, or longer, and the behavioral developmental quotient (DQ) of the subject is increased or remains unchanged. In some embodiments wherein the chronological age of the subject at baseline is less than or equal to 60 months, the behavioral development quotient (DQ) of the subject decreases by no more than 40 points from the baseline level, or nor more than 30 points from the baseline level, or no more than 20 points from the baseline level, or no more than 10 points from the baseline level. In some embodiments wherein the chronological age of the subject at baseline is greater than 60 months, the behavioral development quotient (DQ) of the subject decreases by no more than 20 points from the baseline level, or no more than 10 points from the baseline level.

In some embodiments, the value of the subject's neurocognitive indicator or behavioral indicator is compared against the subject's own baseline value for the indicator; however, in other embodiments, the value of the subject's neurocognitive indicator or behavioral indicator after treatment has been initiated or escalated is compared to the value of a corresponding neurocognitive indicator or behavioral indicator of an untreated age-matched patient (or a plurality of age-matched patients, in which case an average value can be used) suffering from mucopolysaccharidosis IIIA (MPSIIIA) or mucopolysaccharidosis IIIB (MPSIIIB).

Optionally, the method of treatment in which rhNaGlu is administered in an amount and for a period sufficient to slow a decline in, stabilize, or improve a neurocognitive and/or behavioral indicator of the subject also includes assessing the neurocognitive or behavioral indicator of the subject. The neurocognitive or behavioral indicator of the subject can be assessed by administering an assessment instrument, such as the BSID-III, the KABC-II, and/or the VABS-II, to the subject.

Heparan sulfate may accumulate in the cerebral spinal fluid (CSF) of subjects suffering from N-acetyl-alpha-D-glucosaminidase (NaGlu) deficiency. In some embodiments, rhNaGlu is administered in an amount and for a period sufficient to decrease the total heparan sulfate (HS) level in the cerebral spinal fluid (CSF) of the subject in comparison to a baseline level of the subject determined prior to initiation or escalation of treatment. The disclosure thus also contemplates a method for reducing the total heparan sulfate (HS) level in cerebral spinal fluid (CSF) of a subject suffering from NaGlu deficiency, wherein a therapeutically effective amount of rhNaGlu is periodically intravenously administering to the subject. In some embodiments, rhNaGlu is administered in an amount and for a period sufficient to reduce total HS level in the CSF of the subject to less than about 95%, 90%, 85%, 80%, 75%, 70%, 65%, 60%, 55%, 50%, 45%, 40%, 35%, 30% or smaller percentage of the baseline level. For example, rhNaGlu can be administered to the subject for a period of at least 12 weeks, and the total HS level in the CSF of the subject is reduced to less than 90% of the baseline level. As another example, rhNaGlu can be administered to the subject for a period of at least 24 weeks, and the total HS level in the CSF of the subject is reduced to less than 75% of the baseline level. As another example, rhNaGlu can be administered to the subject for a period of at least 42 weeks, and the total HS level in the CSF of the subject is reduced to less than 85% of the baseline level. Optionally, the method also includes determining the total heparan sulfate level in the CSF of the subject. The total HS level can be determined at any time during treatment or at the conclusion of treatment.

The disclosure provides recombinant human N-acetyl-alpha-D-glucosaminidase (rhNaGlu) for use in treating N-acetyl-alpha-D-glucosaminidase (NaGlu) deficiency, e.g., MPS IIIB, wherein rhNaGlu is administered by intravenous infusion in a manner and/or according to a dosage regime as described herein. The disclosure also provides a use of recombinant human N-acetyl-alpha-D-glucosaminidase (rhNaGlu) for the manufacture of a medicament for treating N-acetyl-alpha-D-glucosaminidase (NaGlu) deficiency, wherein the medicament is administered, or prepared to be administered, by intravenous infusion in a manner and/or according to a dosage regime as described herein. For example, rhNaGlu can be administered by intravenous infusion once every three weeks or at least once every three weeks, or once every two weeks or at least once every two weeks, in an amount of about 1 mg/Kg, about 3 mg/Kg, about 5 mg/Kg, or about 10 mg/Kg, or in amount of at least about 1 mg/Kg, at least about 3 mg/Kg, at least about 5 mg/Kg, or at least about 10 mg/Kg, for 24 weeks, 40 weeks, or 52 weeks, or for at least 24 weeks, at least 40 weeks, or at least 52 weeks. The dosage regime can include a dose escalation as described in more detail elsewhere herein. For example, rhNaGlu can be administered, or prepared to be administered, at dose of less than 5 mg/Kg for at least 24 weeks, followed by a dose of at least 5 mg/Kg for at least 24 weeks.

Also provided by the disclosure is a pharmaceutical composition that includes recombinant human NaGlu (rhNaGlu) and a pharmaceutical carrier, formulated for intravenous infusion. The rhNaGlu is present in an amount sufficient to stabilize or increase the cortical gray matter volume of the brain of a subject, and/or stabilize or improve a neurocognitive indicator of the subject, and/or decrease the level of heparan sulfate in the cerebral spinal fluid of a subject, in comparison to a baseline level of the subject determined prior to initiation or escalation of treatment. In some embodiments, thepharmaceutical composition contains an effective amount of human NaGlu for intravenous administration to a subject in need thereof every other week (QOW).

### BRIEF DESCRIPTION OF THE FIGURES

FIG. 1 depicts the amino acid sequence of human recombinant NaGlu (amino acid residues 1-23, signal peptide, SEQ ID NO:1). See, e.g., patent publications WO2013/055888; US2013/0095092, and US2014/0255383;.UniProtKB P54802 ANAG_Human shows the corresponding wild-type human NaGlu protein sequence.

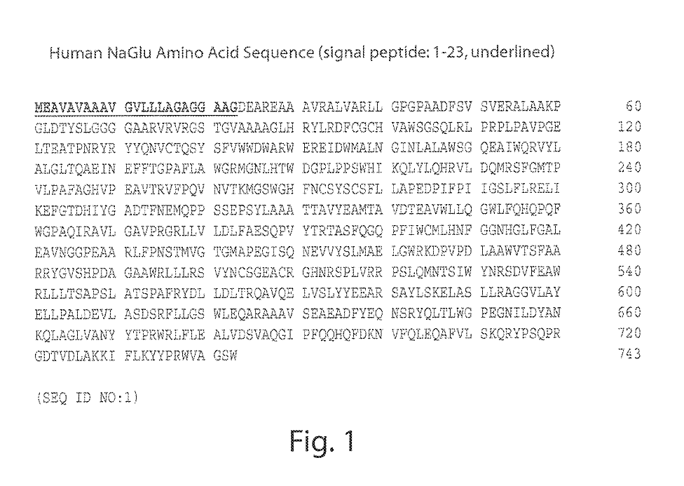

FIG. 2A depicts the nucleic acid sequence (cDNA) of human recombinant NaGlu (SEQ ID NO:2), including the nucleic acid sequence encoding the signal peptide.

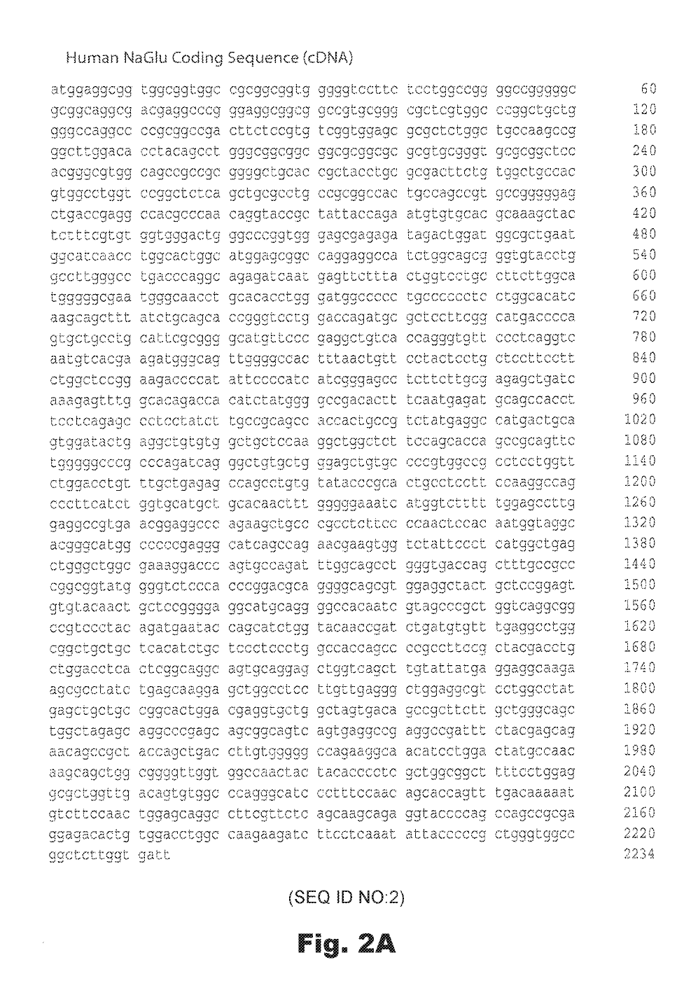

FIG. 2B depicts cDNA of wild-type human NaGlu (SEQ ID NO:5), as determined from the mRNA sequence reported at NCBI Reference Sequence: NM_000263.3.

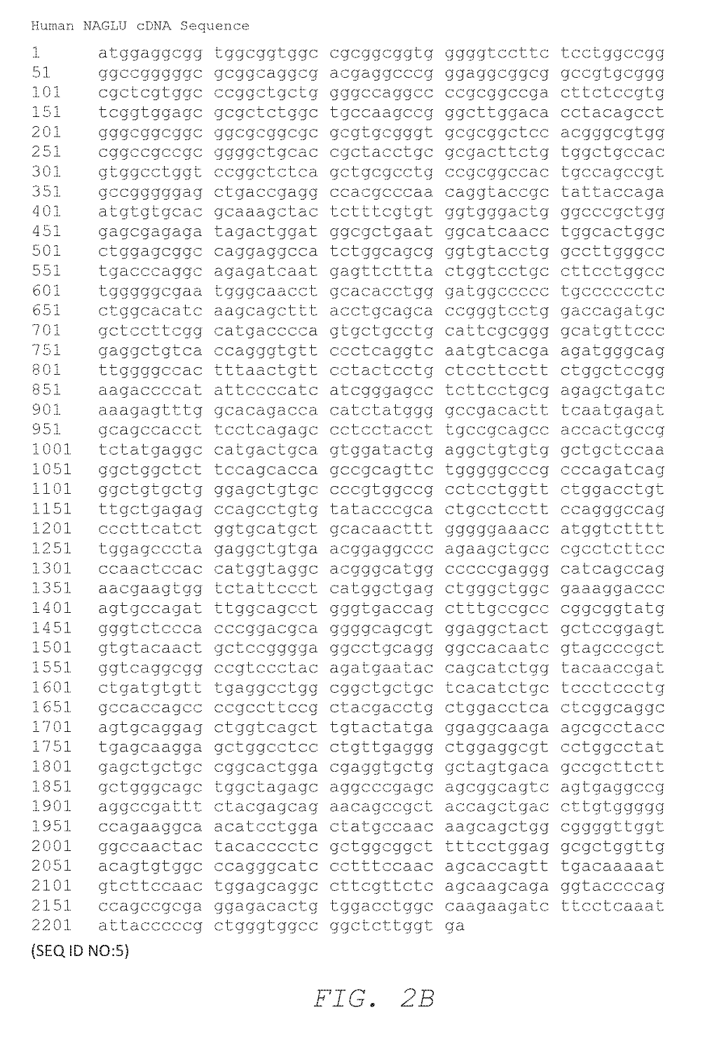

FIG. 3 depicts the nucleic acid sequence of 1.1 kb ovalbumin promote (SEQ ID NO:3)r.

FIG. 4A-FIG. 4D depict the nucleic acid sequence of pSIN-OV-1.1-I-rhNaGlu vector (SEQ ID NO:4) used in transgenesis of an avian.

FIG. 5 is a schematic representation of pSIN-OV-1.1-I-rhNaGlu vector.

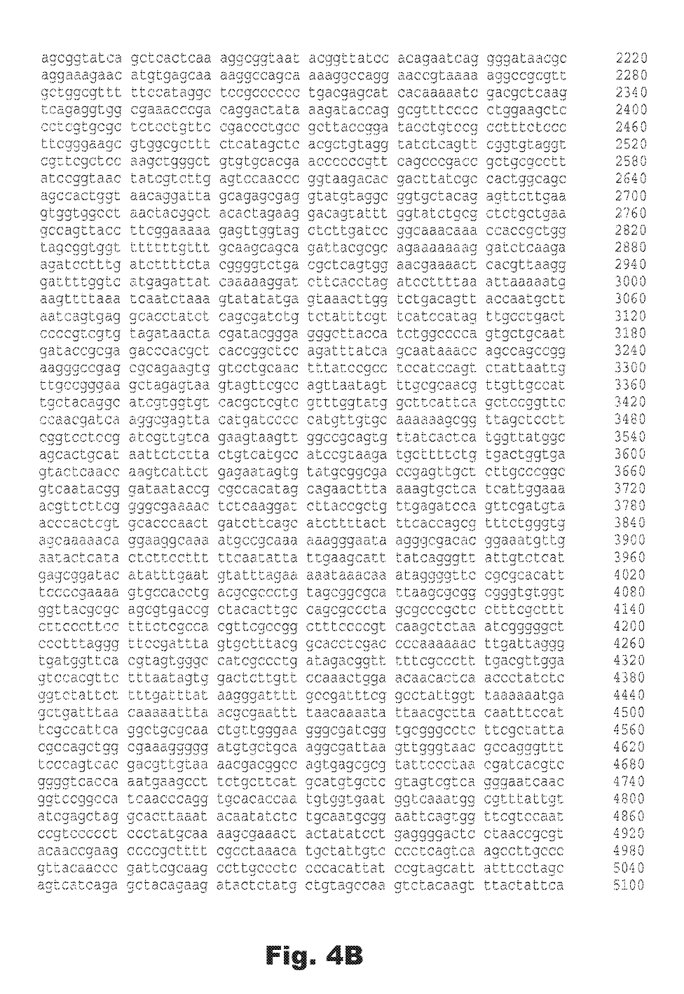

FIG. 6 depicts Western analysis of rhNaGlu isolated and purified from egg white of a transgenic Gallus.

FIG. 7 depicts the average concentration of rhNaGlu deposited in egg white of transgenic Gallus.

FIG. 8 depicts an oligosaccharide profile of rhNaGlu produced from a transgenic Gallus using HPAEC-PAD.

FIG. 9 depicts uptake analysis of rhNaGlu by human skin fibroblasts (MPS IIIB, NaGlu deficient; Normal, wild-type human skin fibroblast; 1U of enzymatic activity=nmol of protein/hr).

FIG. 10 depicts uptake inhibition analysis of rhNaGlu (Gallus) using various concentrations of M6P monosaccharide (1U of enzymatic activity=1 μmol of protein/min).

FIG. 11 depicts a schematic representation of pTT22 vector containing a recombinant human NaGlu fusion construct (AAA-NaGlu: acidic amino acid residues fused to N-terminus of the full length NaGlu).

FIG. 12 depicts a schematic representation of pTT22 vector containing a recombinant human NaGlu fusion construct (NaGlu-TfRL: transferrin receptor ligand fused to C-terminus of the full length NaGlu).

FIG. 13 depicts enzymatic activity of AAA-NaGlu produced from HEK293 as compared to rhNaGlu produced from Gallus.

FIG. 14 depicts enzymatic activity of NaGlu-TfRL produced from HEK293 as compared to AAA-NaGlu produced from HEK293.

FIG. 15 depicts uptake levels of rhNaGlu (Gallus) into a macrophage cell line (NR8383) over time (48 hours). Cellular NaGlu activity was measured in units/mg of protein.

FIG. 16 depicts heparan sulfate substrate levels (μg/mg tissue) in the kidney of naglu (^{-/-}) mice following intravenous administration of vehicle (KO); rhNaGlu gallus at a dosage concentration of 6.25 mg/kg; or rhNaGlu gallus at a dosage concentration of 27 mg/kg. Wild type (WT) mice were untreated.

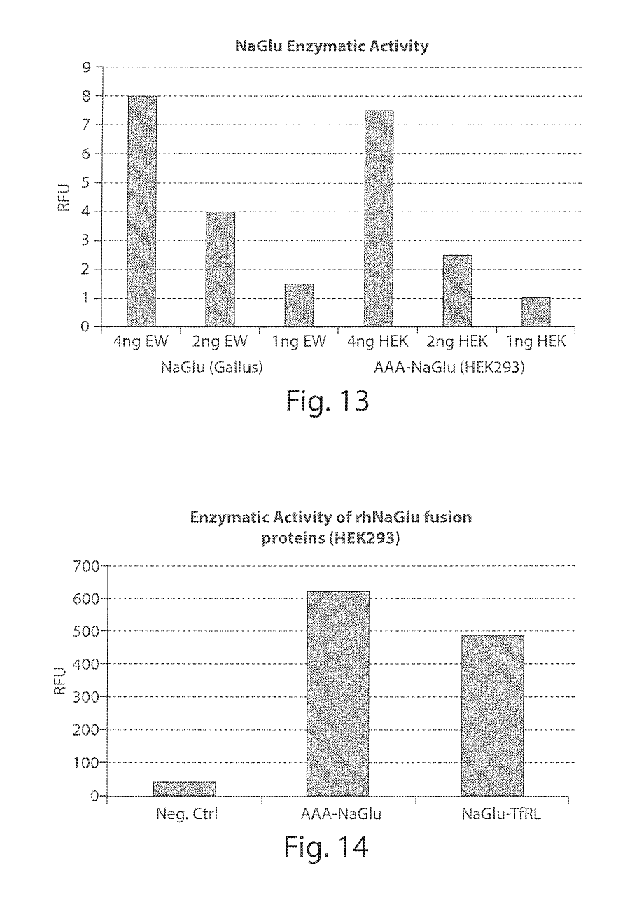

FIG. 17 depicts heparan sulfate substrate levels (μg/mg tissue) in the brain of naglu (^{-/-}) mice following intravenous administration of vehicle (KO); rhNaGlu gallus at a dosage concentration 6.25 mg/kg; or rhNaGlu gallus at a dosage concentration of 27 mg/kg. Wild type (WT) mice were untreated.

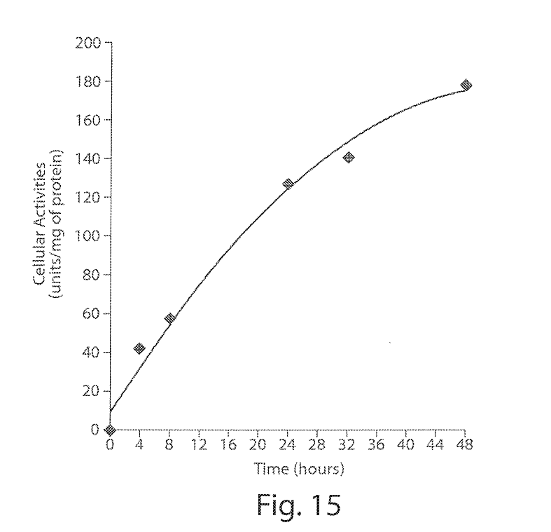

FIG. 18 depicts heparan sulfate substrate levels (μg/mg tissue) in the liver of naglu (^{-/-}) mice following intravenous administration of vehicle (KO); rhNaGlu gallus at a dosage concentration of 6.25 mg/kg; or rhNaGlu gallus at a dosage concentration of 27 mg/kg. Wild type (WT) mice were untreated.

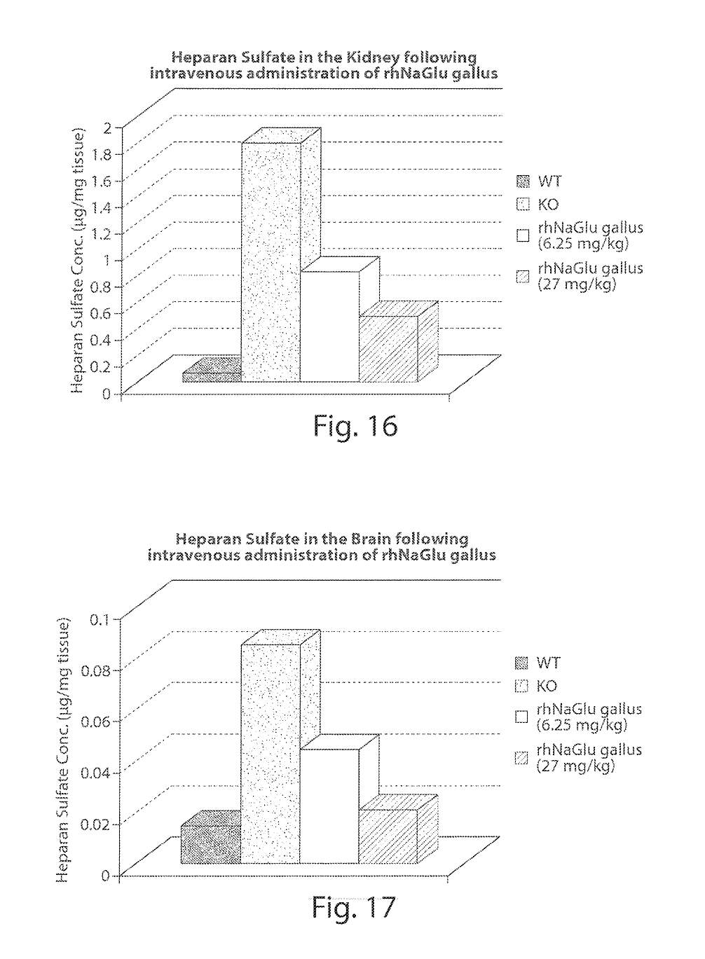

FIG. 19 depicts heparan sulfate substrate levels (μg/mg tissue) in the brain of naglu (^{-/-}) mice following intrathecal administration of vehicle (KO) or rhNaGlu gallus at a dosage concentration of 0.31 mg/kg. Wild type (WT) mice were untreated.

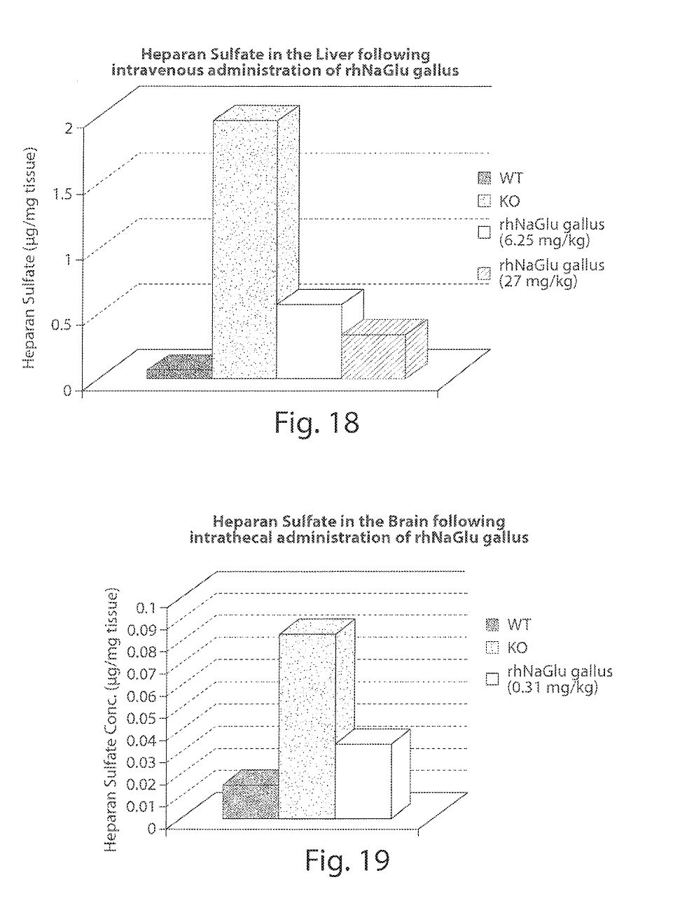

FIG. 20 is a graph showing levels of rhNaGlu in serum and CSF after IV administration at 3 mg/kg/dose.

FIG. 21 is a semi-log plot of rhNaGlu levels in serum (diamonds) or CSF (squares) following a single 1 hour IV infusion at 30 mg/kg/dose monitored over 9 hours. Each time point represents the average of 6 samples tested: 3 animals×2 weekly doses. Pre-dose rhNaGlu concentrations were subtracted from each CSF time point before plotting on graph.

FIG. 22 is a schematic showing the design of the clinical study of Example 9.

FIG. 23A depicts mean (+SEM) SBC-103 (rhNaGlu) serum concentration-time profiles for administration of rhNaGlu in the amounts of 0.3 mg/kg, 1.0 mg/kg, 3.0 mg/kg (day 0, week 12 and week 24); as well as 5.0 mg/kg and 10 mg/kg (day 0 for Part C of the clinical study).

FIG. 23B shows a comparison of pharmacokinetic profiles at Part C-Day 0, with pharmacokinetic profiles in Part A of the clinical study.

FIG. 24A shows the linear correlation of Cₘₐₓ and AUCₗₐₛₜ (AUC=area under the curve) with rhNaGlu dose for dose levels of 0.3 to 3.0 mg/kg at day 0, week 12 and week 24.

FIG. 24B shows increases in pharmacokinetic exposures as a function of rhNaGlu dose.

FIG. 25 shows the relationship between the pharmacodynamic parameter, reduction in total heparan sulfate in CSF, and the pharmacokinetic parameters, Cₘₐₓ and AUCₐₗₗ, at weeks 12 and 24 in of the clinical study. The dotted line represents zero, and vertical lines represent the mean values+/−standard deviation.

FIG. 26A shows cortical gray matter volume for individual subjects at baseline, week 24, and Part C baseline following administration of rhNaGlu.

FIG. 26B shows cortical gray matter volume vs. chronological age for individual subjects at baseline, week 24, and Part C baseline following administration of rhNaGlu.

FIG. 27 shows cognitive age equivalence (AEq) for individual subjects at baseline, week 24, and Part C baseline following administration of rhNaGlu.

FIG. 28 shows developmental quotient values (DQ) for individual subjects at baseline, week 24, and Part C baseline following administration of rhNaGlu.

FIG. 29A shows cognitive age equivalence (AEq) for individual subjects at baseline, week 24, and Part C baseline following administration of rhNaGlu, in comparison with preliminary data from MPSIIIB Natural History study (https://clinicaltrials.gov/ct2/show/study/NCT01509768). Colored square symbols are the individual subjects (N=11, Example 9), data at day 0 baseline, 6 months, Part C baseline; black solid symbols are from MPSIIIB Natural History study (N=13), data at baseline, 6 months, 12 months.

FIG. 29B shows developmental quotient values (DQ) for individual subjects at baseline, week 24, and Part C baseline following administration of rhNaGlu, in comparison with preliminary data from MPSIIIB Natural History study (https://clinicaltrials.gov/ct2/show/study/NCT01509768). Colored square symbols are the individual subjects (N=11, Example 9); black solid symbols are from MPSIIIB Natural History study (N=13).

FIG. 30A shows cognitive age equivalence (AEq) for individual subjects at baseline, week 24, and Part C baseline following administration of rhNaGlu, in comparison with MPSIIIA Natural History data. Colored symbols are the individual subjects (N=11, Example 9), data at day 0 baseline, week 24, and Part C baseline; solid black dots are from MPSIIIA Natural History; a normative growth line extends from the origin. See Shapiro et al., J. Ped. 2016, 170:278-287, FIG. 1A; also shown in FIG. 1A of Shapiro et al. is the growth trajectory for the rapidly progressing (RP) group.

FIG. 30B shows developmental quotient values (DQ) for individual subjects at baseline, week 24, and Part C baseline following administration of rhNaGlu, in comparison with MPSIIIA Natural History data. Colored symbols are the individual subjects (N=11, Example 9); solid black dots are from MPSIIIA Natural History (Shapiro et al., J. Ped. 2016, 170:278-287, FIG. 1B).

FIG. 31A shows age equivalence (AEq) plotted against chronological age for individual subjects in two natural history studies: MPSIIIA and MPSIIIB. Data is shown for MPSIIIB Rapid Progressors (early onset, N=14) (solid green circles); MPSIIIA Rapid Progressors (early onset, N=19) (solid red circles); MPSIIIB Slow Progressors (late onset, N=4) (open green circles), and MPSIIIA Slow Progressors (late onset, N=6) (open red circles).

FIG. 31B shows cognitive developmental quotient (DQ) plotted against chronological age for individual subjects in two natural history studies: MPSIIIA and MPSIIIB. Data is shown for MPSIIIB Rapid Progressors (early onset, N=14) (solid green circles); MPSIIIA Rapid Progressors (early onset, N=19) (solid red circles); MPSIIIB Slow Progressors (late onset, N=4) (open green circles), and MPSIIIA Slow Progressors (late onset, N=6) (open red circles).

FIG. 32 shows developmental age plotted against chronological age for individual subjects in an MPSIIIA natural history study, using an alternative assessment tool, Mullen Scales of Early Learning, to calculate developmental age.

FIG. 33 shows Vineland age equivalence (AEq) for individual subjects at baseline, week 24, and Part C baseline following administration of rhNaGlu.

FIG. 34 shows Vineland developmental quotient (DQ) for individual subjects at baseline, week 24, and Part C baseline following administration of rhNaGlu.

### DETAILED DESCRIPTION

### Definitions

Some of the definitions and abbreviations used herein include the following: aa, amino acid(s); bp, base pair(s); CDS, coding sequence cDNA, DNA complementary to an RNA; GalNac, N-acetylgalactosamine; Gal, galactose; GlcNac, N-acetylglucosamine; nt, nucleotide(s); kb, 1,000 base pairs; μg, microgram; mL, milliliter; ng, nanogram; and nt, nucleotide.

Certain definitions are set forth herein to illustrate and define the meaning and scope of the various terms used to describe the invention herein.

The term “avian” as used herein refers to any species, subspecies or strain of organism of the taxonomic class ava, such as, but not limited to, chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Ausstralorp, Minorca, Amrox, California Gray, Italian Partridge-colored), as well as strains of turkeys, pheasants, quails, duck, ostriches and other poultry commonly bred in commercial quantities.

The phrases “based on” and “derived from” typically mean obtained from, in whole or in part. For example, a retroviral vector being based on or derived from a particular retrovirus or based on a nucleotide sequence of a particular retrovirus mean that the genome of the retroviral vector contains a substantial portion of the nucleotide sequence of the genome of the particular retrovirus. The substantial portion can be a particular gene or nucleotide sequence such as the nucleotide sequence encoding the gag, pol and/or env proteins or other structural or functional nucleotide sequence of the virus genome such as sequences encoding the long terminal repeats (LTRs) or can be substantially the complete retrovirus genome, for example, most (e.g., more than 60% or more than 70% or more than 80% or more than 90%) or all of the retrovirus genome, as will be apparent from the context in the specification as the knowledge of one skilled in the art. Examples of retroviral vectors that are based on or derived from a retrovirus are the NL retroviral vectors (e.g., NLB) which are derived from the avian leukosis retrovirus (“ALV”) as disclosed in Cosset et al., Journal of Virology (1991) vol. 65, p 3388-3394.

The term “coding sequence” and “coding region” as used herein refer to nucleotide sequences and nucleic acid sequences, including both RNA and DNA, that encode genetic information for the synthesis of an RNA, a protein, or any portion of an RNA or protein.

Nucleotide sequences that are not naturally part of a particular organism's genome or are introduced at a non-native site in the organism's genome are referred to as “foreign” nucleotide sequences, “heterologous” nucleotide sequences, “recombinant” nucleotide sequences or “exogenous” nucleotide sequences. In addition, a nucleotide sequence that has been isolated and then reintroduced into the same type (e.g., same species) of organism is not considered to be a naturally occurring part of a particular organism's genome and is therefore considered exogenous or heterologous. “Heterologous proteins” or “exogenous proteins” can be proteins encoded by foreign, heterologous or exogenous nucleotide sequences and therefore are often not naturally expressed in a cell of the host organism.

As used herein, the terms “exogenous,” “heterologous” and “foreign” with reference to nucleic acids, such as DNA and RNA, are used interchangeably and refer to nucleic acid that does not occur naturally as part of a chromosome, a genome or cell in which it is present or which is found in a location(s) and/or in amounts that differ from the location(s) and/or amounts in which it occurs in nature. It can be nucleic acid that is not endogenous to the genome, chromosome or cell and has been exogenously introduced into the genome, chromosome or cell. Examples of heterologous DNA include, but are not limited to, DNA that encodes a gene product or gene product(s) of interest, for example, for production of an encoded protein. Examples of heterologous DNA include, but are not limited to, DNA that encodes traceable marker proteins, DNA that encodes therapeutic proteins. The terms “heterologous” and “exogenous” can refer to a biomolecule such as a nucleic acid or a protein which is not normally found in a certain cell, tissue or substance produced by an organism or is not normally found in a certain cell, tissue or substance produced by an organism in an amount or location the same as that found to occur naturally. For example, a protein that is heterologous or exogenous to an egg is a protein that is not normally found in the egg.

The term “construct” as used herein refers to a linear or circular nucleotide sequence such as DNA that has been assembled from more than one segments of nucleotide sequence which have been isolated from a natural source or have been chemically synthesized, or combinations thereof.

The term “complementary” as used herein refers to two nucleic acid molecules that can form specific interactions with one another. In the specific interactions, an adenine base within one strand of a nucleic acid can form two hydrogen bonds with thymine within a second nucleic acid strand when the two nucleic acid strands are in opposing polarities. Also in the specific interactions, a guanine base within one strand of a nucleic acid can form three hydrogen bonds with cytosine within a second nucleic acid strand when the two nucleic acid strands are in opposing polarities. Complementary nucleic acids as referred to herein, can further comprise modified bases wherein a modified adenine may form hydrogen bonds with a thymine or modified thymine, and a modified cytosine may form hydrogen bonds with a guanine or a modified guanine.

The term “expressed” or “expression” as used herein refers to the transcription of a coding sequence to yield an RNA molecule at least complementary in part to a region of one of the two nucleic acid strands of the coding sequence. The term “expressed” or “expression” as used herein can also refer to the translation of an mRNA to produce a protein or peptide.

The term “expression vector” as used herein refers to a nucleic acid vector that comprises a gene expression controlling region, such as a promoter or promoter component, operably linked to a nucleotide sequence encoding at least one polypeptide.

The term “fragment” as used herein can refer to, for example, an at least about 10, 20, 50, 75, 100, 150, 200, 250, 300, 500, 1000, 2000, 5000, 6,000, 8,000, 10,000, 20,000, 30,000, 40,000, 50,000 or 60,000 nucleotide long portion of a nucleic acid that has been constructed artificially (e.g., by chemical synthesis) or by cleaving a natural product into multiple pieces, using restriction endonucleases or mechanical shearing, or enzymatically, for example, by PCR or any other polymerizing technique known in the art, or expressed in a host cell by recombinant nucleic acid technology known to one of skill in the art. The term “fragment” as used herein can also refer to, for example, an at least about 5, 10, 15, 20, 25, 30, 40, or 50 amino acid residues less than a full length amino acid sequence for NaGlu (i.e., amino acid sequence 24-743 of SEQ ID NO:1), which portion is cleaved from a naturally occurring amino acid sequence by proteolytic cleavage by at least one protease, or is a portion of the naturally occurring amino acid sequence synthesized by chemical methods or using recombinant DNA technology (e.g., expressed from a portion of the nucleotide sequence encoding the naturally occurring amino acid sequence) known to one of skill in the art. “Fragment” may also refer to a portion, for example, of about 50%, about 60%, about 70%, about 80%, about 90%, about 95% or about 99% of a particular nucleotide sequence or amino acid sequence.

“Functional portion” and “functional fragment” can be used interchangeably and as used herein mean a portion or fragment of a whole capable of performing, in whole or in part, a function of the whole. For example, a biologically functional portion of a molecule means a portion of the molecule that performs a biological function of the whole or intact molecule. Functional portions may be of any useful size. For example, a functional fragment may range in size from about 20 bases in length to a length equal to the entire length of the specified sequence minus one nucleotide. In another example, a functional fragment may range in size from about 50 bases in length to a length equal to the entire length of the specified sequence minus one nucleotide. In another example, a functional fragment may range in size from about 50 bases in length to about 20 kb in length. In another example, a functional fragment may range in size from about 500 bases in length to about 20 kb in length. In another example, a functional fragment may range in size from about 1 kb in length to about 20 kb in length. In another example, a functional fragment may range in size from about 0.1 kb in length to about 10 kb in length. In another example, a functional fragment may range in size from about 20 bases kb in length to about 10 kb in length.

The term “fully transgenic” or “germline transgenic” refers to an animal such as an avian that contains at least one copy of a transgene in essentially all of its cells.

The term “gene expression controlling region” as used herein refers to nucleotide sequences that are associated with a coding sequence and which regulate, in whole or in part, expression of the coding sequence, for example, regulate, in whole or in part, the transcription of the coding sequence. Gene expression controlling regions may be isolated from a naturally occurring source or may be chemically synthesized and can be incorporated into a nucleic acid vector to enable regulated transcription in appropriate cells. The “gene expression controlling regions” may precede, but is not limited to preceding, the region of a nucleic acid sequence that is in the region 5′ of the end of a coding sequence that may be transcribed into mRNA.

As used herein, the terms “glycan,” “glycan structure,” “glycan moiety,” “oligosaccharide,” “oligosaccharide structure,” “glycosylation pattern,” “glycosylation profile,” and “glycosylation structure” have essentially the same meaning and each refers to one or more structures which are formed from sugar residues and are attached to glycosylated protein such as human NaGlu. For example, “N-glycan” or “N-linked glycan” refers to a glycan structure attached to a nitrogen of asparagine or arginine side-chain of the glycosylated protein. “O-glycan” or “O-linked glycan” refers to a glycan structure attached to the hydroxyl oxygen of serine, threonine, tyrosine, hydroxylysine, or hydroxyproline side chain of the glycosylate protein.

As used herein, “host cells” refers to cells that harbor vectors constructed using recombinant DNA techniques and encoding at least one heterologous gene.

The term “isolated nucleic acid” as used herein covers, for example, (a) a DNA which has the sequence of part of a naturally occurring genomic molecule but is not flanked by at least one of the sequences that flank that part of the molecule in the genome of the species in which it naturally occurs; (b) a nucleic acid which has been incorporated into a vector or into the genomic DNA of a prokaryote or eukaryote in a manner such that the resulting vector or genomic DNA is not identical to naturally occurring DNA from which the nucleic acid was obtained; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, i.e., a gene encoding a fusion protein, and (e) a recombinant nucleotide sequence that is part of a hybrid sequence that is not naturally occurring. Isolated nucleic acid molecules of the present invention can include, for example, natural allelic variants as well as nucleic acid molecules modified by nucleotide deletions, insertions, inversions, or substitutions.

The term “nucleic acid” as used herein refers to any linear or sequential array of nucleotides and nucleosides, for example cDNA, genomic DNA, mRNA, tRNA, oligonucleotides, oligonucleosides and derivatives thereof. For ease of discussion, non-naturally occurring nucleic acids may be referred to herein as constructs. Nucleic acids can include bacterial plasmid vectors including expression, cloning, cosmid and transformation vectors such as, animal viral vectors such as, but not limited to, modified adenovirus, influenza virus, polio virus, pox virus, retroviruses such as avian leukosis virus (ALV) retroviral vector, a murine leukemia virus (MLV) retroviral vector, and a lentivirus vector, and the like and fragments thereof. In addition, the nucleic acid can be an LTR of an avian leukosis virus (ALV) retroviral vector, a murine leukemia virus (MLV) retroviral vector, or a lentivirus vector and fragments thereof. Nucleic acids can also include NL vectors such as NLB, NLD and NLA and fragments thereof and synthetic oligonucleotides such as chemically synthesized DNA or RNA. Nucleic acids can include modified or derivatized nucleotides and nucleosides such as, but not limited to, halogenated nucleotides such as, but not only, 5-bromouracil, and derivatized nucleotides such as biotin-labeled nucleotides.

The term “vector” and “nucleic acid vector” as used herein refers to a natural or synthetic single or double stranded plasmid or viral nucleic acid molecule that can be transfected or transformed into cells and replicate independently of, or within, the host cell genome. A circular double stranded vector can be linearized by treatment with an appropriate restriction enzyme based on the nucleotide sequence of the vector. A nucleic acid can be inserted into a vector by cutting the vector with restriction enzymes and ligating the desired pieces together, as is understood in the art. A typical vector can be comprised of the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements can be omitted in specific applications. The nucleic acid cassette can include a restriction site for insertion of the nucleic acid sequence to be expressed. In a functional vector the nucleic acid cassette contains the nucleic acid sequence to be expressed including translation initiation and termination sites. An intron optionally can be included in the construct, for example, 5′ to the coding sequence. A vector is constructed so that the particular coding sequence is located in the vector with the appropriate regulatory sequences, the positioning and orientation of the coding sequence with respect to the control sequences being such that the coding sequence is transcribed under the “control” of the control or regulatory sequences. Modification of the sequences encoding the particular protein of interest can be desirable to achieve this end. For example, in some cases it can be necessary to modify the sequence so that it can be attached to the control sequences with the appropriate orientation, or to maintain the reading frame. The control sequences and other regulatory sequences can be ligated to the coding sequence prior to insertion into a vector. Alternatively, the coding sequence can be cloned directly into an expression vector which already contains the control sequences and an appropriate restriction site which is in reading frame with and under regulatory control of the control sequences.

The term “operably linked” refers to an arrangement of elements wherein the components so described are configured so as to perform their usual function. Gene expression controlling regions or promoter(s) (e.g., promoter components) operably linked to a coding sequence are capable of effecting the expression of the coding sequence. The controlling sequence(s) or promoter need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

“Overexpression”, as used herein, refers to the production of a gene product in transgenic organisms that exceeds levels of production in normal or non-transformed organisms.

The term “oviduct” or “oviduct tissue” refers to a tissue of an avian oviduct, such as the magnum, e.g., tubular gland cells, where proteins are produced with N-linked oligosaccharides that contain increased amounts of mannose and mammose-6-phosphate (M6P) and substantially reduced amounts of galactose and/or sialic acid relative to that of proteins produced in other tissue of the avian such as liver or kidney tissue.

The term “oviduct-specific promoter” as used herein refers to promoters and promoter components which are functional, i.e., provide for transcription of a coding sequence, to a large extent, for example, primarily (i.e., more than 50% of the transcription product produced in the animal by a particular promoter type being produced in oviduct cells) or exclusively in oviduct cells of a bird. Examples of oviduct specific promoters include, but are not limited to, ovalbumin promoter, ovomucoid promoter, ovoinhibitor promoter, lysozyme promoter and ovotransferrin promoter and functional portions of these promoters, e.g., promoter components. By limiting the expression of NaGlu protein to the magnum using oviduct specific promoters, deleterious physiological effects to the bird as result of expression of these enzymes in other tissues of the bird can be minimized.

The terms “percent sequence identity,” “percent identity,” “% identity,” “percent sequence homology,” “percent homology,” “% homology” and “percent sequence similarity” can each refer to the degree of sequence matching between two nucleic acid sequences or two amino acid sequences. Such sequence matching can be determined using the algorithm of Karlin & Altschul (1990) Proc. Natl. Acad. Sci. 87: 2264-2268, modified as in Karlin & Altschul (1993) Proc. Natl. Acad. Sci. 90: 5873-5877. Such an algorithm is incorporated into the NBLAST and) XBLAST programs of Altschul et al. (1990) T. Mol. Biol. Q15: 403-410. BLAST nucleotide searches are performed with the NBLAST program, score=100, wordlength=12, to obtain nucleotide sequences homologous to a disclosed nucleic acid molecule. BLAST protein searches are performed with the XBLAST program, score=50, wordlength=3, to obtain amino acid sequences homologous to a reference amino acid sequence. To obtain gapped alignments for comparison purposes, Gapped BLAST is utilized as described in Altschul et al. (1997) Nucl. Acids Res. 25: 3389-3402. When utilizing BLAST and Gapped BLAST programs, the default parameters of the respective programs (e.g., XBLAST and NBLAST) are used. Other algorithms, programs and default settings may also be suitable such as, but not only, the GCG-Sequence Analysis Package of the U.K. Human Genome Mapping Project Resource Centre that includes programs for nucleotide or amino acid sequence comparisons. A sequence may be at least 50%, 60%, 70%, 75%, 80%, 85%, 90%, 91%, 92%, 93%, 94%, 95%, 96%, 97%, 98%, 99% or more identical to another sequence, e.g., the NaGlu protein sequence identified herein.

The term “avian derived” refers to a composition or substance produced by or obtained from a bird, poultry or avian. “Avian” refers to birds that can be kept as livestock, including but not limited to, chickens, duck, turkey, quail and ratites. For example, “avian derived” can refer to chicken derived, turkey derived and/or quail derived.

The terms “polynucleotide,” “oligonucleotide”, “nucleotide sequence” and “nucleic acid sequence” can be used interchangeably herein and include, but are not limited to, coding sequences, i.e., polynucleotide(s) or nucleic acid sequence(s) which are transcribed and translated into polypeptide in vitro or in vivo when placed under the control of appropriate regulatory or control sequences; controlling sequences, e.g., translational start and stop codons, promoter sequences, ribosome binding sites, polyadenylation signals, transcription factor binding sites, transcription termination sequences, upstream and downstream regulatory domains, enhancers, silencers, DNA sequences to which a transcription factor(s) binds and alters the activity of a gene's promoter either positively (induction) or negatively (repression) and the like. No limitations as to length or to synthetic origin are suggested by the terms described herein.

As used herein the terms “polypeptide” and “protein” refer to a polymer of amino acids, for example, three or more amino acids, in a serial array, linked through peptide bonds. The term “polypeptide” includes proteins, protein fragments, protein analogues, oligopeptides and the like. The term “polypeptides” includes polypeptides as defined above that are encoded by nucleic acids, produced through recombinant technology (e.g., isolated from a transgenic bird), or synthesized. The term “polypeptides’ further contemplates polypeptides as defined above that include chemically modified amino acids or amino acids covalently or noncovalently linked to labeling ligands.

The term “promoter” as used herein refers to a DNA sequence useful to initiate transcription by an RNA polymerase in an avian cell. A “promoter component” is a DNA sequence that can, by itself or in combination with other DNA sequences, effect or facilitate transcription. Promoter components can be functional fragments of promoters.

The terms “recombinant nucleic acid” and “recombinant DNA” as used herein refer to combinations of at least two nucleic acid sequences that are not naturally found in a eukaryotic or prokaryotic cell. The nucleic acid sequences may include, but are not limited to, nucleic acid vectors, gene expression regulatory elements, origins of replication, suitable gene sequences that when expressed confer antibiotic resistance, protein-encoding sequences and the like. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

As used herein, the term “regulatory” sequences or elements include promoters, enhancers, terminators, stop codons, and other elements that can control gene expression.

A “retrovirus”, “retroviral particle,” “transducing particle,” or “transduction particle” refers to a replication-defective or replication-competent virus capable of transducing non-viral DNA or RNA into a cell.

A “SIN vector” refers to a self-inactivating vector. In particular, a SIN vector is a retroviral vector having an altered genome such that upon integration into genomic DNA of the target cell (e.g., avian embryo cells), the 5′ LTR of the integrated retroviral vector will not function as a promoter. For example, a portion or all of the nucleotide sequence of the retroviral vector that results in the U3 region of the 5′ LTR of the retroviral vector once integrated can be deleted or altered in order to reduce or eliminate promoter activity of the 5′ LTR. In certain examples, deletion of the CAAT box and/or the TAATA box from U3 of the 5′ LTR can result in a SIN vector, as is understood in the art.

The term “sense strand” as used herein refers to a single stranded DNA molecule from a genomic DNA that can be transcribed into RNA and translated into the natural polypeptide product of the gene. The term “antisense strand” as used herein refers to the single strand DNA molecule of a genomic DNA that is complementary with the sense strand of the gene.

A “therapeutic protein” or “pharmaceutical protein” is a substance that, in whole or in part, makes up a drug. In particular, “therapeutic proteins” and “pharmaceutical proteins” include an amino acid sequence which in whole or in part makes up a drug.

The terms “promoter,” “transcription regulatory sequence” and “promoter component” as used herein refer to nucleotide which regulates the transcriptional expression of a coding sequence. Exemplary transcription regulatory sequences include enhancer elements, hormone response elements, steroid response elements, negative regulatory elements, and the like. The “transcription regulatory sequence” can be isolated and incorporated into a vector to enable regulated transcription in appropriate cells of portions of the vector DNA. The “transcription regulatory sequence” can precede, but is not limited to, the region of a nucleic acid sequence that is in the region 5′ of the end of a protein coding sequence that is transcribed into mRNA. Transcriptional regulatory sequence can also be located within a protein coding region, for example, in regions of a gene that are identified as “intron” regions.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with certain concentrations of salt, for example, but without limitation, a calcium or magnesium salt, or exposing the cells to an electric field, detergent, or liposome material, to render the host cell competent for the uptake of the nucleic acid molecules.

As used herein, a “transgenic animal” is any non-human animal, such as an avian species, including the chicken, in which one or more of the cells of the animal contain heterologous nucleic acid introduced by way of human intervention, such as by transgenic techniques known in the art (see, for example, U.S. patent publication No. 2007/0243165, published Oct. 18, 2007, the disclosure of which is incorporated in its entirety herein by reference) including those disclosed herein. The nucleic acid is introduced into an animal, directly or indirectly by introduction into a cell (e.g., egg or embryo cell) by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule can be integrated within a chromosome, or it may be extrachromosomally replicating DNA. In the typical transgenic animal, the transgene can cause cells to express a recombinant form of the target protein or polypeptide. The terms “chimeric animal” or “mosaic animal” are used herein to refer to animals in which a transgene is found, or in which the recombinant nucleotide sequence is expressed, in some but not all cells of the animal. A germ-line chimeric animal contains a transgene in its germ cells and can give rise to an offspring transgenic animal in which most or all cells of the offspring will contain the transgene.

As used herein, the term “transgene” means a nucleic acid sequence (encoding, for example, a human NaGlu protein) that is partly or entirely heterologous, i.e., foreign, to the animal or cell into which it is introduced, or, is partly or entirely homologous to an endogenous gene of the transgenic animal or cell into which it is introduced, but which is designed to be inserted, or is inserted, into the animal or cell genome in such a way as to alter the genome of the organism into which it is inserted (e.g., it is inserted at a location which differs from that of the natural gene or its insertion results in a knockout).

As used herein, the term “enzyme replacement therapy (ERT)” refers to a therapeutic strategy for correcting an enzyme deficiency in a subject by administering the missing enzyme to a subject. For lysosomal enzyme replacement therapy to be effective, the therapeutic enzyme must be delivered to lysosomes in the appropriate cells in tissues where the storage defect is manifested. In one embodiment, the enzyme may be administered to the subject intravenously, intrathecally, intracerebrally, intraventricularly, or intraparenchymaly. In one embodiment, the enzyme is able to cross the blood brain barrier (BBB). Without intending to be limited by mechanism, it is believed that as the blood perfuses patient tissues, enzyme is taken up by cells and transported to the lysosome, where the enzyme acts to eliminate material that has accumulated in the lysosomes due to the enzyme deficiency.

The terms “treat,” “treating,” and “treatment” refer to methods of alleviating, abating, or ameliorating a disease or symptom, preventing an additional symptom, ameliorating or preventing an underlying cause of a symptom, inhibiting a disease or condition, arresting the development of a disease or condition, relieving a disease or condition, causing regression of a disease or condition, relieving a condition caused by the disease or condition, or stopping a symptom of the disease or condition either prophylactically and/or after the symptom has occurred.

“Therapeutically effective dose” as used herein refers to the dose (e.g., amount and/or interval) of drug required to produce an intended therapeutic response (e.g., reduction of heparan sulfate levels and/or increase in NaGlu activity in a target tissue). A therapeutically effective dose refers to a dose that, as compared to a corresponding subject who has not received such a dose, results in improved treatment, healing, prevention, or amelioration of a disease, disorder, or side effect, or a decrease in the rate of the occurrence or advancement of a disease or disorder. The term also includes within its scope, doses effective to enhance physiological functions.

As used herein, the term “subject” or “patient” is intended to include human and non-human animals. Non-human animals include all vertebrates, e.g., mammals and non-mammals, such as non-human primates, sheep, dogs, cats, cows, horses, chickens, amphibians, and reptiles. Preferred subjects include human subjects having a NaGlu deficiency or NaGlu associated disease.

As used herein a “NaGlu associated disease” is a disease or condition which is mediated by NaGlu activity or is associated with aberrant NaGlu expression or activity. An example of an NaGlu associated disease includes, but is not limited to, NaGlu deficiency such as Sanflippo Syndrome B (also known as mucopolysaccharidosis type IIIB).

“Intravenous injection,” often medically referred to as IV push or bolus injection, refers to a route of administration in which a syringe is connected to the IV access device and the medication is injected directly, typically rapidly and occasionally up to a period of 15 minutes if it might cause irritation of the vein or a too-rapid effect. Once a medicine has been injected into the fluid stream of the IV tubing, there must be some means of ensuring that it gets from the tubing to the patient. Usually this is accomplished by allowing the fluid stream to flow normally and thereby carry the medicine into the bloodstream. However, in some cases a second fluid injection, sometimes called a “flush,” is used following the first injection to facilitate the entering of the medicine into the bloodstream.

“Intravenous infusion” refers to a route of administration in which medication is delivered over an extended period of time. For example, the medication can be delivered to a patient over a period of time between 1 and 8 hours. The medication can also be delivered to a patient over a period of about 1, about 2, about 3, about 4, about 5, about 6, about 7, or about 8 hours. To accomplish an intravenous infusion, an IV gravity drip or an IV pump can be used.

IV infusion is typically used when a patient requires medications only at certain times and does not require additional intravenous fluids (e.g., water solutions which can contain sodium, chloride, glucose, or any combination thereof) such as those that restore electrolytes, blood sugar, and water loss.

### Abbreviations

BBB blood brain barrier
cDNA complementary DNA
CNS central nervous system
CSF cerebrospinal fluid
Fuc Fucose
GAG glycosaminoglycan
Gal Galactose
GalNAc N-Acetylgalactosamine
Glc Glucose
GlcNAc N-Acetylglucosamine
HGF hepatocyte growth factor
HS heparan sulfate
HSD heparan sulfate disaccharides
IV intravenous
IT intrathecal
Man mannose
M6P mannose-6-phosphate
MPSIIIB Mucopolysaccharidosis IIIB (Sanfilippo B Syndrome)
rhNaGlu recombinant human alpha-N-acetyl-glucosaminidase
NeuNAc N-acetylneuraminic acid

### Enzyme Replacement Therapy

This disclosure relates to compositions comprising recombinant human NaGlu protein (rhNaGlu) useful for therapy, for example, in the treatment of NaGlu associated diseases, e.g., Sanfilippo Syndrome B and to particular therapeutic methods.

The recombinant human NaGlu protein described herein contains sufficient amount of oligosaccharides (e.g., mannose and phosphorylated mannose (i.e., M6P)) to allow efficient cellular uptake via mannose and/or M6P receptor-mediated endocytosis and be correctly targeted into human cells, and has one or more glycosylation patterns that allow the rhNaGlu to efficiently cross the blood brain barrier (BBB) when administered intravenously. See, e.g., patent publications WO2013/055888; US2013/0095092; US2014/0255383; and WO2016/054025.

Direct administration of mammalian cell-produced rhNaGlu having the native amino acid sequence into the central nervous system (CNS) (e.g., intrathecal administration into the cerebrospinal fluid (CSF)) of NaGlu deficient mice has been attempted, but failed to demonstrate successful biodistribution of the enzyme to the brain. In the present disclosure, however, it is shown that intravenous infusion of rhNaGlu is able to reduce total heparan sulfate in the CNS, and produce positive neurocognitive effects, indicating that intravenously infused rhNaGlu has successfully crossed the BBB.

Because MPS IIIB deficiency is a genetic disorder that will likely require life-long enzyme replacement therapy, the ability to treat MPS IIIB patients in a systemic manner (e.g., intravenous administration) on a long-term basis achieves a significant advancement in patient care as it drastically enhances the quality of life for not only patients, but also care providers.

### I. Methods of Treatment

The present invention provides methods of treating NaGlu-associated diseases, e.g., Sanfilippo Syndrome B.

In particular, this disclosure relates to methods for treating Sanfilippo Syndrome B by enzyme replacement therapy. In some embodiments, the methods involve the intravenous administration of an effective amount of recombinant human NaGlu to a subject in need thereof every other week (QOW); however, other treatment schedules, either more frequent or less frequent, are also encompassed by the treatment methods as described herein. In some aspects, the method includes administering a dose of about 0.3 mg/kg to about 10 mg/kg of recombinant human NaGlu every other week. For example, a dose of about 0.3 mg/kg, about 1 mg/kg, about 3 mg/kg, about 5 mg/kg, about 6 mg/kg, about 7 mg/kg, about 8 mg/kg, about 9 mg/kg, or about 10 mg/kg or higher can be administered every other week. Other dosage amounts are also encompassed by the treatment methods as described herein. Preferably, the intravenous administration is by intravenous infusion as described herein, such as a two hour intravenous infusion.

As described and exemplified herein, intravenous administration of human NaGlu in accordance with the methods resulted in a decrease in total heparan sulfate levels in the cerebral spinal fluid in MPSIIIB patients in comparison to base line levels before administration. In addition, intravenous administration of human NaGlu in accordance with the methods also stabilized or improved brain morphomety (e.g., cortical grey matter volume) and neurocognitive and behavioral scores (i.e., Bayley Scales of Infant Development Third Edition (BSID-III), Kaufman Assessment Battery for Children Second Edition (KABC-II), and Vineland Adaptive Behavior Scales—Second Edition (VABS-II)). Accordingly, in some aspects the effective amount of recombinant human NaGlu that is intravenously administered is sufficient to 1) decrease total heparan sulfate levels in the cerebral spinal fluid in MPSIIIB patients in comparison to base line levels before administration; 2) stabilized or improved brain morphomety (e.g., cortical gray matter volume) in comparison to base line levels before administration; and/or 3) improve neurocognition and/or behavioral adaptation, as measured by various indicators or scores (e.g., Bayley Scales of Infant Development Third Edition (BSID-III) and Kaufman Assessment Battery for Children Second Edition (KABC-II), or Vineland Adaptive Behavior Scales—Second Edition (VABS-II)) in comparison to base line levels before administration. The treatment methods encompass the use of any subjective or objective measure to determine changes in heparan sulfate levels, stabilized or improved brain morphometry, and improved neurocognition or behavioral adaptation. For example, improved neurocognitive performance can be measured by scores on standardized tests or assessments, or by measures used to assess alleviation, amelioration, relief, inhibition, delay of onset, reduction in severity and/or incidence of neurological impairment in an MPSIIIB patient.

Recombinant NaGlu employed in accordance with the invention includes recombinant NaGlu which can be produced in any useful protein expression system including, without limitation, cell culture (e.g., CHO cells, COS cells), bacteria such as E. coli, transgenic animals such as mammals and avians (e.g., chickens, duck, and turkey) and in plant systems (e.g., duck weed and tobacco plants). In one embodiment, the recombinant NaGlu is produced in a transgenic animal, such as an avian. In one embodiment, the method comprises administering to the subject a recombinant human NaGlu protein (rhNaGlu), for instance a recombinant human NaGlu protein containing a sufficient amount of oligosaccharides (e.g., mannose and phosphorylated mannose (i.e., M6P)), in an amount sufficient to treat (e.g., reduce, ameliorate) or prevent one or more symptoms of a NaGlu deficiency or NaGlu associated disease. The recombinant human NaGlu protein can be administered therapeutically or prophylactically, or both. The recombinant human NaGlu protein (rhNaGlu) can be administered to the subject, alone or in combination with other therapeutic modalities as described herein.

The therapeutic methods of the present invention encompass any route of administration which facilitates the uptake or transport of the recombinant human NaGlu protein into the pertinent organs and tissues. In one embodiment, the methods of the invention include delivering the recombinant human NaGlu proteins of the invention to the CNS (central nervous system), the kidney, or the liver of a subject for the treatment of a NaGlu associated disease (e.g., NaGlu deficiency). For example, the recombinant human NaGlu protein may be administered to the patient intravenously (e.g., via intravenous injection or intravenous infusion) and surprisingly crosses the blood brain barrier (BBB) of the subject having NaGlu deficiency. In another embodiment, the recombinant human NaGlu protein is administered to the patient intrathecally.

A. Device for Intrathecal Delivery

Various devices may be used for intrathecal delivery according to the present invention. In some embodiments, a device for intrathecal administration contains a fluid access port (e.g., injectable port); a hollow body (e.g., catheter) having a first flow orifice in fluid communication with the fluid access port and a second flow orifice configured for insertion into spinal cord; and a securing mechanism for securing the insertion of the hollow body in the spinal cord. As a non-limiting example, a suitable securing mechanism contains one or more nobs mounted on the surface of the hollow body and a sutured ring adjustable over the one or more nobs to prevent the hollow body (e.g., catheter) from slipping out of the spinal cord. In various embodiments, the fluid access port comprises a reservoir. In some embodiments, the fluid access port comprises a mechanical pump (e.g., an infusion pump). In some embodiments, an implanted catheter is connected to either a reservoir (e.g., for bolus delivery), or an infusion pump. The fluid access port may be implanted or external.

In some embodiments, intrathecal administration may be performed by either lumbar puncture (i.e., slow bolus) or via a port-catheter delivery system (i.e., infusion or bolus). In some embodiments, the catheter is inserted between the laminae of the lumbar vertebrae and the tip is threaded up the thecal space to the desired level (generally L3-L4).

Relative to intravenous administration, a single dose volume suitable for intrathecal administration is typically small. Typically, intrathecal delivery according to the present invention maintains the balance of the composition of the CSF as well as the intracranial pressure of the subject. In some embodiments, intrathecal delivery is performed absent the corresponding removal of CSF from a subject. In some embodiments, a suitable single dose volume may be e.g., less than about 10 mL, 8 mL, 6 mL, 5 mL, 4 mL, 3 mL, 2 mL, 1.5 mL, 1 mL, or 0.5 mL. In some embodiments, a suitable single dose volume may be about 0.5-5 mL, 0.5-4 mL, 0.5-3 mL, 0.5-2 mL, 0.5-1 mL, 1-3 mL, 1-5 mL, 1.5-3 mL, 1-4 mL, or 0.5-1.5 mL. In some embodiments, intrathecal delivery according to the present invention involves a step of removing a desired amount of CSF first. In some embodiments, less than about 10 mL (e.g., less than about 9 mL, 8 mL, 7 mL, 6 mL, 5 mL, 4 mL, 3 mL, 2 mL, 1 mL) of CSF is first removed before intrathecal administration. In those cases, a suitable single dose volume may be e.g., more than about 3 mL, 4 mL, 5 mL, 6 mL, 7 mL, 8 mL, 9 mL, 10 mL, 15 mL, or 20 mL. Ranges and values intermediate to the above recited ranges and values are also contemplated to be part of the invention.

Various other devices may be used to effect intrathecal administration of a therapeutic composition. For example, formulations containing desired enzymes may be given using an Ommaya reservoir which is in common use for intrathecally administering drugs for meningeal carcinomatosis (Lancet 2: 983-84, 1963). More specifically, in this method, a ventricular tube is inserted through a hole formed in the anterior horn and is connected to an Ommaya reservoir installed under the scalp, and the reservoir is subcutaneously punctured to intrathecally deliver the particular enzyme being replaced, which is injected into the reservoir. Other devices for intrathecal administration of therapeutic compositions or formulations to an individual are described in U.S. Pat. No. 6,217,552, the entire contents of which, as they relate to these devices, are incorporated herein by reference. Alternatively, the drug may be intrathecally given, for example, by a single injection, or continuous infusion. It should be understood that the dosage treatment may be in the form of a single dose administration or multiple doses.

For injection, compositions can be formulated in liquid solutions. In addition, the NaGlu enzyme may be formulated in solid form and re-dissolved or suspended immediately prior to use. Lyophilized forms are also included. The injection can be, for example, in the form of a bolus injection or continuous infusion (e.g., using infusion pumps) of the NaGlu enzyme.

In one embodiment, the NaGlu enzyme is administered by lateral cerebro ventricular injection into the brain of a subject. The injection can be made, for example, through a burr hole made in the subject's skull. In another embodiment, the enzyme and/or other pharmaceutical formulation is administered through a surgically inserted shunt into the cerebral ventricle of a subject. For example, the injection can be made into the lateral ventricles, which are larger. In some embodiments, injection into the third and fourth smaller ventricles can also be made.

In yet another embodiment, the pharmaceutical compositions used in the present invention are administered by injection into the cisterna magna, or lumbar area of a subject.

In another embodiment of the method of the invention, the pharmaceutically acceptable formulation provides sustained delivery, e.g., “slow release” of the enzyme or other pharmaceutical composition used in the present invention, to a subject for at least one, two, three, four weeks or longer periods of time after the pharmaceutically acceptable formulation is administered to the subject.

As used herein, the term “sustained delivery” refers to continual delivery of a pharmaceutical formulation in vivo over a period of time following administration, preferably at least several days, a week or several weeks. Sustained delivery of the composition can be demonstrated by, for example, the continued therapeutic effect of the enzyme over time (e.g., sustained delivery of the enzyme can be demonstrated by continued reduced amount of storage granules in the subject). Alternatively, sustained delivery of the enzyme may be demonstrated by detecting the presence of the enzyme in vivo over time.

B. Intravenous Delivery

As discussed above, one of the surprising features of the present invention is that the recombinant human NaGlu proteins of the invention are able to effectively and extensively diffuse across the blood brain barrier (BBB) and brain surface and penetrate various layers or regions of the brain, including deep brain regions, when administered intravenously. Recombinant human NaGlu can be administered via intravenous infusion or intravenous injection. The methods of the present invention effectively deliver the rhNaGlu proteins to various tissues, neurons or cells of the central nervous system (CNS), which are hard to target by existing CNS delivery methods. Furthermore, the methods of the present invention deliver sufficient amounts of the recombinant human NaGlu proteins to the blood stream and various peripheral organs and tissues.

C. Target Tissues

In some embodiments, the rhNaGlu of the invention is delivered to the central nervous system (CNS) of a subject. In some embodiments, the rhNaGlu of the invention is delivered to one or more of target tissues of brain, spinal cord, and/or peripheral organs. As used herein, the term “target tissue” refers to any tissue that is affected by the NaGlu associated disease to be treated or any tissue in which the deficient NaGlu is normally expressed. In some embodiments, target tissues include those tissues in which there is a detectable or abnormally high amount of enzyme substrate, for example stored in the cellular lysosomes of the tissue, in patients suffering from or susceptible to the NaGlu associated disease. In some embodiments, target tissues include those tissues that display a disease-associated pathology, symptom, or feature. In some embodiments, target tissues include those tissues in which the deficient NaGlu is normally expressed at an elevated level. As used herein, a target tissue may be a brain target tissue, a spinal cord target tissue and/or a peripheral target tissue. Exemplary target tissues are described in detail below.

D. Brain Target Tissues

In general, the brain can be divided into different regions, layers and tissues. For example, meningeal tissue is a system of membranes which envelops the central nervous system, including the brain. The meninges contain three layers, including dura matter, arachnoid matter, and pia matter. In general, the primary function of the meninges and of the cerebrospinal fluid is to protect the central nervous system. In some embodiments, a therapeutic protein in accordance with the present invention is delivered to one or more layers of the meninges.

The brain has three primary subdivisions, including the cerebrum, cerebellum, and brain stem. The cerebral hemispheres, which are situated above most other brain structures and are covered with a cortical layer. Underneath the cerebrum lies the brainstem, which resembles a stalk on which the cerebrum is attached. At the rear of the brain, beneath the cerebrum and behind the brainstem, is the cerebellum.

The diencephalon, which is located near the midline of the brain and above the mesencephalon, contains the thalamus, metathalamus, hypothalamus, epithalamus, prethalamus, and pretectum. The mesencephalon, also called the midbrain, contains the tectum, tegumentum, ventricular mesocoelia, and cerebral peduncels, the red nucleus, and the cranial nerve III nucleus. The mesencephalon is associated with vision, hearing, motor control, sleep/wake, alertness, and temperature regulation.

Regions of tissues of the central nervous system, including the brain, can be characterized based on the depth of the tissues. For example, CNS (e.g., brain) tissues can be characterized as surface or shallow tissues, mid-depth tissues, and/or deep tissues.

According to the present invention, the rhNaGlu of the invention may be delivered to any appropriate brain target tissue(s) associated with a particular disease to be treated in a subject. In some embodiments, the rhNaGlu of the invention is delivered to surface or shallow brain target tissue. In some embodiments, the rhNaGlu of the invention is delivered to mid-depth brain target tissue. In some embodiments, the rhNaGlu of the invention is delivered to deep brain target tissue. In some embodiments, the rhNaGlu of the invention is delivered to a combination of surface or shallow brain target tissue, mid-depth brain target tissue, and/or deep brain target tissue. In some embodiments, the rhNaGlu of the invention is delivered to a deep brain tissue at least 4 mm, 5 mm, 6 mm, 7 mm, 8 mm, 9 mm, 10 mm or more below (or internal to) the external surface of the brain. Ranges and values intermediate to the above recited ranges and values are also contemplated to be part of the invention.

In some embodiments, the rhNaGlu of the invention is delivered to one or more surface or shallow tissues of cerebrum. In some embodiments, the targeted surface or shallow tissues of the cerebrum are located within 4 mm from the surface of the cerebrum. In some embodiments, the targeted surface or shallow tissues of the cerebrum are selected from pia mater tissues, cerebral cortical ribbon tissues, hippocampus, Virchow Robin space, blood vessels within the VR space, the hippocampus, portions of the hypothalamus on the inferior surface of the brain, the optic nerves and tracts, the olfactory bulb and projections, and combinations thereof.

In some embodiments, the rhNaGlu of the invention is delivered to one or more deep tissues of the cerebrum. In some embodiments, the targeted surface or shallow tissues of the cerebrum are located at least 4 mm (e.g., 5 mm, 6 mm, 7 mm, 8 mm, 9 mm, or 10 mm) below (or internal to) the surface of the cerebrum. In some embodiments, targeted deep tissues of the cerebrum include the cerebral cortical ribbon. In some embodiments, targeted deep tissues of the cerebrum include one or more of the diencephalon (e.g., the hypothalamus, thalamus, prethalamus or subthalamus), metencephalon, lentiform nuclei, the basal ganglia, caudate, putamen, amygdala, globus pallidus, and combinations thereof.

In some embodiments, the rhNaGlu of the invention is delivered to one or more tissues of the cerebellum. In certain embodiments, the targeted one or more tissues of the cerebellum are selected from the group consisting of tissues of the molecular layer, tissues of the Purkinje cell layer, tissues of the Granular cell layer, cerebellar peduncles, and combination thereof. In some embodiments, therapeutic agents (e.g., enzymes) are delivered to one or more deep tissues of the cerebellum including, but not limited to, tissues of the Purkinje cell layer, tissues of the Granular cell layer, deep cerebellar white matter tissue (e.g., deep relative to the Granular cell layer), and deep cerebellar nuclei tissue.

In some embodiments, the rhNaGlu of the invention is delivered to one or more tissues of the brainstem. In some embodiments, the targeted one or more tissues of the brainstem include brain stem white matter tissue and/or brain stem nuclei tissue.

In some embodiments, the rhNaGlu of the invention is delivered to various brain tissues including, but not limited to, gray matter, white matter, periventricular areas, pia-arachnoid, meninges, neocortex, cerebellum, deep tissues in cerebral cortex, molecular layer, caudate/putamen region, midbrain, deep regions of the pons or medulla, and combinations thereof.

In some embodiments, the rhNaGlu of the invention is delivered to various cells in the brain including, but not limited to, neurons, glial cells, perivascular cells and/or meningeal cells. In some embodiments, a therapeutic protein is delivered to oligodendrocytes of deep white matter.

E. Spinal Cord Target Tissue

In general, regions or tissues of the spinal cord can be characterized based on the depth of the tissues. For example, spinal cord tissues can be characterized as surface or shallow tissues, mid-depth tissues, and/or deep tissues.

In some embodiments, the rhNaGlu of the invention are delivered to one or more surface or shallow tissues of the spinal cord. In some embodiments, a targeted surface or shallow tissue of the spinal cord is located within 4 mm from the surface of the spinal cord. In some embodiments, a targeted surface or shallow tissue of the spinal cord contains pia matter and/or the tracts of white matter.

In some embodiments, the rhNaGlu of the invention are delivered to one or more deep tissues of the spinal cord. In some embodiments, a targeted deep tissue of the spinal cord is located internal to 4 mm from the surface of the spinal cord. In some embodiments, a targeted deep tissue of the spinal cord contains spinal cord gray matter and/or ependymal cells.

In some embodiments, replacement enzymes (e.g., a NaGlu fusion protein) are delivered to neurons of the spinal cord.

F. Peripheral Target Tissues

As used herein, peripheral organs or tissues refer to any organs or tissues that are not part of the central nervous system (CNS). Peripheral target tissues may include, but are not limited to, blood system, liver, kidney, heart, endothelium, bone marrow and bone marrow derived cells, spleen, lung, lymph node, bone, cartilage, ovary and testis. In some embodiments, the rhNaGlu of the invention is delivered to one or more of the peripheral target tissues.

G. Biodistribution and Bioavailability

In various embodiments, once delivered to the target tissue, the rhNaGlu of the invention is localized intracellularly. For example, the rhNaGlu of the invention may be localized to exons, axons, lysosomes, mitochondria or vacuoles of a target cell (e.g., neurons such as Purkinje cells). For example, in some embodiments the rhNaGlu of the invention demonstrates translocation dynamics such that the rhNaGlu moves within the perivascular space (e.g., by pulsation-assisted convective mechanisms). In addition, active axonal transport mechanisms relating to the association of the administered protein or enzyme with neurofilaments may also contribute to or otherwise facilitate the distribution of the rhNaGlu proteins of the invention into the deeper tissues of the central nervous system.

In some embodiments, the rhNaGlu of the invention delivered according to the present invention may achieve therapeutically or clinically effective levels or activities in various targets tissues described herein. As used herein, a therapeutically or clinically effective level or activity is a level or activity sufficient to confer a therapeutic effect in a target tissue. The therapeutic effect may be objective (i.e., measurable by some test or marker) or subjective (i.e., subject gives an indication of or feels an effect). For example, a therapeutically or clinically effective level or activity may be an enzymatic level or activity that is sufficient to ameliorate symptoms associated with the disease in the target tissue (e.g., GAG storage).

In some embodiments, the rhNaGlu of the invention delivered according to the present invention may achieve an enzymatic level or activity that is at least 5%, 10%, 20%, 30%, 40%, 50%, 60%, 70%, 80%, 90%, 91%, 92%, 93%, 94%, 95%, 96%, 97%, 98% or 99% of the normal level or activity of the corresponding NaGlu enzyme in the target tissue. In some embodiments, the rhNaGlu of the invention delivered according to the present invention may achieve an enzymatic level or activity that is increased by at least 1-fold, 2-fold, 3 -fold, 4-fold, 5-fold, 6-fold, 7-fold, 8-fold, 9-fold or 10-fold as compared to a control (e.g., endogenous levels or activities without the treatment). In some embodiments, the rhNaGlu delivered according to the present invention may achieve an increased enzymatic level or activity at least approximately 10 nmol/hr/mg, 20 nmol/hr/mg, 40 nmol/hr/mg, 50 nmol/hr/mg, 60 nmol/hr/mg, 70 nmol/hr/mg, 80 nmol/hr/mg, 90 nmol/hr/mg, 100 nmol/hr/mg, 150 nmol/hr/mg, 200 nmol/hr/mg, 250 nmol/hr/mg, 300 nmol/hr/mg, 350 nmol/hr/mg, 400 nmol/hr/mg, 450 nmol/hr/mg, 500 nmol/hr/mg, 550 nmol/hr/mg or 600 nmol/hr/mg in a target tissue. Ranges and values intermediate to the above recited ranges and values are also contemplated to be part of the invention.

In some embodiments, inventive methods according to the present invention are particularly useful for targeting the lumbar region. In some embodiments, the rhNaGlu delivered according to the present invention may achieve an increased enzymatic level or activity in the lumbar region of at least approximately 500 nmol/hr/mg, 600 nmol/hr/mg, 700 nmol/hr/mg, 800 nmol/hr/mg, 900 nmol/hr/mg, 1000 nmol/hr/mg, 1500 nmol/hr/mg, 2000 nmol/hr/mg, 3000 nmol/hr/mg, 4000 nmol/hr/mg, 5000 nmol/hr/mg, 6000 nmol/hr/mg, 7000 nmol/hr/mg, 8000 nmol/hr/mg, 9000 nmol/hr/mg, or 10,000 nmol/hr/mg. Ranges and values intermediate to the above recited ranges and values are also contemplated to be part of the invention.

In general, therapeutic agents (e.g., the rhNaGlu) delivered according to the present invention have sufficiently long half time in CSF and target tissues of the brain, spinal cord, and peripheral organs. In some embodiments, the rhNaGlu delivered according to the present invention may have a half-life of at least approximately 30 minutes, 45 minutes, 60 minutes, 90 minutes, 2 hours, 3 hours, 4 hours, 5 hours, 6 hours, 7 hours, 8 hours, 9 hours, 10 hours, 12 hours, 16 hours, 18 hours, 20 hours, 25 hours, 30 hours, 35 hours, 40 hours, up to 3 days, up to 7 days, up to 14 days, up to 21 days or up to a monthIn some embodiments, the rhNaGlu delivered according to the present invention may retain detectable level or activity in CSF or bloodstream after 12 hours, 24 hours, 30 hours, 36 hours, 42 hours, 48 hours, 54 hours, 60 hours, 66 hours, 72 hours, 78 hours, 84 hours, 90 hours, 96 hours, 102 hours, or a week following administration. Detectable level or activity may be determined using various methods known in the art. Ranges and values intermediate to the above recited ranges and values are also contemplated to be part of the invention.

In certain embodiments, the rhNaGlu delivered according to the present invention achieves a concentration of at least 30μg/mL in the CNS tissues and cells of the subject following administration (e.g., one week, 3 days, 48 hours, 36 hours, 24 hours, 18 hours, 12 hours, 8 hours, 6 hours, 4 hours, 3 hours, 2 hours, 1 hour, 30 minutes, or less, following administration of the pharmaceutical composition to the subject). In certain embodiments, the rhNaGlu delivered according to the present invention achieves a concentration of at least 2 μg/mL, at least 15 μg/mL, at least 1 μg/mL, at least 7 μg/mL, at least 5 μg/mL, at least 2 μg/mL, at least 1 μg/mL or at least 0.5 μg/mL in the targeted tissues or cells of the subject(e.g., brain tissues or neurons) following administration to such subject (e.g., one week, 3 days, 48 hours, 36 hours, 24 hours, 18 hours, 12 hours, 8 hours, 6 hours, 4 hours, 3 hours, 2 hours, 1 hour, 30 minutes, or less following administration of such pharmaceutical compositions to the subject). Ranges and values intermediate to the above recited ranges and values are also contemplated to be part of the invention.

H. Treatment of Sanfilippo Syndrome

Sanfilippo syndrome, or mucopolysaccharidosis III (MPS III), is a rare genetic disorder characterized by the deficiency of enzymes involved in the degradation of glycosaminoglycans (GAG). In the absence of enzyme, partially degraded GAG molecules cannot be cleared from the body and accumulate in lysosomes of various tissues, resulting in progressive widespread somatic dysfunction (Neufeld and Muenzer, 2001).

Four distinct forms of MPS III, designated MPS IIIA, B, C, and D, have been identified. Each represents a deficiency in one of four enzymes involved in the degradation of the GAG heparan sulfate. All forms include varying degrees of the same clinical symptoms, including coarse facial features, hepatosplenomegaly, corneal clouding and skeletal deformities. Most notably, however, is the severe and progressive loss of cognitive ability, which is tied not only to the accumulation of heparan sulfate in neurons, but also the subsequent elevation of the gangliosides GM2, GM3 and GD2 caused by primary GAG accumulation (Walkley 1998).

Mucopolysaccharidosis type TIM (MPS IIIB; Sanfilippo B disease) is an autosomal recessive disorder that is characterized by a deficiency of the enzyme alpha-N-acetyl-glucosaminidase (NaGlu). In the absence of this enzyme, GAG heparan sulfate accumulates in lysosomes of neurons and glial cells, with lesser accumulation outside the brain.

A defining clinical feature of this disorder is central nervous system (CNS) degeneration, which results in loss of, or failure to attain, major developmental milestones. The progressive cognitive decline culminates in dementia and premature mortality. The disease typically manifests itself in young children, and the lifespan of an affected individual generally does not extend beyond late teens to early twenties.

Compositions and methods of the present invention may be used to effectively treat individuals suffering from or susceptible to Sanfilippo B. The terms, “treat” or “treatment,” as used herein, refers to amelioration of one or more symptoms associated with the disease, prevention or delay of the onset of one or more symptoms of the disease, and/or lessening of the severity or frequency of one or more symptoms of the disease.

In some embodiments, treatment refers to partial or complete alleviation, amelioration, relief, inhibition, delaying onset, reducing severity and/or incidence of neurological impairment in a Sanfilippo B patient. As used herein, the term “neurological impairment” includes various symptoms associated with impairment of the central nervous system (e.g., the brain and spinal cord). Symptoms of neurological impairment may include, for example, developmental delay, progressive cognitive impairment, hearing loss, impaired speech development, deficits in motor skills, hyperactivity, aggressiveness and/or sleep disturbances, among others.

Thus, in some embodiments, treatment refers to decreased lysosomal storage (e.g., of GAG) in various tissues. In some embodiments, treatment refers to decreased lysosomal storage in brain target tissues, spinal cord neurons, and/or peripheral target tissues. In certain embodiments, lysosomal storage is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. In some embodiments, lysosomal storage is decreased by at least 1-fold, 2-fold, 3-fold, 4-fold, 5-fold, 6-fold, 7-fold, 8-fold, 9-fold or 10-fold as compared to a control. In some embodiments, lysosomal storage is determined by LAMP-1 staining. Ranges and values intermediate to the above recited ranges and values are also contemplated to be part of the invention.

In some embodiments, treatment refers to reduced vacuolization in neurons (e.g., neurons containing Purkinje cells). In certain embodiments, vacuolization in neurons is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. In some embodiments, vacuolization is decreased by at least 1-fold, 2-fold, 3-fold, 4-fold, 5-fold, 6-fold, 7-fold, 8-fold, 9-fold or 10-fold as compared to a control. Ranges and values intermediate to the above recited ranges and values are also contemplated to be part of the invention.

In some embodiments, treatment refers to increased NaGlu enzyme activity in various tissues. In some embodiments, treatment refers to increased NaGlu enzyme activity in brain target tissues, spinal cord neurons and/or peripheral target tissues. In some embodiments, NaGlu enzyme activity is increased by about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900% 1000% or more as compared to a control. In some embodiments, NaGlu enzyme activity is increased by at least 1-fold, 2-fold, 3 -fold, 4-fold, 5-fold, 6-fold, 7-fold, 8-fold, 9-fold or 10-fold as compared to a control. In some embodiments, increased NaGlu enzymatic activity is at least approximately 10 nmol/hr/mg, 20 nmol/hr/mg, 40 nmol/hr/mg, 50 nmol/hr/mg, 60 nmol/hr/mg, 70 nmol/hr/mg, 80 nmol/hr/mg, 90 nmol/hr/mg, 100 nmol/hr/mg, 150 nmol/hr/mg, 200 nmol/hr/mg, 250 nmol/hr/mg, 300 nmol/hr/mg, 350 nmol/hr/mg, 400 nmol/hr/mg, 450 nmol/hr/mg, 500 nmol/hr/mg, 550 nmol/hr/mg, 600 nmol/hr/mg or more. In some embodiments, NaGlu enzymatic activity is increased in the lumbar region. In some embodiments, increased NaGlu enzymatic activity in the lumbar region is at least approximately 2000 nmol/hr/mg, 3000 nmol/hr/mg, 4000 nmol/hr/mg, 5000 nmol/hr/mg, 6000 nmol/hr/mg, 7000 nmol/hr/mg, 8000 nmol/hr/mg, 9000 nmol/hr/mg, 10,000 nmol/hr/mg, or more. Ranges and values intermediate to the above recited ranges and values are also contemplated to be part of the invention.

In certain embodiments, treatment according to the present invention results in a reduction (e.g., about a 5%, 10%, 15%, 20%, 25%, 30%, 40%, 50%, 55%, 60%, 65%, 70%, 75%, 80%, 90%, 95%, 97.5%, 99% or more reduction) or a complete elimination of the presence, or alternatively the accumulation, of one or more pathological or biological markers which are associated with the NaGlu associated disease. Such reduction or elimination may be particularly evident in the cells and tissues of the CNS (e.g., neurons and oligodendrocytes). For example, in some embodiments, upon administration to a subject the pharmaceutical compositions of the present invention demonstrate or achieve a reduction in the accumulation of the biomarker lysosomal associated membrane protein 1 (LAMP1) in the CNS cells and tissues of the subject (e.g., in the cerebral cortex, cerebellum, caudate nucleus and putamen, white matter and/or thalamus). LAMP1 is a glycoprotein highly expressed in lysosomal membranes and its presence is elevated many patients with a lysosomal storage disorder (Meikle et al., Clin. Chem. (1997) 43:1325-1335). The presence or absence of LAMP 1 in patients (e.g., as determined by LAMP staining) with a lysosomal storage disease therefore may provide a useful indicator of lysosomal activity and a marker for both the diagnosis and monitoring of lysosomal storage diseases.

Accordingly, some embodiments of the present invention relate to methods of reducing or otherwise eliminating the presence or accumulation of one or more pathological or biological markers associated with the NaGlu associated disease. Similarly, some embodiments of the invention relate to methods of increasing the degradation (or the rate of degradation) of one or more pathological or biological markers (e.g., LAMP1) associated with lysosomal storage diseases.

In some embodiments, treatment refers to decreased progression of loss of cognitive ability. In certain embodiments, progression of loss of cognitive ability is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. In some embodiments, treatment refers to decreased developmental delay. In certain embodiments, developmental delay is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. Ranges and values intermediate to the above recited ranges and values are also contemplated to be part of the invention.

In some embodiments, treatment refers to increased survival (e.g., survival time). For example, treatment can result in an increased life expectancy of a patient. In some embodiments, treatment according to the present invention results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 1 10%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with similar disease without treatment. In some embodiments, treatment according to the present invention results in an increased life expectancy of a patient by more than about 6 month, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with similar disease without treatment. In some embodiments, treatment according to the present invention results in long term survival of a patient. As used herein, the term “long term survival” refers to a survival time or life expectancy longer than about 40 years, 45 years, 50 years, 55 years, 60 years, or longer. Ranges and values intermediate to the above recited ranges and values are also contemplated to be part of the invention.

The terms, “improve,” “increase” or “reduce,” as used herein, indicate values that are relative to a control. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with Sanfilippo B, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having Sanfilippo B or having the potential to develop Sanfilippo B. The individual can have residual endogenous NaGlu expression and/or activity, or no measurable activity. For example, the individual having SanB may have NaGlu expression levels that are less than about 30-50%, less than about 25-30%, less than about 20-25%, less than about 15-20%, less than about 10-15%, less than about 5-10%, less than about 0.1-5% of normal NaGlu expression levels. Ranges and values intermediate to the above recited ranges and values are also contemplated to be part of the invention.

In some embodiments, the individual is an individual who has been recently diagnosed with the disease. Typically, early treatment (treatment commencing as soon as possible after diagnosis) is important to minimize the effects of the disease and to maximize the benefits of treatment.

I. Combination Therapies

Recombinant human NaGlu proteins, for instance a recombinant human NaGlu protein containing a sufficient amount of oligosaccharides (e.g., mannose and phosphorylated mannose (i.e., M6P)), can be used alone or in combination to treat NaGlu associated diseases (e.g., Sanfilippo Syndrome B). It should be understood that the recombinant human NaGlu proteins of the invention can be used alone or in combination with an additional procedure, e.g., surgical procedure, or agent, e.g., therapeutic agent, the additional procedure or agent being selected by the skilled artisan for its intended purpose. For instance, the additional procedure or agent can be a therapeutic procedure or agent art-recognized as being useful to treat the disease or condition being treated by the recombinant human NaGlu protein of the present invention. The additional procedure or agent also can be an agent that imparts a beneficial attribute to the therapeutic composition, e.g., an agent which affects the viscosity of the composition.

It should also be understood that the combinations which are included within this invention are those combinations useful for their intended purpose. The agents and procedures set forth below are for illustrative purposes and not intended to be limiting to the present invention. The combinations, which are part of this invention, can be the recombinant human NaGlu proteins of the present invention and at least one additional agent or procedure selected from the lists below. The combination can also include more than one additional agent or procedure, e.g., two or three additional agents if the combination is such that the formed composition can perform its intended function.

The combination therapy can include surgical procedures, gene therapy, or enzyme-replacement therapy. Additionally, the recombinant human NaGlu protein can be coformulated with one or more additional therapeutic agents, e.g., other recombinant proteins or antibodies or drugs capable of preventing or reducing the accumulation of undegraded substrates (e.g., substrate reduction therapy).

In one or more embodiments, the combination therapy can include co-administration with immunosuppresants, as discussed in further detail below. Immunosuppresants such as, but not limited to, antihistamines, corticosteroids, sirolimus, voclosporin, ciclosporin, methotrexate, IL-2 receptor directed antibodies, T-cell receptor directed antibodies, TNF-alpha directed antibodies or fusion proteins (e.g., infliximab, etanercept, or adalimumab), CTLA-4-Ig (e.g., abatacept), anti-OX-40 antibodies can also be administered before, during, or after administration of a recombinant human protein, such as a recombinant human NaGlu protein, for example, if an anaphylactic reaction or adverse immune response is expected or experienced by a patient.

J. Immunogenicity

The pharmaceutical compositions of the present invention are characterized by their tolerability. As used herein, the terms “tolerable” and “tolerability” refer to the ability of the pharmaceutical compositions of the present invention to not elicit an adverse reaction in the subject to whom such composition is administered, or alternatively not to elicit a serious adverse reaction in the subject to whom such composition is administered. In some embodiments, the pharmaceutical compositions of the present invention are well tolerated by the subject to whom such compositions is administered.

Generally, administration of a rhNaGlu protein according to the present invention does not result in severe adverse effects in the subject. As used herein, severe adverse effects induce, but are not limited to, substantial immune response, toxicity, or death. As used herein, the term “substantial immune response” refers to severe or serious immune responses, such as adaptive T-cell immune responses.

Thus, in many embodiments, inventive methods according to the present invention do not involve concurrent immunosuppressant therapy (i.e., any immunosuppressant therapy used as pre-treatment/pre-conditioning or in parallel to the method). In some embodiments, inventive methods according to the present invention do not involve an immune tolerance induction in the subject being treated. In some embodiments, inventive methods according to the present invention do not involve a pre-treatment or preconditioning of the subject using T-cell immunosuppressive agent.

However, in some embodiments, a subject mounts an immune response after being administered the rhNaGlu of the invention. Thus, in some embodiments, it may be useful to render the subject receiving the rhNaGlu of the invention tolerant to the enzyme replacement therapy. Immune tolerance may be induced using various methods known in the art. For example, an initial 30-60 day regimen of a T-cell immunosuppressive agent such as cyclosporin A (CsA) and an antiproliferative agent, such as, azathioprine (Aza), combined with weekly intravenous or intrathecal infusions of low doses of a desired replacement enzyme may be used.

Any immunosuppressant agent known to the skilled artisan may be employed together with a combination therapy of the invention. Such immunosuppressant agents include but are not limited to cyclosporine, FK506, rapamycin, CTLA4-Ig, and anti-TNF agents such as etanercept (see e.g., Moder, 2000, Ann. Allergy Asthma Immunol. 84, 280-284; Nevins, 2000, Curr. Opin. Pediatr. 12, 146-150; Kurlberg et al., 2000, Scand. J. Immunol. 51, 224-230; Ideguchi et al., 2000, Neuroscience 95, 217-226; Potteret al., 1999, Ann. N.Y. Acad. Sci. 875, 159-174; Slavik et al., 1999, Immunol. Res. 19, 1-24; Gaziev et al., 1999, Bone Marrow Transplant. 25, 689-696; Henry, 1999, Clin. Transplant. 13, 209-220; Gummert et al., 1999, J. Am. Soc. Nephrol. 10, 1366-1380; Qi et al., 2000, Transplantation 69, 1275-1283). The anti-IL2 receptor (a-subunit) antibody daclizumab (e.g., Zenapax™), which has been demonstrated effective in transplant patients, can also be used as an immunosuppressant agent (see e.g., Wiseman et al., 1999, Drugs 58, 1029-1042; Beniaminovitz et al., 2000, N. Engl J. Med. 342, 613-619; Ponticelli et al., 1999, Drugs R. D. 1, 55-60; Berard et al., 1999, Pharmacotherapy 19, 1 127-1 137; Eckhoff et al., 2000, Transplantation 69, 1867-1872; Ekberg et al., 2000, Transpl. Int. 13, 151-159). Additional immunosuppressant agents include but are not limited to anti-CD2 (Branco et al., 1999, Transplantation 68, 1588-1596; Przepiorka et al., 1998, Blood 92, 4066-4071), anti-CD4 (Marinova-Mutafchieva et al., 2000, Arthritis Rheum. 43, 638-644; Fishwild et al., 1999, Clin. Immunol. 92, 138-152), and anti-CD40 ligand (Hong et al., 2000, Semin. Nephrol. 20, 108-125; Chirmule et al., 2000, J. Virol. 74, 3345-3352; Ito et al., 2000, J. Immunol. 164, 1230-1235).

In other embodiments, the invention includes methods comprising co-administration of the NaGlu proteins of the present invention with agents which decrease or suppress an immune response to the NaGlu protein, e.g., immunosuppresants. Immunosuppresants such as, but not limited to, antihistamines, corticosteroids, sirolimus, voclosporin, ciclosporin, methotrexate, IL-2 receptor directed antibodies, T-cell receptor directed antibodies, TNF-alpha directed antibodies or fusion proteins (e.g., infliximab, etanercept, or adalimumab), CTLA-4-Ig (e.g., abatacept), anti-OX-40 antibodies can also be administered before, during, or after administration of a recombinant human protein, such as a recombinant human NaGlu protein, for example, if an anaphylactic reaction or adverse immune response is expected or experienced by a patient.

In one embodiment, the invention provides for a pretreatment procedure to minimize or prevent any potential anaphylactic reactions that can be incurred by administration of the recombinant protein in accordance with the invention. In one embodiment, to prevent a potential anaphylactic reaction, an H-1 receptor antagonist, also known as an antihistamine (e.g., diphenhydramine) is administered to the patient. In one embodiment, the H-1 receptor antagonist is administered in a dose of about 1 mg to about 10 mg per kilogram of body weight. For example, an antihistamine can be administered in a dose of about 5 mg per kilogram. In one embodiment, the antihistamine is administered in a dose of between about 0.1 mg and about 10 mg per kilogram of body weight. In one embodiment, the antihistamine is administered in a dose between about 1 mg and about 5 mg per kilogram of body weight. For example the dose can be 1 mg, 2 mg, 3 mg, 4 mg, or 5 mg per kilogram of body weight. The antihistamine can be administered by any useful method. In one embodiment, the antihistamine is administered intravenously. In another embodiment, the antihistamine is administered in pharmaceutically acceptable capsules.

Administration of the antihistamine can be prior to the administration of the recombinant NaGlu in accordance with the invention. In one embodiment, the H-1 receptor antagonist is administered about 10 to about 90 minutes, for example, about 30 to about 60 minutes prior to the administration of recombinant NaGlu. The H-1 receptor antagonist can be administered using an ambulatory system connected to a vascular access port. In one embodiment, the antihistamine is administered about 90 minutes prior to the administration of recombinant NaGlu. In one embodiment, the antihistamine is administered between about 10 and about 60 minutes prior to the administration of recombinant NaGlu. In another embodiment, the antihistamine is administered between about 20 and about 40 minutes prior to administering recombinant NaGlu. For example, the antihistamine can be administered 20, 25, 30, 35, or 40 minutes prior to the administration of recombinant NaGlu.

In one embodiment, the antihistamine administered is diphenhydramine. Any useful antihistamine can be used. Such antihistamines include, without limitation, clemastine, doxylamine, loratidine, desloratidine, fexofenadine, pheniramine, cetirizine, ebastine, promethazine, chlorpheniramine, levocetirizine, olopatadine, quetiapine, meclizine, dimenhydrinate, embramine, dimethidene, and dexchloropheniramine.

In another embodiment, with reference to intravenous infusion, the potential for anaphylactic reactions can be reduced by administering the infusions using a ramp-up protocol. In this context, a ramp-up protocol refers to slowly increasing the rate of the infusion over the course of the infusion in order to desensitize the patient to the infusion of the medication.

K. Administration

The methods of the present invention contemplate single as well as multiple administrations of a therapeutically effective amount of the rhNaGlu of the invention described herein. The rhNaGlu of the invention can be administered at regular intervals, depending on the nature, severity and extent of the subject's condition. In some embodiments, a therapeutically effective amount of the rhNaGlu protein of the present invention may be administered intravenously (e.g., via intravenous infusion or injection) or intrathecally, periodically at regular intervals (e.g., once every year, once every six months, once every five months, once every three months, bimonthly (once every two months), monthly (once every month), biweekly (once every two weeks) or weekly.

The frequency of administration may be constant, or may change over time, becoming more or less frequent. As a nonlimiting example, administration of a first therapeutically effective amount of the rhNaGlu weekly for a period of time can be followed by the administration of a therapeutically effective amount of the rhNaGlu every other week, every three weeks, or every four weeks, for a second period of time.

The duration of treatment can be fixed, or it can be life-long. Treatment duration can be, for example, 12 weeks, 24 weeks, 52 weeks, 2 years, 3 years, and longer. Because the treatment with rhNaGlu replaces a deficient enzyme in the subject, it is expected that the duration of treatment is life-long. However, the both the frequency and the dose of rhNaGlu may vary during the duration of treatment. For example, the therapeutically effective dose of rhNaGlu may be higher or lower at different points during treatment, and dose administration may be less frequent or more frequent at different points during treatment.

The treatment method may include multiple administrations of a therapeutically effective amount of rhNaGlu over an extended period of time after an initial treatment with rhNaGlu or other treatment modality has successfully lowered levels of HS in serum, CNS or other target tissue or cells of the subject, e.g., for a time exceeding one week, two weeks, one month, two months, three months, four months, five months, six months, nine months, or one year. rhNaGlu can be administered at regular intervals, depending on the nature, severity and extent of the subject's condition. In some embodiments, e.g., once the initial therapeutic effect has been achieved, an amount of rhNaGlu sufficient to maintain therapeutically effective levels of rhNaGlu can be administered systemically (e.g., intravenously) periodically at regular intervals (e.g., once every year, once every six months, once every five months, once every three months, bimonthly (once every two months), monthly (once every month), biweekly (once every two weeks) or weekly.

rhNaGlu can be administered therapeutically or prophylactically, or both. rhNaGlu can be administered to the subject, alone or in combination with other therapeutic modalities as described herein.

In some embodiments, intrathecal administration may be used in conjunction with other routes of administration (e.g., intravenous, subcutaneously, intramuscularly, parenterally, trans dermally, or transmucosally (e.g., orally or nasally)). In some embodiments, those other routes of administration (e.g., intravenous administration) may be performed no more frequent than biweekly, monthly, once every two months, once every three months, once every four months, once every five months, once every six months, annually administration.

As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition). For example, a therapeutically effective amount may be an amount sufficient to achieve a desired therapeutic and/or prophylactic effect, such as an amount sufficient to modulate lysosomal enzyme receptors or their activity to thereby treat such lysosomal storage disease or the symptoms thereof (e.g., a reduction in or elimination of the presence or incidence of “zebra bodies” or cellular vacuolization following the administration of the compositions of the present invention to a subject). Generally, the amount of a therapeutic agent (e.g., the rhNaGlu of the invention) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

In some embodiments, the therapeutically effective dose ranges from about 0.005 mg/kg body weight to 500 mg/kg body weight, e.g., from about 0.005 mg/kg body weight to 400 mg/kg body weight, from about 0.005 mg/kg body weight to 300 mg/kg body weight, from about 0.005 mg/kg body weight to 200 mg/kg body weight, from about 0.005 mg/kg body weight to 100 mg/kg body weight, from about 0.005 mg/kg body weight to 90 mg/kg body weight, from about 0.005 mg/kg body weight to 80 mg/kg body weight, from about 0.005 mg/kg body weight to 70 mg/kg body weight, from about 0.005 mg/kg body weight to 60 mg/kg body weight, from about 0.005 mg/kg body weight to 50 mg/kg body weight, from about 0.005 mg/kg body weight to 40 mg/kg body weight, from about 0.005 mg/kg body weight to 30 mg/kg body weight, from about 0.005 mg/kg body weight to 25 mg/kg body weight, from about 0.005 mg/kg body weight to 20 mg/kg body weight, from about 0.005 mg/kg body weight to 15 mg/kg body weight, from about 0.005 mg/kg body weight to 10 mg/kg bra body in weight. Ranges and values intermediate to the above recited ranges and values (e.g., 10-50 mg/kg, 1-5 mg/kg, 2-8 mg/kg, 5-10 mg/kg, 0.1-10 mg/kg, 0.3-30 mg/kg, 0.3-50 mg/kg, 0.5-10 mg/kg, 5-30 mg/kg, or 6-27 mg/kg) are also contemplated to be part of the invention.

In some embodiments, the therapeutically effective dose is greater than or at least about 0.1 mg/kg body weight, greater than or at least about 0.2 mg/kg body weight, greater than or at least about 0.3 mg/kg body weight, greater than or at least about 0.4 mg/kg body weight, greater than or at least about 0.5 mg/kg body weight, greater than or at least about 1.0 mg/kg body weight, greater than or at least about 3 mg/kg body weight, greater than or at least about 5 mg/kg body weight, greater than or at least about 6 mg/kg body weight, greater than or at least about 7 mg/kg body weight greater than or at least about 10 mg/kg body weight, greater than or at least about 15 mg/kg body weight, greater than or at least about 20 mg/kg body weight, greater than or at least about 30 mg/kg body weight, greater than or at least about 40 mg/kg body weight, greater than or at least about 50 mg/kg body weight, greater than or at least about 60 mg/kg body weight, greater than or at least about 70 mg/kg body weight, greater than about or at least 80 mg/kg body weight, greater than or at least about 90 mg/kg body weight, greater than or at least about 100 mg/kg body weight. Ranges and values intermediate to the above recited ranges and values are also contemplated to be part of the invention.

In some embodiments, the therapeutically effective dose may also be defined by mg/kg brain weight. As one skilled in the art would appreciate, the brain weights and body weights can be correlated (see, e.g., Dekaban AS. “Changes in brain weights during the span of human life: relation of brain weights to body heights and body weights,” Ann Neurol 1978; 4:345-56).

In some embodiments, the therapeutically effective dose may also be defined by mg/15 cc of CSF. As one skilled in the art would appreciate, therapeutically effective doses based on brain weights and body weights can be converted to mg/15 cc of CSF. For example, the volume of CSF in adult humans is approximately 150 mL (Johanson CE, et al. “Multiplicity of cerebrospinal fluid functions: New challenges in health and disease,” Cerebrospinal Fluid Res. 2008 May 14;5: 10). Therefore, single dose injections of 0.1 mg to 50 mg protein to adults would be approximately 0.01 mg/15 cc of CSF (0.1 mg) to 5.0 mg/15 cc of CSF (50 mg) doses in adults.

In some embodiments, rhNaGlu is intravenously administered at a first dose periodically for a first period, and intravenously at a second dose periodically for a second period. In one embodiment, there can be a washout period between the first period and the second period that the patient does not receive any rhNaGlu therapy. It is to be further understood that for any particular subject, specific dosage regimens should be adjusted over time according to the individual need and the professional judgment of the person administering or supervising the administration of the enzyme replacement therapy and that dosage ranges set forth herein are exemplary only and are not intended to limit the scope or practice of the claimed invention.

The therapeutic methods of the present invention encompass any systemic route of administration which facilitates the uptake or transport of rhNaGlu into the pertinent organs and tissues. In preferred embodiments, rhNaGlu may be administered to the patient intravenously (e.g., via intravenous injection, intravenous infusion) and in an amount effective to cross the blood brain barrier (BBB) of the subject with NaGlu deficiency. The methods of treatment described herein effectively deliver the rhNaGlu proteins to various tissues and neurons or cells of the central nervous system (CNS) in patients with MPS IIIB. Furthermore, the methods of the present invention deliver sufficient amounts of the rhNaGlu proteins to the blood stream and various peripheral organs and tissues.

### II. Recombinant Human NaGlu

The methods described herein include the systemic, e.g., intravenous, administration of compositions of rhNaGlu (amino acid sequence 24-743 set forth in SEQ ID NO:1) having patterns of glycosylation that confer an increased cellular uptake and an increased subcellular activity which are particularly useful for therapy, for example, in the treatment of Sanfilippo Syndrome B (mucopolysaccharidosis (MPS) IIIB).

Human NaGlu Amino Acid Sequence (Signal Peptide: aa1-23, Underlined)

| (SEQ ID NO: 1) | |
|---|---|
| MEAVAVAAAV GVLLLAGAGG AAGDEAREAA AVRALVARLL GPGPAADFSV SVERALAAKP | 60 |
| GLDTYSLGGG GAARVRVRGS TGVAAAAGLH RYLRDFCGCH VAWSGSQLRL PRPLPAVPGE | 120 |
| LTEATPNRYR YYQNVCTQSY SFVWWDWARW EREIDWMALN GINLALAWSG QEAIWQRVYL | 180 |
| ALGLTQAEIN EFFTGPAFLA WGRMGNLHTW DGPLPPSWHI KQLYLQHRVL DQMRSFGMTP | 240 |
| VLPAFAGHVP EAVTRVFPQV NVTKMGSWGH FNCSYSCSFL LAPEDPIFPI IGSLFLRELI | 300 |
| KEFGTDHIYG ADTFNEMQPP SSEPSYLAAA TTAVYEAMTA VDTEAVWLLQ GWLFQHQPQF | 360 |
| WGPAQIRAVL GAVPRGRLLV LDLFAESQPV YTRTASFQGQ PFIWCMLHNF GGNHGLFGAL | 420 |
| EAVNGGPEAA RLFPNSTMVG TGMAPEGISQ NEVVYSLMAE LGWRKDPVPD LAAWVTSFAA | 480 |
| RRYGVSHPDA GAAWRLLLRS VYNCSGEACR GHNRSPLVRR PSLQMNTSIW YNRSDVFEAW | 540 |
| RLLLTSAPSL ATSPAFRYDL LDLTRQAVQE LVSLYYEEAR SAYLSKELAS LLRAGGVLAY | 600 |
| ELLPALDEVL ASDSRFLLGS WLEQARAAAV SEAEADFYEQ NSRYQLTLWG PEGNILDYAN | 660 |
| KQLAGLVANY YTPRWRLFLE ALVDSVAQGI PFQQHQFDKN VFQLEQAFVL SKQRYPSQPR | 720 |
| GDTVDLAKKI FLKYYPRWVA GSW | 743 |

In some aspects, the composition can be an isolated mixture of rhNaGlu comprising the amino acid sequence 24-743 of SEQ ID NO:1. In one embodiment, the mixture contains a sufficient amount of rhNaGlu having at least one glycan structure that contains phosphorylated mannose (e.g., M6P or bis-M6P) or mannose such that the rhNaGlu containing M6P or mannose is internalized into a human cell deficient in NaGlu and restores at least 50% of NaGlu activity observed in a wild-type human cell of the same type that actively expresses endogenous NaGlu. In one aspect, at least 10%, 20%, 30%, 40%, 50%, 60%, 70%, 80%, 90%, 91%, 92%, 93% 94%, 95%, 96%, 97%, 98% or 99% of rhNaGlu in the mixture contains at least one glycan structure having phosphorylated mannose and/or mannose. In one embodiment, at least 10% of rhNaGlu in the mixture contains at least one glycan structure having phosphorylated mannose and/or mannose. In one embodiment, at least 20% of rhNaGlu in the mixture contains at least one glycan structure having phosphorylated mannose and/or mannose. In one embodiment, at least 30% of rhNaGlu in the mixture contains at least one glycan structure having phosphorylated mannose and/or mannose. In one embodiment, at least 30% of rhNaGlu in the mixture contains at least one glycan structure having phosphorylated mannose and/or mannose. In one embodiment, at least 40% of rhNaGlu in the mixture contains at least one glycan structure having phosphorylated mannose and/or mannose. In one embodiment, at least 50% of rhNaGlu in the mixture contains at least one glycan structure having phosphorylated mannose and/or mannose. In one embodiment, at least 60% of rhNaGlu in the mixture contains at least one glycan structure having phosphorylated mannose and/or mannose.

In some aspects, rhNaGlu contains one or more N-linked glycan structure. The rhNaGlu contains at least one phosphorylated mannose (M6P or bis-M6P) which allows the protein to be taken up into a human cell, including but not limited to, a skin fibroblast, an endothelial, a neuronal cell, hepatocyte, a macrophage or any cell that expresses M6P receptor on the cell surface via M6P receptor-mediated endocytosis. In one embodiment, the rhNaGlu contains at least one mannose (Man). In another embodiment, the rhNaGlu contains at least one N-acetylglucosamine (GlcNAc).

In some aspects, the rhNaGlu contains a glycan structure comprising a phosphorylated mannose (M6P or bis-M6P). It is to be understood that M6P referred herein can include mono- and bis-phosphorylated mannose. M6P can include mono- and bis-phosphorylated mannose. In one embodiment, the M6P is present at a concentration that is about 1, about 2, about 3, about 4, about 5 or about 6 mole(s) per mole of protein. In one embodiment, the rhNaGlu contains M6P at a concentration that is about 2, about 3, about 4, or about 5 moles per mole of protein. In one embodiment, the rhNaGlu contains M6P at a concentration that is about 2 moles per mole of protein. In one embodiment, the rhNaGlu contains M6P at a concentration that is about 3 moles per mole of protein. In one embodiment, the rhNaGlu contains M6P at a concentration that is about 4 moles per mole of protein. In one embodiment, the rhNaGlu contains M6P at a concentration that is about 5 moles per mole of protein. In one embodiment, the rhNaGlu contains M6P at a concentration that is about 6 moles per mole of protein.

In some aspects, the rhNaGlu contains a sufficient amount of M6P or bis-M6P for cellular uptake into a human cell having a M6P receptor on the cell surface via M6P receptor-mediated endocytosis. In one embodiment, a sufficient amount of M6P for uptake into a human cell is about 1, 2, 3, 4, 5 or 6 moles per mole of protein. The rhNaGlu can be internalized into a human cell deficient in NaGlu such that the internalized protein fully (100% or more) restores a normal level of NaGlu activity in the human cell deficient in NaGlu. In one embodiment, the internalized rhNaGlu protein fully restores a normal level of NaGlu activity in the human cell at a concentration that is at least 0.5, 0.6, 0.7, 0.8, 0.9 or 1.0 μg/mL. In one embodiment, the internalized protein fully restores a normal level of NaGlu activity in the human cell deficient in NaGlu at a concentration that is at least 2, 3, 4, 5, 6, 7, 8, 9 or 10 μg/mL. In one embodiment, the internalized rhNaGlu fully restores a normal level of NaGlu activity in the human cell at a concentration that is at least 20, 30, 40, 50, 60, 70, 80, 90 or 100 μg/mL. As used herein, the normal level of NaGlu activity is a level of NaGlu activity measured in a wild-type human cell of the same type that actively expresses a normal NaGlu enzyme.

In some aspects, the rhNaGlu can be internalized into a human cell deficient in NaGlu such that the protein restores at least about 50%, about 60%, about 70%, about 80%, about 90% or about 95% of NaGlu activity of a normal human cell of the same type.

In some embodiments, the rhNaGlu can be internalized into a human cell deficient in NaGlu such that the internalized rhNaGlu provides a higher enzymatic activity than that observed in a normal human cell of the same type.

In one embodiment, the rhNaGlu is internalized into a human cell deficient in NaGlu such that the internalized rhNaGlu provides about 2, about 3, about 4, about 5, about 6, about 7, about 8, about 9 and about 10-fold higher activity than that observed in a normal human cell of the same type. In one embodiment, the rhNaGlu is internalized into a human cell deficient in NaGlu such that the internalized rhNaGlu provides about 15, about 20, about 25, about 30, about 40, about 50, about 60, about 70, about 80, about 90 or about 100-fold higher activity than that observed in a normal human cell.

In one embodiment, the human cell deficient in NaGlu is any human cell deficient in NaGlu that expresses one or more M6P receptors on the cell surface. In one embodiment, the human cell deficient in NaGlu is a human mucopolysaccharidosis (MPS) IIIB fibroblast that accumulates heparan sulfate. In one embodiment, the human cell deficient in NaGlu is a hepatocyte. In one embodiment, the human cell deficient in NaGlu is a neuronal cell, or a glial cell. In one embodiment, the human cell deficient in NaGlu is an endothelial cell. In one embodiment, the human cell deficient in NaGlu is a macrophage.

In some aspects, uptake of rhNaGlu into a human cell is inhibited by the presence of about 1, about 2, about 3, about 4, about 5, about 6, about 7, about 8, about 9 or about 10 mM of competing M6P monosaccharide. In some aspects, the cellular uptake of rhNaGlu is inhibited by the presence of about 0.1, about 0.2, about 0.3, about 0.4, about 0.5, about 0.6, about 0.7, about 0.8, about 0.9 or about 1.0 mM of M6P monosaccharide. In one embodiment, the cellular uptake of rhNaGlu is inhibited by the presence of about 0.01, about 0.02, about 0.03, about 0.04, about 0.05, about 0.06, about 0.07, about 0.08, or about 0.09 mM of M6P monosaccharide.

In some aspects, the rhNaGlu contains mannose in its glycan structures at a concentration that is about 17, 18, 19, 20, 21, 22, 23, 24, 25, 26, 27, 28, 29, 30, 31, 32, 33, 34 or 35 moles per mole of protein. In one embodiment, the rhNaGlu has mannose at a concentration that is about 20, 21, 22, 23, 24, 25, 26, 27, 28, 29 or 30 moles per mole of protein. The rhNaGlu contains mannose at a concentration that is about 22, 23, 24, 25, 26, 27 or 28 moles per mole of protein. The rhNaGlu contains mannose at a concentration that is about 24 moles per mole of protein. The rhNaGlu protein contains mannose at a concentration that is about 25 moles per mole of protein. The rhNaGlu contains mannose at a concentration that is about 26 moles per mole of protein. The rhNaGlu contains mannose at a concentration that is about 27 moles per mole of protein. In one embodiment, the rhNaGlu has mannose at a concentration that is between about 20 and about 30 moles per mole of protein.

In some aspects, the rhNaGlu comprises N-acetylglucosamine (GlcNAc). In one embodiment, the rhNaGlu contains GlcNAc at a concentration that is between about 28 and about 42 moles per mole of protein. In one embodiment, the NaGlu protein has GlcNAc at a concentration that is between about 30 and about 40 moles per mole of protein. In one embodiment, the NaGlu protein comprises GlcNAc at a concentration that is between about 32 and about 38 moles per mole of protein. In one embodiment, the NaGlu protein comprises GlcNAc at a concentration that is between about 34 and about 36 moles per mole of protein. In one embodiment, the NaGlu protein has GlcNAc at a concentration that is about 35 moles per mole of protein. In one embodiment, the rhNaGlu protein contains GlcNAc at a concentration that is about 30, 31, 32, 33, 34, 35, 36, 37, 38, 39 or 40 moles per mole of protein.

In some aspects, the rhNaGlu contains N-acetylgalactosamine (GalNAc) and/or galactose (Gal). The presence of the GalNAc and Gal typically indicates that the NaGlu may contain one or more O-linked glycan structures which are added to the protein in the Golgi compartment. Accordingly, the present invention optionally includes a composition comprising a recombinant human NaGlu that contains one or more 0-linked glycan structure.

In one embodiment, the rhNaGlu contains galactose at a concentration that is about 1, 2, 3, 4, 5, 6 or 7 moles per mole of protein. In one embodiment, the rhNaGlu has galactose at a concentration that is about 2, 3, 4, 5 or 6 moles per mole of protein. In one embodiment, the rhNaGlu has galactose at a concentration that is about 3 moles per mole of protein. In one embodiment, the rhNaGlu has galactose at a concentration that is about 4 moles per mole of protein.

In one embodiment, the NaGlu comprises at least one GalNAc molecule per mole of protein. In one embodiment, the NaGlu comprises GalNAc at a concentration that is about 1 or 2 moles per mole of protein.

In one embodiment, the NaGlu contains no fucose. In yet another embodiment, the NaGlu contains no glucose. In yet another embodiment, rhNaGlu contains neither fucose nor glucose.

In certain aspects, the rhNaGlu is N-glycosylated at 1, 2, 3, 4, 5, 6, 7, 8, 9, or 10 sites with an N-glycan that comprises a bisecting sugar residue. In one embodiment, the bisecting sugar residue is GlcNAc. In a population of such proteins at least 5%, 10%, 20%, 30%, 40%, 50%, 60%, 70%, 80%, 90%, 95%, 98%, or at least 99%, or 100% of the proteins comprise at least 1, 2, 3, 4, 5, 6, 7, 8, 9, or at least 10 N-glycans comprising a bisecting sugar, such as a bisecting GlcNAc. In another embodiment, the rhNaGlu further comprises at least 1, 2, 3, 4, 5, 6, 7, 8, 9, or at least 10 N-glycans that are high mannose (such as bi-, tri, or tetrantennary high mannose glycans). In yet another embodiment, in a population of such proteins, at least 5%, 10%, 20%, 30%, 40%, 50%, 60%, 70%, 80%, 90%, 95%, 98%, or at least 99%, or 100% of the proteins comprise at least 1, 2, 3, 4, 5, 6, 7, 8, 9, or at least 10 N-glycans that is high mannose (such as biantennary, triantennary, or tetraantennary). In certain embodiments, at least 80%, 85%, 90%, 95%, 98%, 99%, or 100% of the N-glycans of any such proteins are not sialylated (i.e., lack neuraminic acid). In certain specific aspects, one, two, three, four, or all terminal sugar residues of the N-glycan with a bisecting GlcNAc are GlcNAc. The N-glycan with a bisecting GlcNAc can, for example, be Structure 12 wherein at least one of the GlcNAc's to the left of the curly bracket (Table A) is attached to the mannose closest to the reducing end (i.e., the end at the right of the structure as shown in Table A) of the glycan.

In certain aspects, the rhNaGlu is N-glycosylated at 1, 2, 3, 4, 5, 6, 7, 8, 9, or 10 sites with a complex-type N-glycan that comprises a bisecting sugar. In one embodiment, the bisecting sugar residue is GlcNAc. In a population of such proteins at least 5%, 10%, 20%, 30%, 40%, 50%, 60%, 70%, 80%, 90%, 95%, 98%, or at least 99%, or 100% of the proteins comprise at least 1, 2, 3, 4, 5, 6, 7, 8, 9, or at least 10 complex-type N-glycans comprising a bisecting sugar, such as a bisecting GlcNAc. In another embodiment, the rhNaGlu further comprises at least 1, 2, 3, 4, 5, 6, 7, 8, 9, or at least 10 N-glycans that are high mannose (such as bi-, tri, or tetrantennary high mannose glycans). In yet another embodiment, in a population of such proteins at least 5%, 10%, 20%, 30%, 40%, 50%, 60%, 70%, 80%, 90%, 95%, 98%, or at least 99%, or 100% of the proteins comprise at least 1, 2, 3, 4, 5, 6, 7, 8, 9, or at least 10 N-glycans that is high mannose (such as biantennary, triantennary, or tetraantennary). In certain embodiments, at least 80%, 85%, 90%, 95%, 98%, 99%, or 100% of the N-glycans of any such proteins are not sialylated. In certain specific aspects, one, two, three, four, or all terminal sugar residues of the N-glycan with a bisecting GlcNAc are GlcNAc. The N-glycan with a bisecting GlcNAc can, for example, be Structure 12 wherein at least one of the GlcNAc's to the left of the curly bracket (Table A) is attached to the mannose closest to the reducing end (i.e., the end at the right of the structure as shown in Table A) of the glycan.

In certain aspects, the rhNaGlu is N-glycosylated at 1, 2, 3, 4, 5, 6, 7, 8, 9, or 10 sites with a hybrid-type N-glycan that comprises a bisecting sugar residue. In one embodiment, the bisecting sugar residue is GlcNAc. In a population of such proteins at least 5%, 10%, 20%, 30%, 40%, 50%, 60%, 70%, 80%, 90%, 95%, 98%, or at least 99%, or 100% of the proteins comprise at least 1, 2, 3, 4, 5, 6, 7, 8, 9, or at least 10 hybrid-type N-glycans comprising a bisecting sugar, such as a bisecting GlcNAc. In another embodiment, the rhNaGlu further comprises at least 1, 2, 3, 4, 5, 6, 7, 8, 9, or at least 10 N-glycans that are high mannose (such as bi-, tri, or tetrantennary high mannose glycans). In yet another embodiment, in a population of such proteins at least 5%, 10%, 20%, 30%, 40%, 50%, 60%, 70%, 80%, 90%, 95%, 98%, or at least 99%, or 100% of the proteins comprise at least 1, 2, 3, 4, 5, 6, 7, 8, 9, or at least 10 N-glycan that is high mannose (such as biantennary, triantennary, or tetraantennary). In certain embodiments, at least 80%, 85%, 90%, 95%, 98%, 99%, or 100% of the N-glycans of any such proteins are not sialylated. In certain specific aspects, one, two, three, or four terminal sugar residues of the N-glycan with a bisecting GlcNAc are GlcNAc. The N-glycan with a bisecting GlcNAc can, for example, be Structure 12 wherein at least one of the GlcNAc's to the left of the curly bracket (Table A) is attached to the mannose closest to the reducing end (i.e., the end at the right of the structure as shown in Table A) of the glycan.

In more specific aspects, at least 5%, 10%, 15%, 20%, 25%, 30%, 35%, 40%, 45%, 50%, 55%, 60%, 65%, 70%, 75%, 80%, 85%, 90%, 95%, or all of the N-glycosylation is mannosidase resistant despite the presence of mannose residues in the glycosylation. In certain embodiments, a glycan is mannosidase resistant if the digestion of a population of such glycans requires at least 2-fold, 3-fold, 4-fold, 5-fold, 10-fold 25-fold, 50-fold, 75-fold, or 100-fold more mannosidase enzyme activity than the digestion of an equal amount of Structure 1 of Table A.

In certain aspects, the rhNaGlu is capable of binding to Phytohaemagglutinin-E (PHA-E). In certain more specific aspects, at least 5%, 10%, 15%, 20%, 25%, 30%, 35%, 40%, 45%, 50%, 55%, 60%, 65%, 70%, 75%, 80%, 85%, 90%, 95%, or all of the proteins in a population of proteins bind to PHA-E.

In certain aspects, the rhNaGlu is N-glycosylated, wherein at least 25%, 50%, 75%, 80%, 90%, 95%, or at least 98% of the glycosylation structures are complex type structures with terminal GlcNAc.

In certain aspects, the rhNaGlu is N-glycosylated on 1, 2, 3, 4, 5, 6, 7, 8, 9, or 10 N-glycosylation sites, wherein at least 25%, 50%, 75%, 80%, 90%, 95%, 98%, or at least 99%, or 100% of the proteins in a population of proteins comprise complex-type N-glycans. More specifically, the rhNaGlu is N-glycosylated on 1, 2, 3, 4, 5, 6, 7, 8, 9, or 10 N-glycosylation sites, wherein at least 25%, 50%, 75%, 80%, 90%, 95%, 98%, or at least 99%, or 100% of the proteins in a population of proteins comprise complex-type N-glycans and wherein the proteins are not sialylated, or carry at most 1, 2, 3, 4, or 5 sialic acid residues per protein.

In certain aspects, the rhNaGlu is characterized by:

a) low sialic acid (i.e., low neuraminic acid) content (i.e., the protein carries at most 1, 2, 3, 4, or 5 sialic acid residues) or absence of sialic acids; and

b) one or more of the following:

(i) at least 1, 2, 3, 4, 5, 6, 7, 8, 9, or 10 mannose-6-phosphate residues,

(ii) at least 1, 2, 3, 4, 5, 6, 7, 8, 9, or 10 bisecting GlcNAc residues,

(iii) at least 1, 2, 3, 4, 5, 6, 7, 8, 9, or 10 high mannose structures, and

(iv) one or more of Structure 1 to 20 (see Table A).

With respect to a population of proteins, at least 5%, 10%, 20%, 30%, 40%, 50%, 60%, 70%, 80%, 90%, 95%, 98%, or at least 99%, or 100% of the proteins are characterized by:

a) low sialic acid (i.e., low neuraminic acid) content (i.e., the protein carries at most 1, 2, 3, 4, or 5 sialic acid residues) or absence of sialic acids; and

b) one or more of the following:

(i) at least 1, 2, 3, 4, 5, 6, 7, 8, 9, or 10 mannose-6-phosphate residues,

(ii) at least 1, 2, 3, 4, 5, 6, 7, 8, 9, or 10 bisecting GlcNAc residues,

(iii) at least 1, 2, 3, 4, 5, 6, 7, 8, 9, or 10 high mannose structures, and

(iv) one or more of Structure 1 to 20 (see Table A).

In certain aspects, the rhNaGlu is N-glycosylated at one or more N-glycosylation sites. Structures of these N-glycans can be selected from Structures 1 to 20 shown in Table A below.

In certain aspects, the rhNaGlu is N-glycosylated on 1, 2, 3, 4, 5, 6, 7, 8, 9, or 10 N-glycosylation sites, wherein at least 25%, 50%, 75%, 80%, 90%, 95%, 98%, or at least 99%, or 100% of the proteins in a population of proteins comprise one or more N-glycans selected from Structure 1 to Structure 20 (Table A).

In certain aspects, the rhNaGlu is N-glycosylated on 1, 2, 3, 4, 5, 6, 7, 8, 9, or 10 N-glycosylation sites, wherein at least 25%, 50%, 75%, 80%, 90%, 95%, 98%, or at least 99%, or 100% of the proteins in a population of proteins comprise one or more N-glycans selected from Structure 8, Structure 12, Structure 15, Structure 16, and Structure 17 (Table A).

In specific aspects, the rhNaGlu carries one or more of structure 8, 12, 15, 16, and 17 of Table A. In more specific aspects, at least 5%, 10%, 15%, 20%, 25%, 30%, 35%, 40%, 45%, 50%, 55%, 60%, 65%, 70%, 75%, 80%, 85%, 90%, 95%, or all of the N-glycosylation sites of a population of the protein comprise one or more of Structure 8, Structure 12, Structure 15, Structure 16, and Structure 17 of Table A.

In certain aspects, the rhNaGlu carries at most 1, 2, 3, 4, or 5 fucose residues. In more specific aspects, such a protein carries no fucose residue. In certain aspects, a population of such proteins comprises substantially no fucose residues (i.e., less than 2% of proteins contain fucose, less than 1%, less than 0.8%, less than 0.5%, less than 0.2%, or less than 0.1% of proteins in the population of proteins carry fucose). In certain aspects, the glycans in a population of such proteins contain substantially no fucose residues (i.e., less than 2% of the glycans contain fucose, less than 1%, less than 0.8%, less than 0.5%, less than 0.2%, or less than 0.1% of the glycans in a population of such proteins carry fucose).

In certain aspects, the rhNaGlu carries at most 1, 2, 3, 4, or 5 galactose residues. In more specific aspects, such a protein carries no galactose residue. In certain aspects, a population of such proteins comprises substantially no galactose residues (i.e., less than 2% of proteins contain galactose, less than 1%, less than 0.8%, less than 0.5%, less than 0.2%, or less than 0.1% of proteins in the population of proteins carry galactose). In certain aspects, the glycans in a population of such proteins contain substantially no galactose residues (i.e., less than 2% of the glycans contain galactose, less than 1%, less than 0.8%, less than 0.5%, less than 0.2%, or less than 0.1% of the glycans in a population of such proteins carry galactose).

In certain aspects, the rhNaGlu carries at most 1, 2, 3, 4, or 5 terminal galactose residues. In more specific aspects, such a protein carries no terminal galactose residue. In certain aspects, a population of such proteins comprises substantially no terminal galactose residues (i.e., less than 2% of proteins contain terminal galactose, less than 1%, less than 0.8%, less than 0.5%, less than 0.2%, or less than 0.1% of proteins in the population of proteins carry terminal galactose). In certain aspects, the glycans in a population of such proteins contain substantially no terminal galactose residues (i.e., less than 2% of the glycans contain terminal galactose, less than 1%, less than 0.8%, less than 0.5%, less than 0.2%, or less than 0.1% of the glycans in a population of such proteins carry terminal galactose).

In some aspects, the rhNaGlu lacks sialic acid (i.e., lacks neuraminic acid; more specifically lacks N-acetylneuraminic acid). In certain aspects, the rhNaGlu comprises mannose-6-phosphate but is not sialylated (i.e., lacks neuraminic acid; more specifically lacks N-acetylneuraminic acid). More specifically, the rhNaGlu comprises at least 1, 2, 3, 4, 5, 6, 7, 8, 9, or at least 10 mannose-6-phosphate residues and at most 1, 2, 3, 4, 5, 6, 7, 8, 9, or at most 10 sialic acid residues. A population of such proteins comprises on average at least 1, 2, 3, 4, 5, 6, 7, 8, 9, or at least 10 mannose-6-phosphate residues per protein molecule and at most 1, 2, 3, 4, 5, 6, 7, 8, 9, or at most 10 sialic acid residues. In specific aspects, a population of such proteins comprises on average between 2 and 4, 2 and 3, or between 3 and 4 mannose-6-phosphate residues and no sialic acid or at most 1 sialic acid residue.

In certain aspects, the rhNaGlu comprises terminal mannose residues but is not sialylated (i.e., lacks neuraminic acid; more specifically lacks N-acetylneuraminic acid). More specifically, the rhNaGlu comprises at least 1, 2, 3, 4, 5, 6, 7, 8, 9, or at least 10 terminal mannose residues and at most 1, 2, 3, 4, 5, 6, 7, 8, 9, or at most 10 sialic acid residues. A population of such proteins comprises on average at least 1, 2, 3, 4, 5, 6, 7, 8, 9, or at least 10 terminal mannose residues per protein molecule and at most 1, 2, 3, 4, 5, 6, 7, 8, 9, or at most 10 sialic acid residues. In certain embodiments, the rhNaGlucomprises at least 1, 2, 3, 4, 5, 6, 7, 8, 9, or at least 10 N-glycans that are high mannose (such as bi-, tri, or tetrantennary high mannose glycans). In yet another embodiment, in a population of such proteins at least 5%, 10%, 20%, 30%, 40%, 50%, 60%, 70%, 80%, 90%, 95%, 98%, or at least 99%, or 100% of the proteins comprise at least 1, 2, 3, 4, 5, 6, 7, 8, 9, or at least 10 N-glycans that is high mannose (such as biantennary, triantennary, or tetraantennary).

In certain aspects, the rhNaGlu is N-glycosylated and comprises a fragment of SEQ ID NO:1, e.g., a fragment of the sequence of amino acids 24 to 743 of SEQ ID NO:1, wherein the fragment is at least 5, 10, 15, 20, 25, 30, 35, 40, 45, 50, 75, or at least 100 amino acids long. In certain embodiments, the protein also comprises amino acid sequences other than amino acid sequences of SEQ ID NO:1.

In certain aspects, the rhNaGlu is N-glycosylated and comprises an amino acid sequence that is at least 50%, 60%, 70%, 80%, 90%, 95%, 98%, 99%, or 100% identical to a fragment of SEQ ID NO:1, e.g., a fragment of the sequence of amino acids 24 to 743 of SEQ ID NO:1, wherein the fragment is at least 5, 10, 15, 20, 25, 30, 35, 40, 45, 50, 75, or at least 100 amino acids long. In certain embodiments, the protein comprises also amino acid sequences other than amino acid sequences of SEQ ID NO:1.

In certain aspects, the rhNaGlu has a pI of between 5.5 and 7.5, or 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5. In certain aspects, the pI of the protein that is capable of accumulating in the CNS after administration outside the BBB (e.g., by intravenous or intramuscular administration) is 0.1, 0.2, 0.3, 0.4, 0.5, 0.6, 0.7, 0.8, 0.9, or 1 higher than the predicted pI for that protein based solely on amino acid sequence. In certain aspects, the pI of the protein that is capable of accumulating in the CNS after administration outside the BBB (e.g., by intravenous or intramuscular administration) is about the same as the predicted pI for that protein solely based on amino acid sequence.

In certain aspects, the rhNaGlu carries a post-translation modification that facilitates membrane localization, such as myristoylation (attachment of myristate), palmitoylation (attachment of palmitate), isoprenylation or prenylation (the addition of an isoprenoid group (e.g. farnesol and geranylgeraniol, i.e., farnesylation, geranylgeranylation), or glypiation (glycosylphosphatidylinositol (GPI) anchor formation via an amide bond to C-terminal tail).

In Table A, the residue to the left of the curly brackets can be at any potential position of the structure to the right of the curly brackets. P=phosphate. A legend for the symbols is provided below Table A.

### Annexin

Without wishing to be bound by any particular theory, it is believed that proteins containing bisecting GlcNAc oligosaccharides can bind annexin, which, together with mannose 6-phosphate, facilitates transport of the protein across the BBB. Avian-produced rhNaGlu effectively binds to a 32 kD protein present in non-human primate serum and 30 kD protein present in rat serum. The 32 kD protein in non-human primate serum was identified as annexin as determined by LC-MS. A complex containing annexin may include one or more annexins or other non-annexin proteins to form, e.g., a heterodimer or heterotetramer. In certain embodiments, annexin is annexin A1, A2, A3, A4, A5, A8, A9, A10 or A13. One aspect of the invention relates to rhNaGlu comprising mannose 6-phosphate (M6P or bis-M6P) and an annexin-binding moiety to facilitate transport across intact BBB. In another embodiment, the annexin-binding moiety is bisecting GlcNAc (e.g., structures 1, 2, 3, 4, 5, and 6 shown in Table A), negatively charged phospholipids (e.g., phosphatidylserine), peptide having affinity for annexin (e.g., antibody or fragment thereof, or another annexin protein of the same or another isoform), negatively charged RNA which has binding affinity for annexin.

The present invention also contemplates compositions of modified rhNaGlu proteins produced from modified nucleic sequences of rhNaGlu. The modified nucleic acid sequences include deletions, insertions, or substitutions of different nucleotides resulting in a polynucleotide that encodes a functionally equivalent polynucleotide or polypeptide. The encoded protein may also contain deletions, insertions, or substitutions of amino acid residues that produce a silent change and result in a functionally equivalent protein or polypeptide. Deliberate amino acid substitutions can be made on the basis of similarity in polarity, charge, solubility, hydrophobicity, hydrophilicity, and/or the amphipathic nature of the residues as long as the biological activity of the NaGlu is retained. For example, negatively charged amino acids can include aspartic acid and glutamic acid; positively charged amino acids can include lysine and arginine; and amino acids with uncharged polar head groups having similar hydrophilicity values can include leucine, isoleucine, and valine; glycine and alanine; asparagine and glutamine; serine and threonine; phenylalanine and tyrosine.

In other aspects, the rhNaGlu can be modified such that it contains an additional moiety or second peptide. In one embodiment, transferrin receptor ligand (TfRL) can be attached to human NaGlu at N- or C-terminus of NaGlu protein. . In one embodiment, the transferrin receptor ligand can be attached to C-terminus of the NaGlu protein. In another embodiment, human NaGlu is fused to insulin-like growth factor receptor (IGF2R) ligand at N- or C-terminus of the NaGlu protein. In yet another embodiment, the NaGlu protein is fused to low density lipoprotein (LDL) receptor ligand at N- or C-terminus of the NaGlu protein. In one embodiment, the NaGlu protein is fused to a stretch of five to ten consecutive acidic amino acid residues. The acidic amino acid residues can include aspartic acid (D) or glutamic acid (E).

In one embodiment, the rhNaGlu is produced in a transgenic avian that contains a transgene encoding the NaGlu protein. In one embodiment, the rhNaGlu is produced in an oviduct cell (e.g., a tubular gland cell) of a transgenic avian (e.g., chicken (Gallus)). In one embodiment, the rhNaGlu is glycosylated in the oviduct cell (e.g., tubular gland cell) of the transgenic avian. In one embodiment, the rhNaGlu has a glycosylation pattern resulting from the rhNaGlu being produced in an oviduct cell of a transgenic avian. In one embodiment, the rhNaGlu can be isolated and purified from the content of the hard shell eggs laid by the transgenic avian. In one embodiment, the rhNaGlu can be isolated and purified from egg white of the transgenic avian.

The present invention also includes compositions of an isolated mixture of NaGlu proteins, such as a mixture of one or more fragments and full-length rhNaGlu (e.g., 24-743 set forth in SEQ ID NO:1). In one embodiment, a substantial portion of the mixture contains phosphorylated M6P. In one embodiment, at least 10%, 20%, 30%, 40%, 50%, 60%, 70%, 80%, 85%, 90% 95%, 97%, 98% or 99% of the rhNaGlu in the mixture contains M6P. In yet another embodiment, at least 50% of the isolated rhNaGlu in the mixture contains M6P. In yet another embodiment, at least 60% of the isolated rhNaGlu in the mixture contains M6P. In yet another embodiment, at least 70% of the isolated rhNaGlu in the mixture contains M6P. In yet another embodiment, at least 80% of the isolated rhNaGlu in the mixture contains M6P. In yet another embodiment, at least 90% of the isolated rhNaGlu in the mixture contains M6P. In yet another embodiment, at least 95% of the isolated rhNaGlu in the mixture contains M6P. In yet another embodiment, at least 96% of the isolated rhNaGlu in the mixture contains M6P. In yet another embodiment, at least 97% of the isolated rhNaGlu in the mixture contains M6P. In yet another embodiment, at least 98% of the isolated rhNaGlu in the mixture contains M6P. In yet another embodiment, at least 99% of the isolated rhNaGlu in the mixture contains M6P.

Optionally, the rhNaGlu protein produced from an avian or mammalian expression system (e.g., CHO, HEK293, or human skin fibroblast cell-line) can be further modified to achieve a favorable glycosylation pattern (i.e., an increased amount of M6P) for cellular uptake while retaining the biological activity. Additional terminal M6P can be introduced to the rhNaGlu by the general methods applied to other hydrolases as described in U.S. Pat. No. 6,679,165, U.S. Pat. No. 7,138,262, or U.S. Publication No. 2009/0022702, the entire teachings of each of which are incorporated herein by reference. For example, a highly phosphorylated mannopyranosyl oligosaccharide compound can be derivatized with a chemical compound containing a carbonyl-reactive group, followed by oxidizing the rhNaGlu protein to generate carbonyl (aldehyde) group on one glycan structure of the protein, and reacting the oxidized NaGlu protein with the glycan with the derivatized highly phosphorylated mannopyranosyl oligosaccharide compound to form a new compound having hydrazine bond.

### III. Vectors

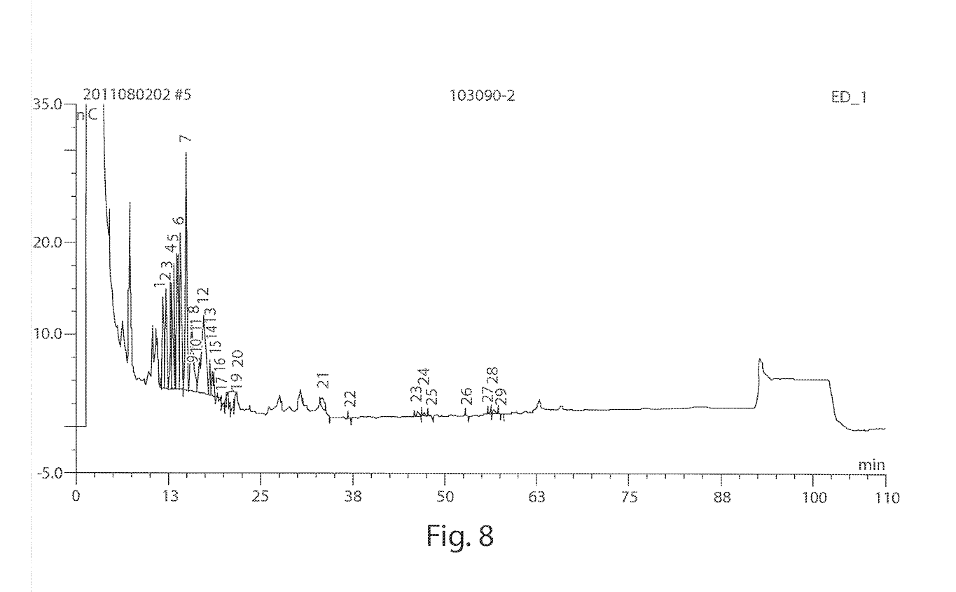

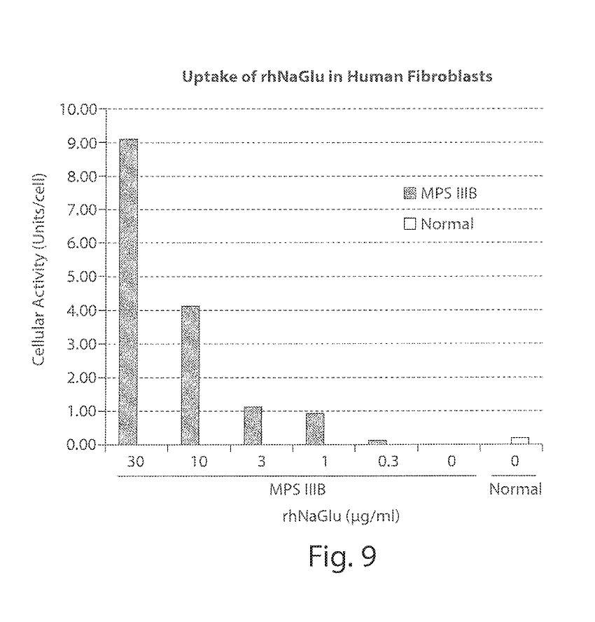

Methods which are well-known to those skilled in the art can be used to construct expression vectors containing sequences encoding NaGlu and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y., the entire teachings of which are incorporated herein by reference. A variety of expression vector/host systems can be utilized to express nucleic acid sequences encoding rhNaGlu. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus) or with bacterial expression vectors (e.g., Ti or pBR322 plasmids); or mammalian cell culture systems (e.g., pTT22 vector). Non-limiting examples of the pTT22 vector containing human NaGlu cDNA fused to a nucleic acid sequence encoding acidic amino acid residue and TfRL are shown in FIGS. 11 and 12.

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by vertebrate (e.g., avian or mammalian) cells have a signal peptide or secretory leader sequence which is cleaved from the mature protein once export of the growing protein chain across the rough endoplasmic reticulum (ER) has been initiated. Those of ordinary skill in the art are aware that polypeptides produced in the ER by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., the MEAVAVAAAVGVLLLAGAGGAAG (1-23 of SEQ ID NO:1) signal peptide of human NaGlu is used, or a functional derivative of that sequence that retains the ability to direct the secretion of the polypeptide that is operably associated with it. Alternatively, a heterologous signal peptide (e.g., a heterologous mammalian or avian signal peptide), or a functional derivative thereof, may be used. For example, the wild-type leader sequence may be substituted with the leader sequence of, for example, human tissue plasminogen activator (tPA) or mouse B-glucuronidase.

The control elements or regulatory sequences can includes those non-translated regions of the vector-enhancers, promoters, 5′ and 3′ untranslated regions that interact with host cellular proteins to carry out transcription and translation. Such elements can vary in their strength and specificity. Depending on the vector system and host cell utilized, any number of suitable transcription and translation elements can be used. For example, when cloning in bacterial systems, inducible promoters such as the hybrid lac-Z promoter of the Bluescript™ phagemid (Stratagene, LaJolla, California) or pSport1™ plasmid (Gibco BRL) and the like can be used. In mammalian cell systems, promoters from mammalian genes or from mammalian viruses are preferred. If it is necessary to generate a cell line that contains multiple copies of the sequence encoding NaGlu, vectors based on SV40 or EBV can be also used with an appropriate selectable marker such as puromycin and ampicillin (see, e.g., FIGS. 11 and 12).

When the rhNaGlu is produced in a transgenic avian, the present invention contemplates that the rhNaGlu sequence be placed downstream of a promoter such that the sequence encoding the rhNaGlu can be expressed in a tissue-specific manner in a transgenic avian. For example, the promoter can be an oviduct-specific promoter that is largely, but not entirely, specific to the magnum, such as the oviduct-specific promoter, including but not limited to, ovalbumin, lysozyme, conalbumin, ovomucoid, ovomucoid, ovomucin and ovotransferrin promoters. In one embodiment, the promoter is an ovalbumin promoter, a lysozyme promoter, a conalbumin promoter, an ovomucoid promoter, an ovomucin promoter and/or an ovotransferrin promoter or any functional portion thereof.

Alternatively, a constitutive promoter can be used to express the coding sequence of human NaGlu in an avian. In this case, expression is not limited to the magnum; expression also occurs in other tissues within the avian (e.g., blood). The use of such a transgene, which includes a constitutive promoter and the coding sequence of NaGlu, is also suitable for effecting or driving the expression of a protein in the oviduct and the subsequent secretion of the protein into the egg. In one embodiment, the constitutive promoter can be, for example, a cytomegalovirus (CMV) promoter, a rous-sarcoma virus (RSV) promoter, a murine leukemia virus (MLV) promoter, and β-actin promoter. In one embodiment, the promoter is a CMV promoter, a MDOT promoter, a RSV promoter, a MLV promoter, or a mouse mammary tumor virus (MMTV) promoter of any functional portion thereof.

The invention also contemplates any useful fragment or component of the promoters described herein. The promoter can be at least one segment, fragment or component of a promoter region, such as a segment of the ovalbumin, lysozyme, conalbumin, ovomucoid, ovomucin, ovotransferrin, CMV, RSV or MLV promoter region. In a preferred embodiment, the promoter is a segment of the oviduct-specific promoter region which contains essential elements to direct expression of the coding sequence in the tubular gland cells. For example, included in the scope of the present invention is a segment, portion or fragment of an oviduct-specific promoter and/or condensing the critical regulatory elements of the oviduct-specific promoter so that it retains sequences required for expression in the tubular gland cells of the magnum of the oviduct. In one embodiment, a segment of the ovalbumin promoter region is used. This segment comprises the 5′-flanking region of the ovalbumin gene.

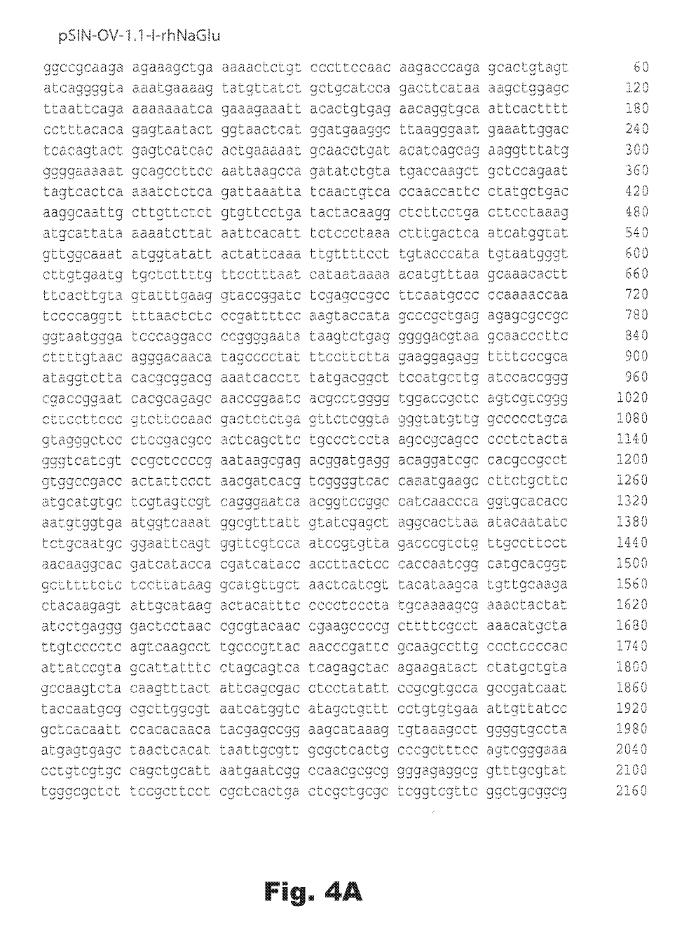

A vector that contains a coding sequence for human NaGlu can be used for transfecting blastodermal cells of an avian or mammalian cell to generate stable integrations into the avian or mammalian genome and to create a germline transgenic avian or mammalian cell line. A non-limiting example of such vector is shown in FIGS. 4A-D and 5. In the avian expression system, the human NaGlu coding sequence is operably linked to a promoter in a positional relationship to express the coding sequence in a transgenic avian, particularly in the tubular gland cell of the magnum of the avian oviduct, such that the recombinant human NaGlu protein is expressed and deposited in egg white of a hard shell egg laid by the transgenic avian. Additional suitable vectors and methods to making vectors for expressing rhNaGlu in an avian system are also disclosed in U.S. Pat. No. 6,730,822; U.S. Pat. No. 6,825,396; U.S. Pat. No. 6,875,588; U.S. Pat. No. 7,294,507; U.S. Pat. No. 7,521,591; U.S. Pat. No. 7,534,929; U.S. Publication No. 2008/0064862A1; and U.S. Patent Publication No. 2006/0185024, the entire teachings of which are incorporated herein by reference. Non-limiting examples of other promoters which can be also useful in the present invention include Pol III promoters (for example, type 1, type 2 and type 3 Pol III promoters) such as H1 promoters, U6 promoters, tRNA promoters, RNase MPR promoters and functional portions of each of these promoters. Typically, functional terminator sequences are selected for use in the present invention in accordance with the promoter that is employed.

In one embodiment, the vector is a retroviral vector, in which the coding sequence and the promoter are both positioned between the 5′ and 3′ LTRs of the retroviral vector. In one useful embodiment, the LTRs or retroviral vector is derived from an avian leukosis virus (ALV), a murine leukemia virus (MLV) or a lentivirus. One useful retrovirus for randomly introducing a transgene into the avian genome is the replication-deficient ALV, the replication-deficient MLV, or the replication-deficient lentivirus.

The present invention also contemplates the use of self-inactivating (SIN) vectors. SIN vectors can be useful for increasing the quantity of human NaGlu produced in the oviduct of a transgenic avian. This effect can be further enhanced when the SIN vector does not contain any selectable marker cassette with a functional promoter (SIN/SC negative vector). In one embodiment, a SIN vector is a retroviral vector having altered genome so that the 5′ LTR of the integrated retroviral vector does not function as a promoter. In one particular embodiment, a portion or all of the nucleotide sequence of the retroviral vector that results in the U3 region of the 5′ LTR of the retroviral vector once integrated can be deleted or altered in order to reduce or eliminate promoter activity of the 5′ LTR. A non-limiting example of SIN vector which contains an ovalbumin promoter region fused to the coding sequence of human rhNaGlu is shown in FIGS. 4A-D and 5. Functional components of the vector are also tabulated in Table 1.

TABLE 1
Functional components in pSIN-OV-1.1 kb-I-rhNaGlu

| Functional components | Nucleotide Sequence in SEQ ID NO: 4 |
|---|---|
| poly A site | 634-639 |
| Partial gag | 692-945 |
| LTR (RAV2) | 1243-1588 |
| Partial LTR (RAV2) | 4691-4863 |
| ALV CTE | 4899-4986 |
| 1.1 kb Ovalbumin promoter | 5232-6363 |
| DHS II | 5334-5714 |
| DHS I | 6064-6364 |
| Exon L | 6364-6410 |
| Intron 1 | 6411-7999 |
| NaGlu | 8017-10248 |

Any of the vectors described herein can include a sequence encoding a signal peptide that directs secretion of the protein expressed by the vector's coding sequence from, for example, the tubular gland cells of the oviduct of an avian. Where a recombinant human NaGlu protein would not otherwise be secreted, the vector containing the coding sequence is modified to comprise a DNA sequence comprising about 60 bp encoding a signal peptide from, for example, the lysozyme gene. The DNA sequence encoding the signal peptide is inserted in the vector such that it is located at the N-terminus of the rhNaGlu protein encoded by the DNA.

Further, the coding sequences of vectors used in any of the methods of the present invention can be provided with a 3′ untranslated region (3′ UTR) to confer stability to the RNA produced. When a 3′ UTR is added to a retroviral vector, the orientation of the promoter, the coding sequence and the 3′ UTR is preferably reversed with respect to the direction of the 3′ UTR, so that the addition of the 3′ UTR does not interfere with transcription of the full-length genomic RNA. In one embodiment, the 3′ UTR may be that of the ovalbumin gene, lysozyme gene or any 3′ UTR that is functional in a magnum cell, i.e., the SV40 late region.

### IV. Transgenic Avians

Transgenes described herein can be introduced into avian embryonic blastodermal cells to produce a transgenic chicken, transgenic turkey, transgenic quail and other avian species that carry the transgene encoding recombinant human NaGlu in the genome of its germ-line tissue. In one aspect of the invention, a transgenic avian that produces rhNaGlu is created by transduction of embryonic blastodermal cells with replication-defective or replication-competent retroviral particles carrying the transgene between the 5′ and 3′ LTRs of the retroviral vector. For instance, an avian leukosis virus (ALV) retroviral vector or a murine leukemia virus (MLV) retroviral vector can be used. An RNA copy of the modified retroviral vector packaged into viral particles can be used to infect embryonic blastoderms which develop into transgenic avians.

By the methods of the present invention, transgenes can be introduced into embryonic blastodermal cells of various avian species. For example, the methods can be applied to produce a transgenic chicken, transgenic turkey, transgenic quail, transgenic duct, and other avian species, that carry the transgene in the genome of its germ-line tissue in order to produce proteins of the invention. The blastodermal cells are typically stage VII-XII cells as defined by Eyal-Giladi and Kochav (1976), or the equivalent thereof. In a preferred embodiment, the blastoderm cells are at or near stage X.

In one method of transfecting blastodermal cells, a packaged retroviral-based vector can be used to deliver the vector into embryonic blastodermal cells so that the vector is integrated into the avian genome. Such viral particles (i.e., transduction particles) are produced for the vector and titered to determine the appropriate concentration that can be used to inject embryos. In one embodiment, avian eggs are windowed according to the procedure described in U.S. Pat. No. 5,897,998, the disclosure of which is incorporated herein by reference in its entirety, and the eggs are injected with transducing particles at or near stage X.

The transgenic avians of the invention which produce rhNaGlu are developed from the blastodermal cells into which the vector has been introduced. The resulting embryo is allowed to develop and the chick allowed to mature. At this stage, the transgenic avian produced from blastodermal cells is known as a founder and is chimeric with respect to the cells carrying the transgene and is referred to G0. G0 founder avians are typically chimeric for each inserted transgene. That is, only some of the cells of the G0 transgenic bird contain the transgene. Some founders carry the transgene in tubular gland cells in the magnum of their oviducts. These avians express the rhNaGlu protein encoded by the transgene in their oviducts. The NaGlu protein may also be expressed in other tissues (e.g., blood) in addition to the oviduct. Some founders are germ-line founders that carry the transgene in the genome of the germ-line tissues, and may also carry the transgene in oviduct magnum tubular gland cells that express the exogenous protein.

The transgenic avian can carry the transgene in its germ-line providing transmission of the exogenous transgene to the avian's offspring stably in a Mendelian fashion. The G0 generation is typically hemizygous for the transgene encoding rhNaGlu. The G0 generation can be bred to non-transgenic animals to give rise to G1 transgenic offspring which are also hemizygous for the transgene and contain the transgene in essentially all of the bird's cells. The G1 hemizygous offspring can be bred to non-transgenic animals giving rise to G2 hemizygous offspring or may be bred together to give rise to G2 offspring homozygous for the transgene. Substantially all of the cells of avians which are positive for the transgene that are derived from G1 offspring contain the transgene. In one embodiment, hemizygotic G2 offspring from the same line can be bred to produce G3 offspring homozygous for the transgene. In another embodiment, hemizygous G0 or G1 animals, for example, are bred together to give rise to homozygous G1 offspring containing two copies of the transgene(s) in each cell of the animal. These are merely examples of certain useful breeding methods and the present invention contemplates the employment of any useful breeding method such as those known to individuals of ordinary skill in the art.

V. Production of rhNaGlu

The rhNaGlu can be produced using a transgenic avian that contains in the genome a transgene encoding rhNaGlu. In one embodiment, the transgenic avian is a germline transgenic chicken, quail, duck or turkey. In one particularly useful embodiment, the invention is drawn to the production of NaGlu which can be produced in the oviduct of a chicken.

Production of rhNaGlu with or without modification in the avian system (e.g., in the avian oviduct) is within the scope of the invention. In one embodiment, the unmodified rhNaGlu comprises the wild-type amino acid sequence (24-743 of SEQ ID NO:1) with a glycosylation structure (i.e., M6P or bis-M6P) that enables efficient uptake by human cells. In another embodiment, the modified protein can be an rhNaGlu fusion protein having a glycosylation pattern (i.e., M6P or bis-M6P) that enables efficient uptake by human cells.

A suitable avian vector that contains a nucleic acid sequence encoding a NaGlu protein, operably linked to a tissue-specific or constitutive promoter that drives expression of the encoding sequence in the chicken oviduct are introduced into chicken embryonic cells at or near stage X as described herein. The transformed embryonic cells are incubated under conditions conducive to hatching live chicks. Live chicks are nurtured into a mature chimeric chicken which are mated with a non-transgenic chicken naturally or via artificial insemination. A transgenic chicken is identified by screening progeny for germline incorporation of the protein encoding sequence. The transgenic progeny can be mated with another transgenic or a non-transgenic chicken to produce a fully germline transgenic hen that lays eggs.

The rhNaGlu can be produced in a tissue-specific manner. For example, rhNaGlu can be expressed in the oviduct, blood and/or other cells or tissues of the transgenic avian. In one embodiment, the NaGlu is expressed in the tubular gland cells of the magnum of the oviduct of the transgenic avian, secreted into the lumen of the oviduct, and deposited into egg white. In one embodiment, egg white containing rhNaGlu is harvested and stored in bulk at a temperature ranging from 4° C. to −20° C. The NaGlu is then isolated and purified from the contents of the eggs using various methods known in the art.

One aspect of the present invention relates to avian hard shell eggs (e.g., chicken hard shell eggs) which contain the rhNaGlu protein. The rhNaGlu produced and secreted by the transgenic avian is glycosylated in a manner favorable to cellular uptake by a human cell. The protein may be present in any useful amount. In one embodiment, the protein is present in an amount in a range between about 0.01 μg per hard-shell egg and about 1 gram per hard-shell egg. In another embodiment, the protein is present in an amount in a range of between about 1 μg per hard-shell egg and about 1 gram per hard-shell egg. For example, the protein may be present in an amount in a range of between about 10 μg per hard-shell egg and about 1 gram per hard-shell egg (e.g., a range of between about 10 μg per hard-shell egg and about 400 milligrams per hard-shell egg).

In one embodiment, the rhNaGlu is present in the egg white of the egg. In one embodiment, the rhNaGlu is present in an amount in a range of between about 1 ng per milliliter of egg white and about 0.2 gram per milliliter of egg white. For example, the rhNaGlu may be present in an amount in a range of between about 0.1 μg per milliliter of egg white and about 0.2 gram per milliliter of egg white (e.g., the rhNaGlu may be present in an amount in a range of between about 1 μg per milliliter of egg white and about 100 milligrams per milliliter of egg white. In one embodiment, the rhNaGlu is present in an amount in a range of between about 1 μg per milliliter of egg white and about 50 milligrams per milliliter of egg white. For example, the rhNaGlu may be present in an amount in a range of about 1 μg per milliliter of egg white and about 10 milligrams per milliliter of egg white (e.g., the rhNaGlu may be present in an amount in a range of between about 1 μg per milliliter of egg white and about 1 milligrams per milliliter of egg white). In one embodiment, the rhNaGlu is present in an amount of more than 0.1 μg per milliliter of egg white. In one embodiment, the rhNaGlu is present in an amount of more than 0.5 μg per milliliter of egg white. In one embodiment, the rhNaGlu is present in an amount of more than 1 μg per milliliter of egg white. In one embodiment, the protein is present in an amount of more than 1.5 μg per milliliter of egg white. In one embodiment, the rhNaGlu is present in an amount of more than 0.5 μg per milliliter of egg white. In one embodiment, the protein is present in an amount of more than 0.1 μg per milliliter of egg white.

In one embodiment, the rhNaGlu is present in an amount of 20 mg/L, 30mg/L, 40mg/L, 50 mg/L, 60 mg/L, 70 mg/L, 80 mg/L, 90 mg/L, 100 mg/L, 120 mg/L, 130 mg/L, 140 mg/L, 150 mg/L, 160 mg/L, 170 mg/L, 200 mg/L, 300mg/L, 400 mg/L, 500 mg/L, 600 mg/L, 700 mg/L, 800 mg/L, 900 mg/L, or 1,000 mg/L egg white. In one embodiment, the rhNaGlu is present in an amount of about 100 mg/L of egg white. In one embodiment, the rhNaGlu is present in an amount of about 200 mg/L of egg white.

### VI. Host Cells

The present invention also contemplates rhNaGlu produced in any useful protein expression system including, without limitation, cell culture (e.g., avian cells, CHO cells, HEK293 cells and COS cells), yeast, bacteria, and plants.

A host cell strain can be chosen for its ability to modulate the expression of the inserted sequences or to process the expressed NaGlu in the desired fashion. Such modifications of the polypeptide of NaGlu include, without limitation, glycosylation, phosphorylation, or lipidation. Different host cells such as CHO, COS, HeLa, MDCK, HEK293 and W138, which have specific cellular machinery and characteristic mechanisms for such post-translational activities, can be chosen to ensure the correct modification and processing of the fusion protein of the present invention. An avian tumor cell line is also contemplated as a host cell for expressing the polypeptide of the present invention. Examples of a useful avian cell line (e.g., an avian oviduct tumor cell line) are described in U.S. Pat. Publication No. 2009/0253176, the entire teachings of which are incorporated herein by reference.

### VII. Pharmaceutical Compositions

The present invention also features pharmaceutical compositions comprising isolated and substantially purified rhNaGlu or a pharmaceutically acceptable salt thereof. The recombinant human NaGlu proteins may be administered using one or more carriers, e.g., as part of a pharmaceutical formulation, or without a carrier. The carrier(s) must be “acceptable” in the sense of being compatible with the other ingredients of the formulation and not deleterious to the recipient thereof. Compositions comprising such carriers, including composite molecules, are formulated by well-known conventional methods (see, for example, Remington's Pharmaceutical Sciences, 14th Ed., Mack Publishing Co., Easton, Pa.), the entire teachings of which are incorporated herein by reference. The carrier may comprise a diluent. In one embodiment, the pharmaceutical carrier can be a liquid and the protein may be in the form of a solution. The pharmaceutical carrier can be wax, fat, or alcohol. In another embodiment, the pharmaceutically acceptable carrier may be a solid in the form of a powder, a lyophilized powder, or a tablet. In one embodiment, the carrier may comprise a liposome or a microcapsule.

In some embodiments, a pharmaceutical composition comprising recombinant human NaGlu protein further comprises a buffer. Exemplary buffers include acetate, phosphate, citrate and glutamate buffers. Exemplary buffers also include lithium citrate, sodium citrate, potassium citrate, calcium citrate, lithium lactate, sodium lactate, potassium lactate, calcium lactate, lithium phosphate, sodium phosphate, potassium phosphate, calcium phosphate, lithium maleate, sodium maleate, potassium maleate, calcium maleate, lithium tartarate, sodium tartarate, potassium tartarate, calcium tartarate, lithium succinate, sodium succinate, potassium succinate, calcium succinate, lithium acetate, sodium acetate, potassium acetate, calcium acetate, and mixtures thereof. In some embodiments, the buffer is trisodium citrate dihydrate. In some embodiments, the buffer is citric acid monohydrate. In some embodiments, a pharmaceutical composition comprises trisodium citrate dihydrate and citric acid monohydrate.

In some embodiments, a pharmaceutical composition comprising recombinant human NaGlu protein further comprises a stabilizer. Exemplary stabilizers include albumin, trehalose, sugars, amino acids, polyols, cyclodextrins, salts such as sodium chloride, magnesium chloride, and calcium chloride, lyoprotectants, and mixtures thereof. In some embodiments, a pharmaceutical composition comprises human serum albumin.

In some embodiments, it is desirable to add a surfactant to the pharmaceutical composition. Exemplary surfactants include nonionic surfactants such as Polysorbates (e.g., Polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68, etc). Typically, the amount of surfactant added is such that it reduces aggregation of the protein and minimizes the formation of particulates or effervescences. For example, a surfactant may be present in a formulation at a concentration from about 0.001-0.5% (e.g., about 0.005-0.05%, or 0.005-0.01%). In particular, a surfactant may be present in a formulation at a concentration of approximately 0.005%, 0.01%, 0.02%, 0.1%, 0.2%, 0.3%, 0.4%, or 0.5%, etc. Ranges and values intermediate to the above recited ranges and values are also contemplated to be part of the invention.

In some embodiments, suitable pharmaceutical compositions of the invention may further include one or more bulking agents, in particular, for lyophilized formulations. A “bulking agent” is a compound which adds mass to the lyophilized mixture and contributes to the physical structure of the lyophilized cake. For example, a bulking agent may improve the appearance of lyophilized cake (e.g., essentially uniform lyophilized cake). Suitable bulking agents include, but are not limited to, sodium chloride, lactose, mannitol, glycine, sucrose, trehalose, hydroxyethyl starch. Exemplary concentrations of bulking agents are from about 1% to about 10% (e.g., 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10.0%). Ranges and values intermediate to the above recited ranges and values are also contemplated to be part of the invention. The pharmaceutical compositions can be in the form of a sterile lyophilized powder for injection upon reconstitution with a diluent. The diluent can be water for injection, bacteriostatic water for injection, or sterile saline. The lyophilized powder may be produced by freeze drying a solution of the fusion protein to produce the protein in dry form. As is known in the art, the lyophilized protein generally has increased stability and a longer shelf-life than a liquid solution of the protein.

Pharmaceutical formulations include those suitable for oral, rectal, nasal, topical (including buccal and sub-lingual), vaginal or parenteral administration. Preferably, the pharmaceutical formulations of the invention include those suitable for administration by injection including intrathecal, intraparenchymal, intracerebral, intraventricular, intramuscular, sub-cutaneous and intravenous administration. In one embodiment, the formulations of the invention are suitable for intravenous administration. In another embodiment, the formulations of the invention are suitable for intrathecal administration. The pharmaceutical formulations of the invention also include those suitable for administration by inhalation or insufflation. The formulations can, where appropriate, be conveniently presented in discrete dosage units and can be prepared by any of the methods well known in the art of pharmacy. The methods of producing the pharmaceutical formulations typically include the step of bringing the therapeutic proteins into association with liquid carriers or finely divided solid carriers or both and then, if necessary, shaping the product into the desired formulation.

Recombinant human NaGlu proteins of the invention can also be formulated for parenteral administration (e.g., by injection, for example bolus injection or continuous infusion) and can be presented in unit dose form in ampoules, pre-filled syringes, small volume infusion or in multi-dose containers with an added preservative. The therapeutic proteins can be injected by, for example, subcutaneous injections, intramuscular injections, intrathecal injections, intracerebral injections, intraparenchymal injections, intraventricular injections, and intravenous (IV) infusions or injections.

In one embodiment, the recombinant human NaGlu protein is administered intravenously by IV infusion by any useful method. In one example, the recombinant human NaGlu protein can be administered by intravenous infusion through a peripheral line. In another example, the recombinant human NaGlu protein can be administered by intravenous infusion through a peripherally inserted central catheter. In another example, the recombinant human NaGlu protein can be administered by intravenous infusion facilitated by an ambulatory infusion machine attached to a venous vascular access port. In one embodiment of intravenous infusion, the medication is administered over a period of 1 to 8 hours depending on the amount of medication to be infused and the patient's previous infusion-related reaction history, as determined by a physician skilled in the art. In another embodiment, the recombinant human NaGlu protein is administered intravenously by IV injection. In another embodiment, the recombinant human NaGlu protein can be administered via intraperitoneal or intrathecal injection.

In some embodiments, the therapeutic proteins are administered by infusion, and the infusion can occur over an extended time period, for example, 30 minutes to 10 hours. Thus, the infusion can occur, for example, over a period of about 1 hour, about 2 hours, about 3 hours, about 4 hours, or about 5 hours. The infusion can also occur at various rates. Thus, for example, the infusion rate can be about 1 mL per hour to about 20 mL per hour. In some embodiments, the infusion rate is 5 mL to 10 mL per hour. In one embodiment, the infusion rate is 1, 2, 3, 4, 5, 6, 7, 8, 9, 10, 11, 12, 13, 14, 15, 16, 17, 18, 19 or 20 mL per hour. In one embodiment, the infusion rate is 0.1 to 5 mg/kg/hr. In one embodiment, the infusion rate is about 0.1, about 0.2, about 0.3, about 0.5, about 1.0, about 1.5, about 2.0, or about 3 mg/kg/hr. Ranges and values intermediate to the above recited ranges and values are also contemplated to be part of the invention.

The therapeutic proteins can take such forms as suspensions, solutions, or emulsions in oily or aqueous vehicles, and can contain formulatory agents such as suspending, stabilizing and/or dispersing agents. The recombinant human NaGlu proteins can be in powder form, obtained by aseptic isolation of sterile solid or by lyophilization from solution, for constitution with a suitable vehicle, e.g., sterile, pyrogen-free water, before use.

Formulations in accordance with the present invention can be assessed based on product quality analysis, reconstitution time (if lyophilized), quality of reconstitution (if lyophilized), high molecular weight, moisture, and glass transition temperature. Typically, protein quality and product analysis include product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC (RP-HPLC), multi-angle light scattering (MALS), fluorescence, ultraviolet absorption, nephelometry, capillary electrophoresis (CE), SDS-PAGE, and combinations thereof. In some embodiments, evaluation of product in accordance with the present invention may include a step of evaluating appearance (either liquid or cake appearance).

Generally, formulations (lyophilized or aqueous) can be stored for extended periods of time at room temperature. Storage temperature may typically range from 0° C. to 45° C. (e.g., 4° C., 20° C., 25° C. or 45° C.). Formulations may be stored for a period of months to a period of years. Storage time generally will be 24 months, 12 months, 6 months, 4.5 months, 3 months, 2 months or 1 month. Formulations can be stored directly in the container used for administration, eliminating transfer steps. Ranges and values intermediate to the above recited ranges and values are also contemplated to be part of the invention.

Formulations can be stored directly in the lyophilization container (if lyophilized), which may also function as the reconstitution vessel, eliminating transfer steps. Alternatively, lyophilized product formulations may be measured into smaller increments for storage. Storage should generally avoid circumstances that lead to degradation of the proteins, including but not limited to exposure to sunlight, UV radiation, other forms of electromagnetic radiation, excessive heat or cold, rapid thermal shock, and mechanical shock. The pharmaceutical compositions according to the invention can also contain other active ingredients such as immunosuppressive agents, antimicrobial agents, or preservatives, discussed in more detail below.

### VIII. Kits

The present invention further provides kits or other articles of manufacture which contain the recombinant human NaGlu of the present invention and provide instructions for its reconstitution (if lyophilized) and/or use. Kits or other articles of manufacture may include a container, a catheter and any other articles, devices or equipment useful in intravenous or intrathecal administration and associated surgery. Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, a label on, or associated with, the container may indicate directions for use and/or reconstitution. For example, the label may indicate that the formulation is reconstituted to protein concentrations as described above. The label may further indicate that the formulation is useful or intended for, for example, intravenous or intrathecal administration. In some embodiments, a container may contain a single dose of a stable formulation containing a replacement enzyme (e.g., a recombinant NaGlu protein). In various embodiments, a single dose of the stable formulation is present in a volume of less than about 15 mL, 10 mL, 5.0 mL, 4.0 mL, 3.5 mL, 3.0 mL, 2.5 mL, 2.0 mL, 1.5 mL, 1.0 mL, or 0.5 mL. Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation will generally be at least 1 mg/mL (e.g., at least 5 mg/mL, at least 10 mg/mL, at least 25 mg/mL, at least 50 mg/ mL, at least 75 mg/mL, at least 100 mg/mL).

Kits or other articles of manufacture may further include other materials desirable from a commercial and user standpoint, including other buffers, diluents, filters, needles, catheters, syringes, and package inserts with instructions for use. Ranges and values intermediate to the above recited ranges and values are also contemplated to be part of the invention.

### EXAMPLES

The following specific examples are intended to illustrate the invention and should not be construed as limiting the scope of the claims. The contents of all figures and all references, patents and published patent applications cited throughout this application, as well as the Figures, are expressly incorporated herein by reference in their entirety.

### Example 1

Purification of rhNaGlu

rhNaGlu protein was purified by using methods known in the art. Egg white (EW) containing rhNaGlu was solubilized at pH 6 overnight and clarified through centrifugation and/or depth filtration. The EW was adjusted with 1 M NaOAc buffer (pH 4) to pH 6. For the depth filtration process, T2600 filter (Pall™, 40 um) was used as a 1^{st} filtration and then PDF 1 (Pall™, K200P,15 um +EKS, 0.22 um) as a 2^{nd} filtration step. The filters are single-use membrane with an optimized capacity 60 L EW/m² for each filter. The hold volume of membrane is 2 L/m² for T2600 and 4-5 L/m² for PDF 1. In the process, the hold volume was discarded before the filtered

EW collected. The buffer (20 mM Phosphate/137 mM NaCl, pH 6) equivalent to the membrane hold volume was used to chase EW left on the filters.

A phenyl-HIC (hydrophobic interaction chromatography) column was applied as a capture step. Since most of egg white proteins are hydrophilic, 99% of egg white proteins passed through the HIC column into flow through. rhNaGlu has a higher hydrophobicity binding to phenyl-HIC. Levels of rhNaGlu deposited in egg white of a transgenic avian were 0.1-0.2 g/L.

Egg white containing rhNaGlu was loaded onto the column with a ratio of 30:1. After completion of loading, the column was washed with the equilibration buffer, 5 mM phosphate buffer, pH 6, and 5 mM Tris buffer, pH 7.2. rhNaGlu was eluted with 30% propylene glycol, pH 7.2. After the completion of loading, the column was washed with equilibration buffer and 5 mM phosphate buffer (pH 6). rhNaGlu was eluted with 30% propylene glycol with 5 mM Tris buffer (pH 7.2). The column binding capacity is approximately 4.5 mg/mL. The purity of rhNaGlu through the phenyl-HIC column can be reached to >95% (950 time increase). The recovery is approximately 80% with 30% of propylene glycol elution.

The eluted rhNaGlu fraction was adjusted to pH 5 with 1 M acetic acid and then loaded onto a GigaCap S column (EW: column size=10:1). The column was equilibrated with 50 mM NaOAc buffer (pH 5). After completion of loading, the column was washed with the equilibration buffer. The rhNaGlu was eluted with 50 mM NaOAc/60 mM NaCl (pH 5).

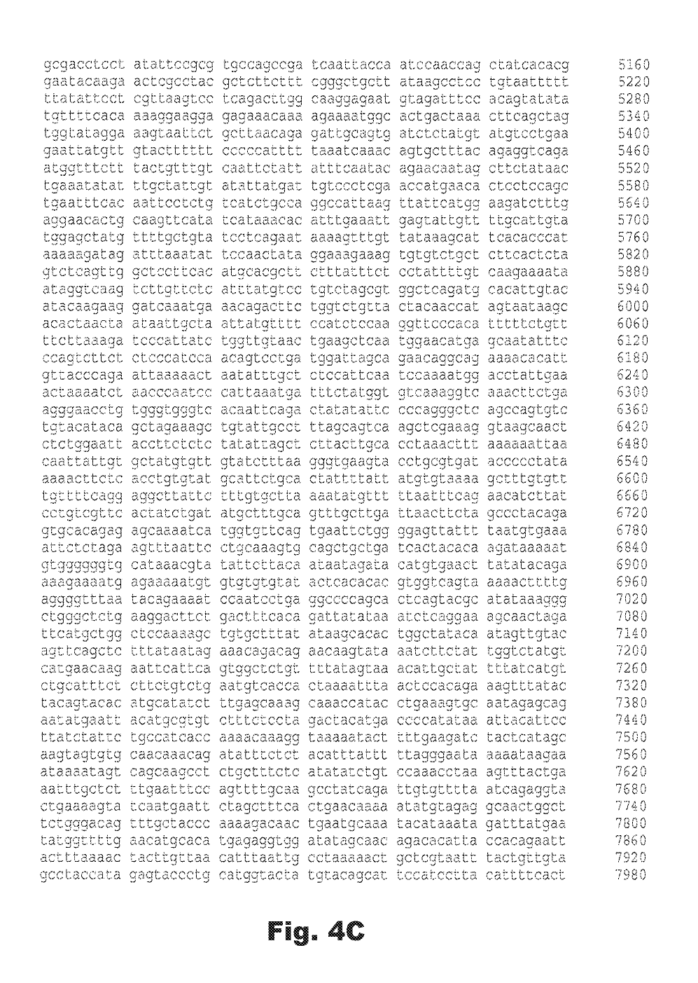

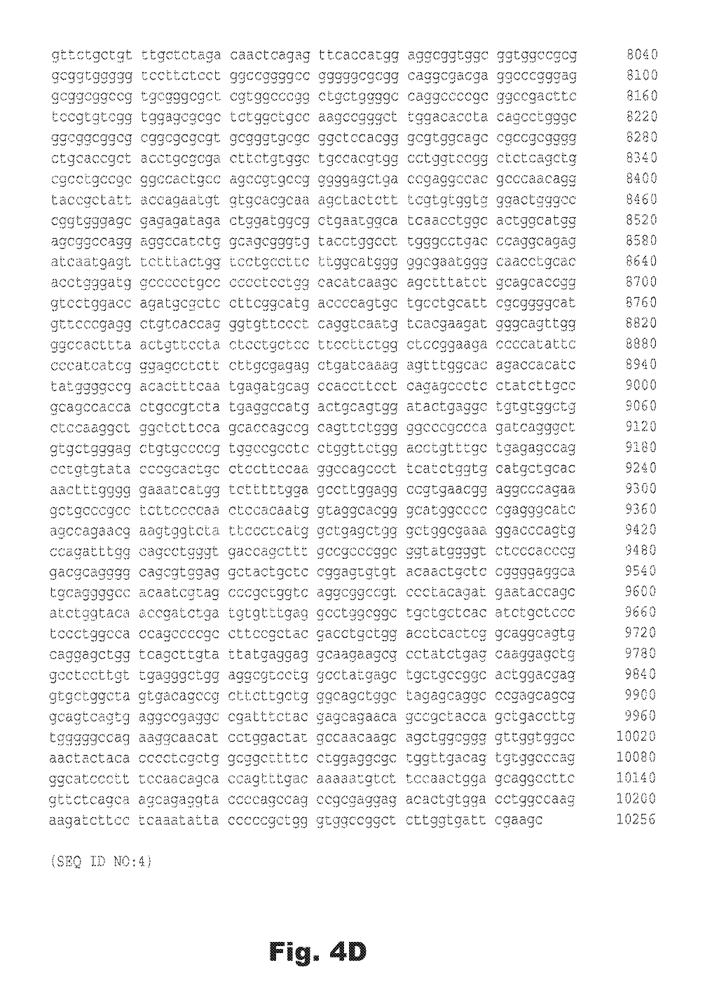

The protein characterization was performed using purified rhNaGlu. The molecular weight of rhNaGlu (˜90 kDa) purified from egg white was analyzed on SDS-PAGE (FIG. 6). The average expression level of rhNaGlu in egg white is shown in FIG. 7. The characteristics of rhNaGlu produced from the transgenic avian are summarized in Table 2.

TABLE 2

| | rhNaGlu (Gallus) |
|---|---|
| Apparent Molecular Weight | ~90 kDa |
| pI | 6.1-6.9 |
| pH Stability | pH 5-8 |
| Stability in Egg White | >50 days |
| Expression Level | 0.1-0.2 g/L |

### Example 2

Stability of rhNaGlu in Egg White

A single egg was cracked 7 days post-lay and analyzed for activity. Contents were divided in half and each half was subject to standard egg white clarification. Both untreated and clarified egg whites were aliquoted and stored at 4° C. and −20° C. for enzyme activity stability. It was demonstrated that rhNaGlu in egg white showed stable enzyme activity at least up to 50 days.

Freeze/thaw cycle stability was assessed. The purified rhNaGlu was frozen in liquid nitrogen for 10 seconds and thawed at 37° C. for 2 min. The enzyme activity showed no change for 10 cycles.

The purified rhNaGlu was dialyzed into different pH buffers to measure the stability of pure enzyme. The results showed that pure rhNaGlu was stable between pH 5-8 within 12 days.

### Example 3

### Oligosaccharide Profiling

Mannose-6-phosphate (M6P) is a terminal monosaccharide of N-linked oligosaccharides that is an important part of the tertiary structure of glycoprotein and, when incorporated in the glycoprotein's final oligosaccharide, is needed for target and recognition on lysosomal proteins such as NaGlu. The M6P on these oligosaccharides is specifically recognized by M6P receptors present on the cell surface. Thus, when M6P is omitted from the lysosomal enzymes such as NaGlu, the proteins are not recognized by the receptors and fail to be targeted to the lysosome. Thus, M6P is an effective epitope for the targeting of glycoproteins to the lysosomes.

Analysis of protein glycosylation is an important part of glycoprotein characterization. Oligosaccharides can be linked to a protein through a serine or a threonine as O-lined glycans or through an asparagine as N-linked glycans.

To analyze the structure of oligosaccharides, various chromatographic and spectroscopic techniques were performed. High-performance anion exchange chromatography with pulsed amperometric detection (HPAEC-PAD) was employed. Using this technique, oligosaccharides were quickly separated into general groups (i.e., neutral, singly charged, or multiply charged) and their structures were determined by comparison to pure standards.

All methods were based on protocols described by Hardy and Townsend (Hardy, M. R., and Townsend, R. R., “High-pH anion-exchange chromatography of glycoprotein-derived carbohydrates”, 1994, Methods Enzymol. 230: 208-225). Purified samples of transgenic avian derived rhNaGlu were dialyzed using a Tube-O-Dialyzer against nanopure water at 4° C. for about 24 hours to remove salts and other contaminants. Nanopure water was replaced four times during the entire dialysis period. After dialysis, each of the samples was divided into three aliquots. The aliquot intended for neutral and amino sugars analysis was hydrolyzed with 2 N trifluoroacetic acid (TFA) at 100° C. for 4 hours and the aliquot for mannose-6-phosphate analysis was hydrolyzed with 6.75 N TFA at 100° C. for 1.5 hours. The hydrolysates were then dried under N₂, re-dissolved with 50 μL H₂O, sonicated for 7 min in ice and transferred to an injection vial.

A mix of standards for neutral and amino sugars, and for mannose-6-phosphate with a known number of moles was hydrolyzed in the same manner and at the same time as the sample. Four different concentrations of the neutral and amino sugar standard mix and mannose-6-phosphate were prepared to establish a calibration equation. The number of moles of each sugar in the sample was quantified by linear interpolation from the calibration equation.

The oligossacharide profile and mannose-6-phosphate profile were analyzed separately by HPAEC-PAD. Instrument control and data acquisition were accomplished using Dionex chromeleon software. HPAEC-PAD analysis of hydrolyzed rhNaGlu detected M6P. The mean measured amount of M6P was 3.8 μg (CV 3.7%) per 210 μg of hydrolyzed protein. Converting to moles resulted in 13.4 nmol of M6P per 2.8 nmol of protein which was equivalent to a ratio of 3.2 moles of M6P per mole of protein.

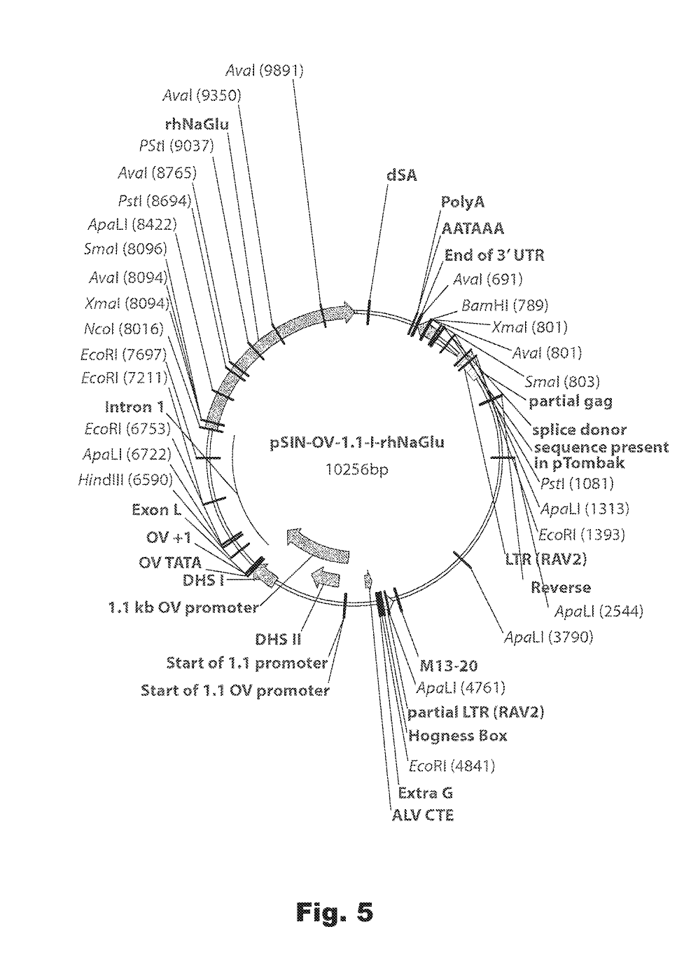

The oligosaccharide profile was also obtained for rhNaGlu (Gallus) using HPAEC-PAD (see FIG. 8). The profiles demonstrated good repeatability of the PNGase F reaction on the single sample. Peak clusters were observed in regions corresponding to neutral oligosaccharides (˜10 min to ˜20 min). A group of significantly smaller peaks eluting between ˜25 and ˜35 min were also observed, which were possibly attributed to singly charged species.

The monosaccharide composition analysis results obtained from samples of rhNaGlu produced from a transgenic avian (Gallus) are summarized in Table 3, which tabulates the average molar ratio of each monosaccharide analyzed for rhNaGlu.

TABLE 3
Monosaccharide Molar Ratios in rhNaGlu (Gallus)

| | |
|---|---|
| N-acetylgalactosamine (GalNAc) | 1.1* |
| N-acetylglucosamine (GlcNAc) | 35.6* |
| Galactose (Gal) | 4* |
| Mannose (Man) | 25.5* |
| Mannose-6-phosphate (M6P) | 3.2* |
| Fucose | Not detected |
| Glucose | Not detected |

*mole of monosaccharide per mole of protein

### Example 4

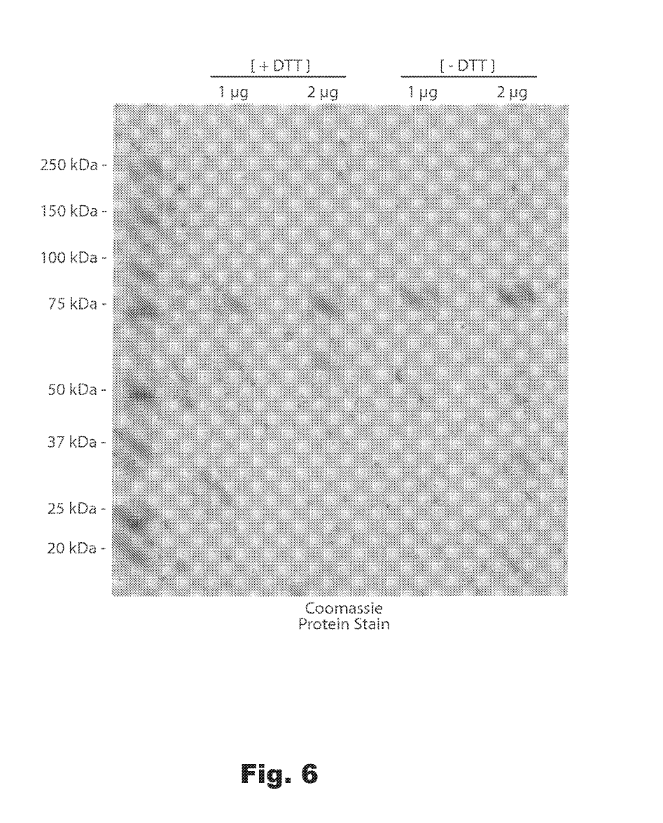

Wild-type human fibroblasts and mucopolysaccharidosis III B (NaGlu deficient) human fibroblasts were placed in a 24-well plate (2.5×10⁴ cells per well) and incubated for overnight at 37° C. in 5% CO₂. Conditioned media containing fibroblast basal medium and fibroblast growth kit having low serum were used. Various amounts of rhNaGlu (30, 10, 3.0, 1.0, 0.3 and 0 μg/mL) were co-incubated for 24 hours at 37° C. with 5% CO₂ to determine levels of cellular uptake by the human fibroblasts (see, FIG. 9). The wells were washed three times with PBS. 1004, lysis buffer was added per well and the plate was incubated for 10 min at 37° C. Cell lysate was transferred into 1.5 mL centrifuge tube. One cycle of freezing and thawing was performed. The cell lysate was centrifuged at 10,000 rpm for 10 min. 254, of supernatants were used for the assay. The assay time was 2 hours. The enzyme activity was measured using the methods known in the art and according to the methods described in Marsh et al., Clinical Genetics (1985) 27: 258-262, Chow et al., Carbohydrate Research (1981) 96:87-93; Weber et al., Protein Expression and Purification, (2001)21:251-259).

As shown in FIG. 9, negative control (i.e., MPS IIIB) did not exhibit any NaGlu activity while positive control (i.e., wild-type human fibroblast) showed NaGlu activity. MPS IIIB cells treated with 0.3 μg/mL of rhNaGlu exhibited approximately 50% of the normal activity level observed in wild-type fibroblast cells. MPS IIIB cells treated with 1 μg/mL of rhNaGlu demonstrated NaGlu activity that was approximately 4-fold higher than that observed in wild-type cells. Surprisingly, MPS IIIB cells treated with 30 μg/mL of rhNaGlu showed NaGlu activity that was at least 40-fold higher than that observed in wild-type cells. This result indicated that rhNaGlu produced from a transgenic avian (Gallus) was efficiently internalized into human fibroblasts at a high level.

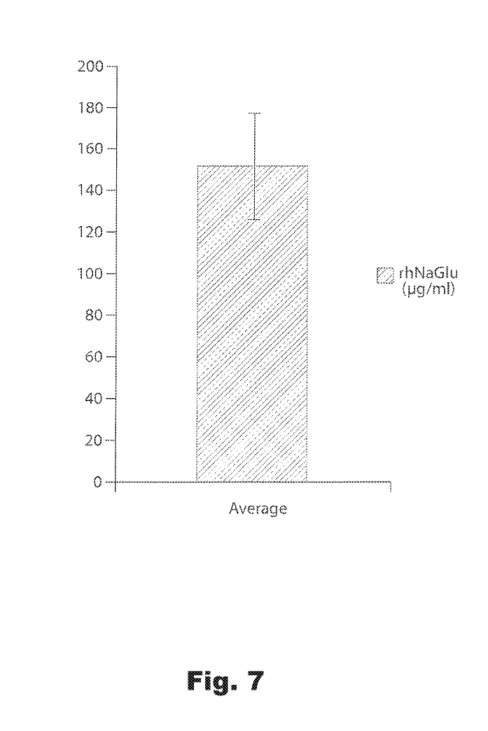

To determine whether internalization of rhNaGlu is via M6P receptor mediate endocytosis, M6P inhibition assays were performed. For the M6P inhibition assays, various concentrations of M6P were added to human MPS IIIB fibroblasts treated with 30 μg/mL of rhNaGlu and enzymatic activity was measured as described above. As shown in FIG. 10, human MPS IIIB fibroblasts did not exhibit any NaGlu activity as expected. MPSIII fibroblasts treated with 30 μg/mL of rhNaGlu with no M6P exhibited a high level of enzymatic activity, suggesting that the protein was efficiently internalized into the NaGlu deficient fibroblasts and retained activity. This enzymatic activity was inhibited by the presence of M6P monosaccharide in the medium at the concentration 0.03 mM and higher. The presence of 1mM of M6P monosaccharide in conditioned medium inhibited more than 90% of cellular uptake of the protein.

These results indicated that the rhNaGlu produced from a transgenic avian was efficiently internalized into the MPS IIIB fibroblasts via M6P receptor-mediated endocytosis and the rhNaGlu competed with M6P monosaccharides for the receptor recognition. The results were consistent with the glycan analysis that revealed the presence of the M6P structures on the rhNaGlu produced from the transgenic avian.

### Example 5

Two different rhNaGlu fusion constructs were designed to validate the feasibility of expressing rhNaGlu fusion proteins in the avian expression system.

In one construct, a nucleic acid sequence encoding 8 consecutive aspartic acid residues (DDDDDDDD) was fused to the nucleic sequence encoding NaGlu protein at the 5′ end of the full-length NaGlu cDNA sequence (SEQ ID NO:2) using conventional PCR and DNA recombinant technology. In another construct, a nucleic acid sequence encoding TfRL (i.e., THRPPMWSPVWP; SEQ ID NO:5) was fused to the nucleic sequence encoding NaGlu at the 3′ end of the full-length NaGlu cDNA sequence. The each construct was inserted into the pTT22 expression vector using EcoRI and HindIII restriction sites. The resulting vectors were each transfected into human embryonic kidney (HEK) 293 cells and stable clones expressing high levels of the fusion NaGlu proteins were obtained. An rhNaGlu protein fused to a stretch of 8 consecutive aspartic acid residues at N-terminus (AAA-NaGlu) and an rhNaGlu protein fused to transferrin receptor ligand (TfRL) at C-terminus (NaGlu-TfRL) were isolated from conditioned media.

The enzymatic activity of AAA-NaGlu and NaGlu-TfRL was measured using the methods known in the art (see, e.g., Marsh et al., Clinical Genetics (1985) 27:258-262; Chow et al., Carbohydrate Research, (1981) 96:87-93; Weber et al., Protein Expression and Purification (2001) 21:251-259; Neufeld et al., Protein Expression and Purification (2000) 19:202-211; and Weber et al., Human Molecular Genetics (1996) 5:771-777.

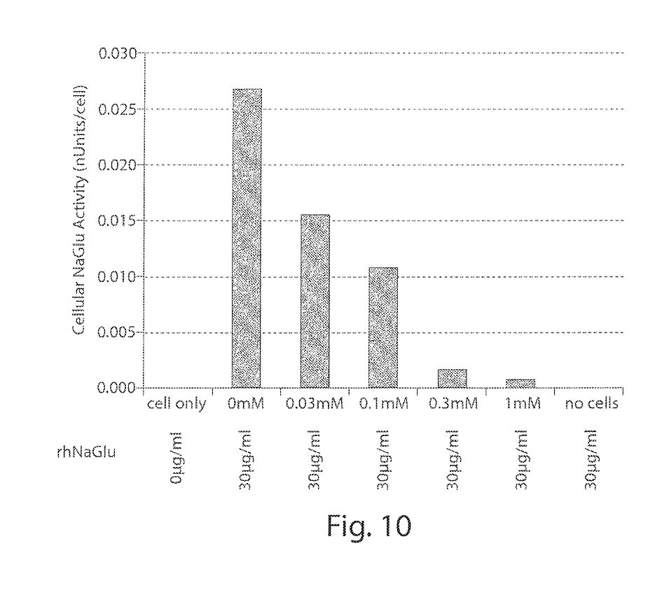

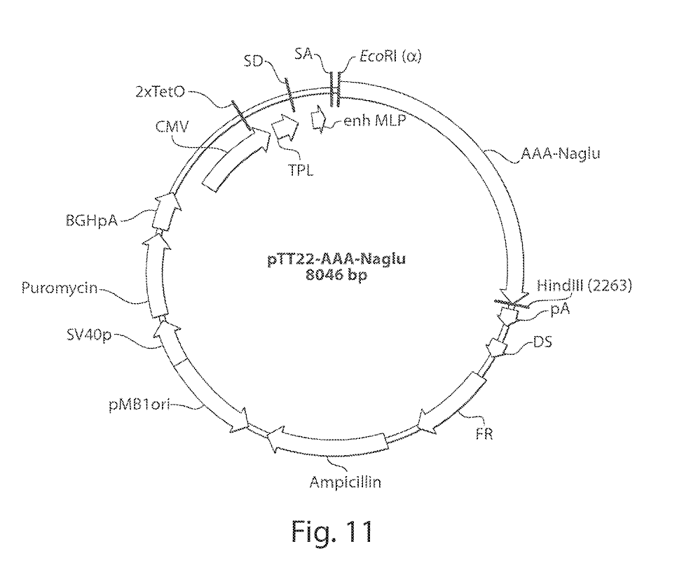

As shown in FIGS. 13 and 14, AAA-NaGlu and NaGlu-TfRL fusion proteins produced from HEK293 cells showed high levels of enzymatic activity. These results confirmed the possibility that these constructs can be used to produce NaGlu fusion proteins that have increased levels of phosphorylated mannose while retaining enzymatic activity from a transgenic avian expression system.

### Example 6

Cellular Uptake into Macrophages

Internalization of rhNaGlu produced from Gallus into human macrophage cells was also measured. NR8383 macrophage cells were incubated with 10μg/mL of rhNaGlu in F12 growth media for 0, 4, 8, 24, 32 and 48 hours at 37° C. with 5% CO₂. Samples were recovered and washed with PBS prior to lysis. 2.5×10⁵ cells were lysed in 1 mL of lysis buffer (10 mM of Na Phosphate pH6.0, 0.05% NP40), and lysates transferred into 1.5 mL centrifuge tubes and centrifuged at 10,000 rpm for 10 min. Protein concentration was determined by the Bradford assay and aliquots were frozen for NaGlu enzyme assays.

Enzyme activity was measured using standard methods. 25 mM of substrate (4-methylumbelliferyl 2-Acetamido-2-deoxy-a-D-glucopyranoside) was diluted to 2 mM in nanopure water to form a working substrate stock. Dilutions of samples were prepared in assay buffer (1% bovine serum albumin). 25 μL of 200 mM sodium acetate was distributed to wells of a multi-well plate. 254, of standard and 254, of samples were added to designated wells. 504, of the working substrate stock was added to each well and the plate was gently tapped to mix. The plate was sealed with adhesive film and incubated at 37° C. for 30 minutes. The reaction was then terminated by addition of 50 μL of stop solution (1M Glycine pH 12.5). The plate was placed on a microplate reader using a fluorescence bottom and the intensity was measured at an excitation 360nm and an emission 460 nm. The level of liberated 4-methylumbelliferone (4-MU) was measured by comparison with standards of 4-MU at 0.25 mM, 0.125 mM, 0.0625 mM, 0.0312 mM, 0.0156 mM, and 0.0078 mM.

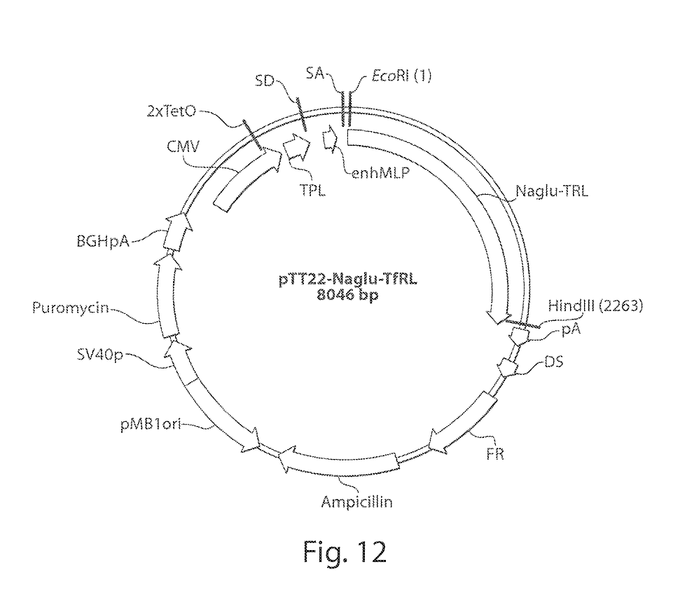

As depicted in FIG. 15, levels of the NaGlu activity in macrophages incubated with 10 μg/mL of rhNaGlu increased almost linearly over a 48 hour period: The rhNaGlu uptake by macrophages was rather slow, but steady throughout the entire time period measured. The relatively slow, extended uptake of NaGlu activity (as compared to other lysosomal enzymes containing M6P and/or mannose in their glycosylation structures) was unexpected and surprising. Equally surprising and unexpected was that a large amount of rhNaGlu proteins was taken up into the macrophages over the extended time period, resulting in intracellular enzymatic activity levels at least 10, 50, 100, 200, 300, 500, or even 1,000-fold higher than the basal levels observed in wild-type macrophages not exposed to rhNaGlu. The results demonstrate that rhNaGlu is extremely stable in extracellular as well as intracellular environments. Further, these results suggest that rhNaGlu may possess physicochemical characteristics that allow for longer serum half-life (e.g., longer circulation) and high serum concentrations in vivo, properties which are ideal for enhanced uptake into the central nervous system (CNS).

TABLE 4
Summary of NaGlu Characteristics

| | Avian (gallus) | Natural | |
|---|---|---|---|
| | produced | human | CHO produced |
| | rhNaGlu | NaGlu | human NaGlu |
| Apparent Molecular | ~85-~90 | ~86 | ~79-~89 |
| Mass (kDa) | | | |
| Enzymatic Activity | >1,000 | ~500 | ~1,057 |
| (nmol/min/mg) | | | |
| Mannose-6-phosphate | High | High | None or very |
| | | | Low |

### Example 7

Homozygous null mice were generated from breeding pairs of the strain B6.129S6-NaGlu^{tmlEfn}/J. Control wild-type mice were generated in the same manner. Genotyping was performed according to a standard PCR protocol. It is described in the art that at birth, homozygous naglu(^{-/-}) null mice are viable, normal in size, and do not display any gross physical or behavioral abnormalities, though they exhibited no NaGlu in all tissues (see, Li et al., (1999) Proc., Natl. Acad. Sci. USA 96:14505-14510). At one month of age, vacuolated macrophages are found in most tissues. Epithelial cells in kidney and neurons in some parts of the brain are also affected. The vacuolation becomes more prominent with age. At 4-5 months, the mice show abnormal behavior in an open field test. Older animals may have urinary retention and difficulty walking. Typical life span of the homozygous null naglu^{-/31} mice is 8-12 months (see, Li et al., (1999) Proc., Natl. Acad. Sci. USA 96:14505-14510). Intravenous (IV) administration

The intravenous administration of test article and vehicle by tail vein injection was accomplished as follows. Before injection vasodilation was achieved by gently warming the animal with an incandescent lamp or by soaking the tail in warm water, approximately 43° C. The animal was then placed in restraint device. The surface of the tail was disinfected with 70% isopropanol prior to injection. The lateral veins of the tail are located just under the skin and are identified in the distal part of the tail with the application of tension. A 27G needle, bevel up, was inserted into the vein for 3-4 mm. The test article or vehicle was then administered as a slow bolus injection over a period of ten seconds as evidenced by the observed clearing of the vein as the administered liquid momentarily occupies the vascular space. After removal of needle, gentle pressure was applied to the puncture site to provide hemostasis. The animal was monitored immediately following procedure to assure normal activity.

### Intrathecal (IT) Administration

The intrathecal administration of test article and vehicle by lumbar puncture injection was accomplished as follows. Before injection, animals were anesthetized using isoflurane that was maintained via nose cone throughout the procedure. The site of injection was prepared by shaving the fur, as necessary, prior to each injection. The animal was placed in a prone position on a platform, ensuring the hind limbs were straddling the platform forming a convex curve of the animals back. The surface of the back was swabbed with 70% isopropanol and allowed to dry prior to injection. Spinal column and hip bones were palpated to locate the L4-L5 or L5-L6 margin. A 30G needle, bevel facing cranially, was inserted into the intervertebral space. Placement was confirmed by the observation of a tail flick. The test article or vehicle was then administered as bolus injection. The animal was allowed to recover from anesthesia and monitored immediately following procedure to assure normal activity and use of limbs.

### Results

Twelve-week old naglu^{-/-} mice (B6.129S6-Naglu^{tmlEfn}/J) were administered rhNaGlu (Gallus) at dose levels of 6.75 or 27 mg/kg via tail vein injection (IV administration), once every other day, for a total of 5 doses at NaGlu concentrations of 1.125, or 4.5 mg/mL, respectively. Similarly, twelve-week old naglu^{-/-} mice were administered with rhNaGlu (Gallus) at a dose level of 0.31 mg/kg via lumbar puncture injection (IT administration), once every other day, for a total of 5 doses at NaGlu concentrations of 1.54 mg/mL. Vehicle (10 mM phosphate buffer, 150 mM NaCl and 0.02% Tween80, pH 5.5-5.8) were administered to naglu^{-/-} knock-out mice at the same dose concentration for 5 doses every other day. Untreated wild-type and naglu^{-/-} knock-out mice were also maintained for the duration of the study.

Animals were sacrifice at 4 hours after the fifth and final injection. All animals were necropsied and the liver, brain, spleen, heart, lung and kidneys were excised. Each organ was divided sagittally, providing samples for both frozen (−80° C.) and formalin-fixed storage.

Tissue samples were analyzed for: (1) heparan sulfate concentration using an analytical method based on SAX-HPLC analysis of heparan sulfate disaccharides; and (2) α-N-acetylglucosaminidase enzyme activity using a cell-based enzyme activity assay.

Histopathologic evaluation of brain, liver, kidney, spleen, heart and lung tissue was conducted using formalin-fixed tissue samples, embedded in paraffin, sectioned at 4μm, mounted on glass slides and stained with hematoxylin and eosin (H&E).

Following the repeated intravenous administration (5 doses over a 10 day period) of rhNaglu (Gallus) to naglu^{-/-} mice at dose levels of 6.25 and 27 mg/kg body weight, there was an apparent dose-dependent decrease in the concentration of Heparan Sulfate in the brain, liver and kidney of naglu^{-/-} mice (Table 5). The relative α-N-acetylglucosaminidase activity was increased in the brain and liver following intravenous administration (Table 6). These results were unexpected and surprising because the NaGlu enzymatic activities and resulting substrate clearance were observed in the brain of the treated naglu^{-/-} mice even with IV administration, suggesting that NaGlu (Gallus) administered intravenously was distributed to the brain of the treated naglu^{-/-} mice and effective to elicit efficacy even in the present of the blood brain barrier (BBB).

Following the intravenous administration (5 doses over a 10 day period) of rhNaGlu (Gallus) to naglu^{-/-} mice at a dose level of 0.31 mg/kg, there was a decrease in the concentration of Heparan Sulfate in the brain of naglu^{-/-} mice (Table 5), suggesting that rhNaGlu (Gallus) was properly targeted to the brain and effective in reducing the accumulated substrate in the brain of naglu^{-/-} mice.

TABLE 5
Tissue Substrate Level (rhNaGlu Gallus)

| | | | | | | | Heparan | | |
|---|---|---|---|---|---|---|---|---|---|
| | | | Age at | | | | Sulfate | | |
| | Animal | | sacrifice | | Dose | | ug/mg | | |
| Tissue | Number | Genotype | (wks) | Treatment | (mg/kg) | Route | tissue | mean | sd |
| KIDNEY | 253 | WT | 4 | na | — | — | 0.1 | | |
| | 155 | WT | 12 | na | — | — | 0.045 | 0.0725 | 0.038891 |
| | 178 | KO | 12 | na | — | — | 1.882 | | |
| | 242 | KO | 4 | na | — | — | 1.687 | | |
| | 145 | KO | 13 | na | — | — | 1.904 | | |
| | 474 | KO | 13 | vehicle | 0 | IV | 1.501 | | |
| | 479 | KO | 13 | vehicle | 0 | IV | 1.983 | | |
| | 484 | KO | 13 | vehicle | 0 | IV | 1.839 | 1.799333 | 0.175908 |
| | 487 | KO | 13 | rhNaGlu | 6.25 | IV | 0.928 | | |
| | 492 | KO | 13 | rhNaGlu | 6.25 | IV | 0.737 | 0.8325 | 0.135057 |
| | 481 | KO | 13 | rhNaGlu | 27 | IV | 0.591 | | |
| | 485 | KO | 13 | rhNaGlu | 27 | IV | 0.311 | | |
| | 490 | KO | 13 | rhNaGlu | 27 | IV | 0.585 | 0.495667 | 0.159954 |
| | 86 | KO | 15 | vehicle | 0 | IT | 2.105 | | |
| | 91 | KO | 14 | vehicle | 0 | IT | 1.704 | 1.9045 | 0.28355 |
| | 94 | KO | 14 | rhNaGlu | 0.31 | IT | 1.324 | | |
| | 101 | KO | 14 | rhNaGlu | 0.31 | IT | 2.233 | 1.7785 | 0.64276 |
| LIVER | 253 | WT | 4 | na | — | — | 0.045 | | |
| | 155 | WT | 12 | na | — | — | 0.092 | 0.0685 | 0.033234 |
| | 243 | WT | 4 | na | — | — | 0.045 | | |
| | 178 | KO | 12 | na | — | — | 1.85 | | |
| | 242 | KO | 4 | na | — | — | 2.263 | 2.0565 | 0.292035 |
| | 255 | KO | 4 | na | — | — | 1.85 | | |
| | 474 | KO | 13 | vehicle | 0 | IV | 1.822 | | |
| | 479 | KO | 13 | vehicle | 0 | IV | 1.981 | | |
| | 484 | KO | 13 | vehicle | 0 | IV | 2.004 | 1.961667 | 0.165779 |
| | 487 | KO | 13 | rhNaGlu | 6.25 | IV | 0.748 | | |
| | 492 | KO | 13 | rhNaGlu | 6.25 | IV | 0.444 | | |
| | 504 | KO | 13 | rhNaGlu | 6.25 | IV | 0.494 | 0.562 | 0.163009 |
| | 481 | KO | 13 | rhNaGlu | 27 | IV | 0.491 | | |
| | 485 | KO | 13 | rhNaGlu | 27 | IV | 0.172 | 0.3315 | 0.225567 |
| BRAIN | 253 | WT | 4 | na | — | — | 0.021 | | |
| | 155 | WT | 12 | na | — | — | 0.013 | | |
| | 243 | WT | 4 | na | — | — | 0.014308 | | |
| | 10 | WT | 36 | na | — | — | 0.012649 | 0.015239 | 0.003906 |
| | 239 | KO | 4 | na | — | — | 0.095 | | |
| | 178 | KO | 12 | na | — | — | 0.084 | | |
| | 242 | KO | 4 | na | — | — | 0.099 | | |
| | 255 | KO | 4 | na | — | — | 0.094538 | | |
| | 165 | KO | 24 | na | — | — | 0.084015 | | |
| | 474 | KO | 13 | vehicle | 0 | IV | 0.085447 | | |
| | 479 | KO | 13 | vehicle | 0 | IV | 0.072 | | |
| | 484 | KO | 13 | vehicle | 0 | IV | 0.073 | 0.085875 | 0.009972 |
| | 487 | KO | 13 | rhNaGlu | 6.25 | IV | 0.045 | | |
| | 492 | KO | 13 | rhNaGlu | 6.25 | IV | 0.044119 | | |
| | 504 | KO | 13 | rhNaGlu | 6.25 | IV | 0.044 | 0.044373 | 0.000546 |
| | 481 | KO | 13 | rhNaGlu | 27 | IV | 0.017796 | | |
| | 485 | KO | 13 | rhNaGlu | 27 | IV | 0.016668 | | |
| | 490 | KO | 13 | rhNaGlu | 27 | IV | 0.028 | 0.020821 | 0.006242 |
| | 86 | KO | 15 | vehicle | 0 | IT | 0.094521 | | |
| | 91 | KO | 14 | vehicle | 0 | IT | 0.072623 | 0.083572 | 0.015484 |
| | 94 | KO | 14 | rhNaGlu | 0.31 | IT | 0.038866 | | |
| | 101 | KO | 14 | rhNaGlu | 0.31 | IT | 0.028229 | 0.033548 | 0.007521 |

na: Not applicable (mice were untreated).

TABLE 6
Tissue enzymatic activity (rhNaGlu Gallus; U/ng protein)

| | | | | | | | Enzymatic |
|---|---|---|---|---|---|---|---|
| | | | Age at | | | | Activity |
| | Animal | | sacrifice | | Dose | | (U/ug |
| Tissue | Number | Genotype | (wks) | Treatment | (mg/kg) | Route | protein) |
| BRAIN | 253 | WT | 4 | na | — | — | 7.7 |
| | 178 | KO | 12 | na | — | — | 0 |
| | 474 | KO | 13 | vehicle | 0 | IV | 0 |
| | 479 | KO | 13 | vehicle | 0 | IV | 0 |
| | 484 | KO | 13 | vehicle | 0 | IV | 0.575 |
| | 487 | KO | 13 | rhNaGlu | 6.25 | IV | 10.58 |
| | 492 | KO | 13 | rhNaGlu | 6.25 | IV | 5.066666667 |
| | 504 | KO | 13 | rhNaGlu | 6.25 | IV | 4.033333333 |
| | 481 | KO | 13 | rhNaglu | 27 | IV | 87.91666667 |
| | 485 | KO | 13 | rhNaGlu | 27 | IV | 90.15 |
| | 490 | KO | 13 | rhNaGlu | 27 | IV | 17.35 |
| LIVER | 253 | WT | 4 | na | — | — | 36.69 |
| | 178 | KO | 12 | na | — | — | 0 |
| | 474 | KO | 13 | vehicle | 0 | IV | 0 |
| | 479 | KO | 13 | vehicle | 0 | IV | 0 |
| | 484 | KO | 13 | vehicle | 0 | IV | 0 |
| | 487 | KO | 13 | rhNaGlu | 6.25 | IV | 512.92 |
| | 492 | KO | 13 | rhNaGlu | 6.25 | IV | 378.805 |
| | 504 | KO | 13 | rhNaGlu | 6.25 | IV | 607.9225 |
| | 481 | KO | 13 | rhNaGlu | 27 | IV | 659.6825 |
| | 485 | KO | 13 | rhNaGlu | 27 | IV | 654.2475 |
| | 490 | KO | 13 | rhNaGlu | 27 | IV | 677.8725 |

na: not applicable (mice were untreated).

### Example 8

### CNS Penetration of rhNaGlu in Non-Human Primates

This Example describes an investigation of the CNS penetration of rhNaGlu in non-human primates.

rhNaGlu was administered to a normal M fascicularis by intrathecal injection at doses of 3 and 30 mg/dose on Days 1 and 8, followed by intravenous infusion at 3 or 30 mg/kg/dose on

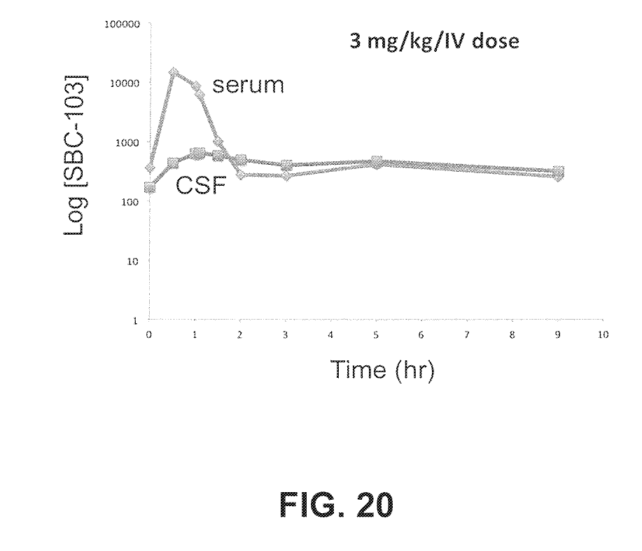

Days 15 and 22. This dose was well tolerated in the monkeys. Enzyme levels were measured over the following 9 hours. As shown in FIG. 20, an increase in rhNaGlu concentration was seen in the CSF on both Days 15 and 22, demonstrating CNS penetration of the IV-administered rhNaGlu.

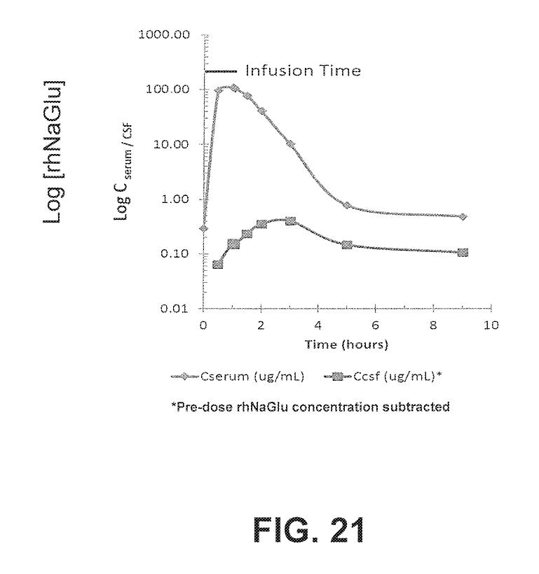

In additional experiments, a single lhour IV infusion was given at 30 mg/dose followed by measuring enzyme activity over the next 9 hours. 6 samples were tested at each time point: 3 animals×2 weekly doses. The concentration of rhNaGlu in the CSF following IV infusion shown in FIG. 21 demonstrates CNS penetration of the IV-administered rhNaGlu.

### Example 9

### Clinical Evaluation of rhNaGlu in Humans

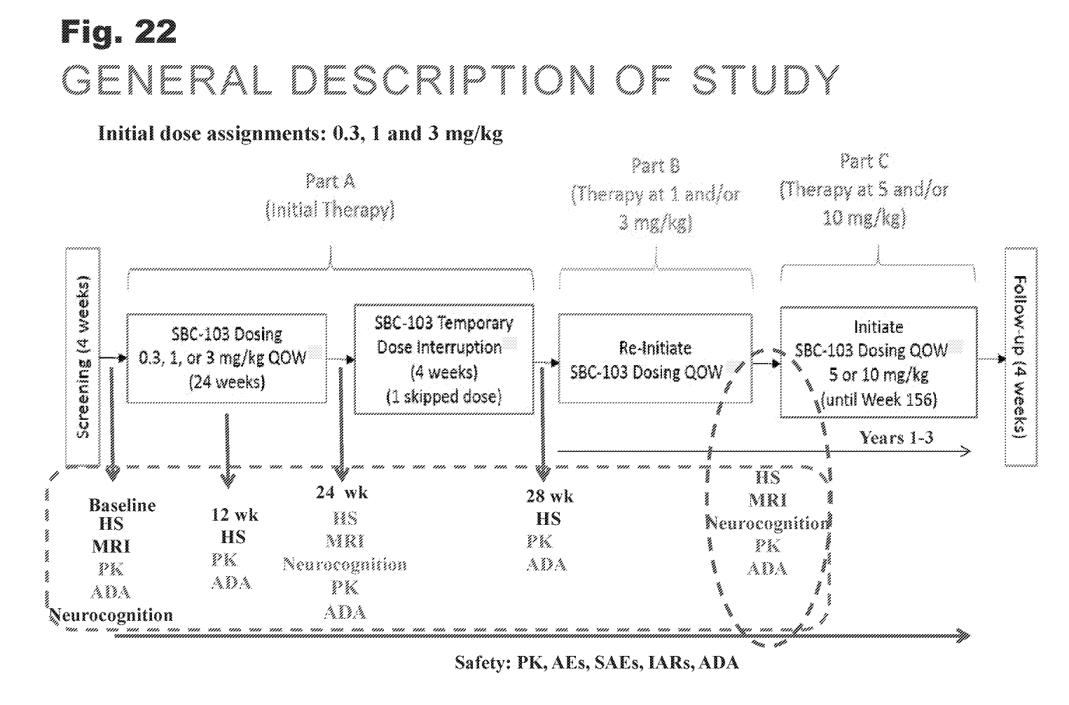

A clinical study, designated herein as NGLU-CL02, to evaluate the safety, pharmacokinetics, and pharmacodynamics/efficacy of intravenous (IV) administration of recombinant human NaGlu in subjects with mucopolysaccharidosis III, type B (MPS IIIB, Sanfilippo B) with evaluable signs or symptoms of developmental delay. The study design (comprising consecutive Parts A, B and C) is shown schematically in FIG. 22.

In Part A of the study, eligible patients received recombinant human NaGlu (referred to as SBC-103 in this study) at one of three different dosing cohorts: 0.3 mg/kg, 1 mg/kg, or 3 mg/kg every other week for 24 weeks. The study subjects (11 subjects) were 2-10 years of age and each had been diagnosed with MPS IIIM, with a mean age at diagnosis of 39 months (3 years) Six subjects were below 5 years of age; one was between 5 and 8, and four were older than 8. There were 4 female subjects and 7 male subjects. Ten of the eleven subjects were identified as rapid progressors (RP); one subject (008) was identified as a slow progressor (SP).

Prior to administering the first dose of recombinant human NaGlu, baseline levels of the following were obtained:

1) heparan sulfate levels in the cerebral spinal fluid (CSF) and serum;

2) brain morphomety including the cerebral cortical gray matter volume; and

3) neurocognitive evaluation.

Some baseline parameters were evaluated again at 12 weeks. At the end of the 24 week dosing period, baseline parameters were again evaluated. The week 26 dose was skipped, and at week 28, various baseline levels were assessed again, as shown in FIG. 22.

Part B of the study followed Part A and commenced at week 28, with therapy re-initiated at dosing levels of 1 mg/kg or 3 mg/kg SBC-103 QOW. Table 7 shows the length of participation in Part B and the transition to Part C. Subjects moved to Part C (a higher dose regimen, described below) after 16 to 36 weeks in Part B. All subjects received at least 11 QOW 3 mg/kg doses (range 11-25 doses) before transition to Part C. At the end of Part B, heparan sulfate levels, brain morphometry, neurocognition, pharmacokinetics (PK), and anti-drug antibody levels (ADA) were again assessed, as shown in FIG. 22.

TABLE 7
Time in Part B and Transition to Part C

| | | Week # in | |
|---|---|---|---|
| | Initial dose | Part B before | |
| | assignment | transition to | |
| Subject | (mg/kg) | Part C | Start of 3 mg/kg dose |
| 001 | 0.3 | Wk 64 | 16 Nov. 2015 (Wk 42) first dose 3 mg/kg, received ~11 doses |
| | | | before Part C |
| 002 | 0.3 | Wk 64 | 17 Nov. 2015 (Wk 42)first dose 3 mg/kg, received ~12 doses |
| | | | before Part C |
| 003 | 0.3 | Wk 64 | 11 Nov. 2015 (Wk 40) first dose 3 mg/kg, received ~12 doses |
| | | | before Part C |
| 004 | 1 | Wk 50 | 01 Oct. 2015 (Wk 28) first dose 3 mg/kg, received ~11 doses |
| | | | before Part C |
| 005 | 1 | Wk 50 | 28 Oct. 2015 (Wk 28) first dose 3 mg/kg, received ~12 doses |
| | | | before Part C |
| 006 | 1 | Wk 54 | 13 Oct. 2015 (Wk 28) first dose 3 mg/kg, received ~14 doses |
| | | | before Part C |
| 007 | 1 | Wk 54 | 05 Nov. 2015 (Wk 28) first dose 3 mg/kg, received ~14 doses |
| | | | before Part C |
| 008 | 3 | Wk 44 | 14 May 2015 (Wk 0) first dose 3 mg/kg, received ~22 doses |
| | | | before Part C |
| 009 | 3 | Wk 50 | 04 Jun. 2015 (Wk 0) first dose 3 mg/kg, received ~23 doses |
| | | | before Part C |
| 010 | 3 | Wk 50 | 02 Jun. 2015 (Wk 0) first dose 3 mg/kg, received ~16 doses |
| | | | before Part C |
| 011 | 3 | Wk 50 | 09 Jun. 2015 (Wk 0) first dose 3 mg/kg, received ~15 doses |
| | | | before Part C |

Part C of the study followed Part B, and commenced at different time points for different subjects as shown in Table 7. At the Part C baseline (PCBL), therapy was increased to 5 or 10 mg/kg QOW, with maintenance at that level expected until week 156 (up to 3 years).

During the clinical trial, the safety of the drug is monitored by assessing the occurrence of adverse events (AE), severe adverse events (SAE), and infusion-associated reactions (TAR). Anti-drug antibody (ADA) levels are monitored as part of the safety evaluation as well.

Pharmacokinetics (PK) were evaluated using an an enzymatic assay for the determination of SBC-103 that was fully validated in human serum at a detection limit=7 ng/mL.

Pharmacodynamics (PD) were evaluated by assessing heparan sulfate levels, cortical gray matter volume, and neurocognitive indicators over the course of SBC-103 administration. To determine heparan sulfate levels, samples (CSF or serum) were analyzed for heparan sulfate (HS) using liquid chromatograph-mass spectrometry (LC/MS/MS), with a detection limit of 5 ng/mL. Heparan sulfate digestion was carried out using heparinase I, II and III. Three major disaccharides were quantified: IV-A, IV-S and II-A, with abundances after hydrolysis of HS of about 83%, 10% and 2%, respectively. Total HS is determined by the addition of IV-A, II-A and IV-S disaccharides.

Brain morphometry studies involved structural magnetic resonance imaging (MRI) to obtain high resolution structural images of subject's brains. Structural MM assesses macroscopic effects of brain tissue degeneration such as tissue volume and atrophy measurements. Structural MRI parameters evaluated include cerebral cortical gray matter (GM) volume.

Neurocognitive evaluation made use of tests that measure cognitive ability, adaptive function, and other domains, that have an appropriate range of difficulty for MPS III, that are widely used with age-equivalent scores (AEq, or AE), and that are psychometrically sound. The Bayley Scales of Infant Development-Third Edition (BSID-III) and Kaufman Assessment Battery for Children-Second Edition (KABC-II) (ee, Delaney et al., JIMD Rep. 2014, 13:129-137; DOI 10.1007/8904_2013_269; Nov. 5, 2013) were selected because they provide the advantages of universality of use, availability of age-equivalent scores for severely impaired children, nonverbal content as on the cognitive scale on the BSID and the nonverbal scale of the KABC-II for higher functioning children, and availability of supplementary language and motor assessment (both domains on the and some language on the KABC-II). The Vineland Adaptive Behavior Scales—Second Edition (VABS-II), a parent-reported outcome, was selected as an adaptive measure for the same reasons.

The BSID-III/ KABC-II served to ascertain the cognitive age equivalency (AEq) in months using data obtained from the cognitive domain of the BSID-III and the mean AEq of the nonverbal domains of the KABC-II (Delaney et al., JIMD Rep. 2014, 13:129-137; DOI 10.1007/8904_2013_269; Nov. 5, 2013). The developmental quotient (DQ) was calculated by dividing the cognitive AEq by the calendar age in months, multiplied by 100 (Delaney et al., JIMD Rep. 2014, 13:129-137; DOI 10.1007/8904_2013_269; Nov. 5, 2013).

Comparisons were made to the clinical course of mucopolysaccharidosis type IIIA (MPS IIIA) as reported in Shapiro et al., “A Prospective Natural History Study of Mucopolysaccharidosis Type IIIA”, J. Ped. 2016;170:278-87. That study followed 24 children for at least 12 months. The children ranged in age from 1.1 to 18.4 years old, 19 of whom were rapid progressing (RP) and 5 of whom were slow progressing (SP). The patients had confirmed diagnosis of MPS IIIA and calendar age as well as Vineland age equivalence of >1 year. In that Natural History (NH) study, it was found that rapid progressing (RP) children lost −9.8 DQ points/year (<6 years decline -14.6 points/year). Development plateaued by 30 months, followed by rapid regression after 40-50 months. In RP patients, cognitive development quotients (DQ) showed consistent steep declines associated with progressive cortical gray matter atrophy, and along with gray matter volume, were found to be sensitive markers of disease progression in MPS IIIA. These markers may have utility as clinical endpoints in treatment trial. It was concluded that for optimal outcomes, treatment may need to be instituted in children before the onset of steep cognitive decline and brain atrophy. In addition to being helpful for understanding the natural history and rate of disease progression in MPS IIIA patients, this data can also be used to support the estimation of likely rate of disease progression in a MPSIIIB patient population.

Comparisons were also made to the clinical course of mucopolysaccharidosis type TIM (MPS IBB) in a prospective natural history (NH) study (https://clinicaltrials.gov/ct2/show/study/NCT0150976).

### Results

Eleven patients were enrolled (median age 4 years; range 2-10 years) and received IV SBC-103. All 11 patients continue on extended therapy. During the initial 24 weeks, there were 3 treatment-emergent serious adverse events (SAEs) in a single patient and 6 infusion-associated reactions in 3 patients. No SAEs were considered related to SBC-103.

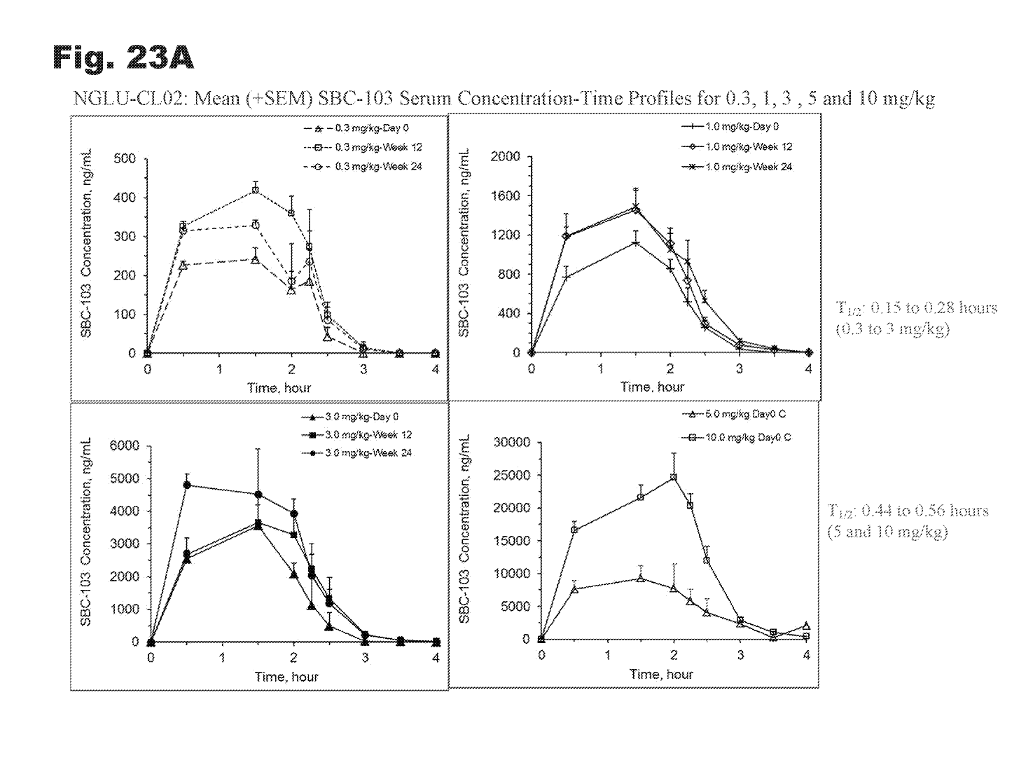

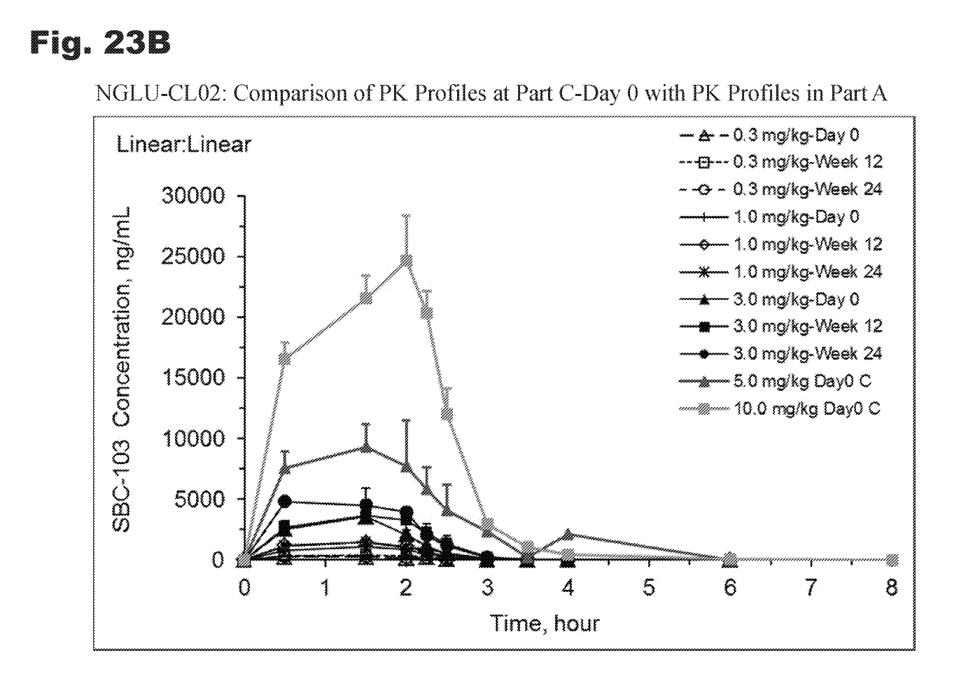

Pharmacokinetic (PK) data for dosing levels of 0.3 mg/kg, 1 mg/kg, 3.0 mg/kg (baseline, week 12, and week 24) as well as for dosing levels of 5.0 mg/kg and 10.0 mg/kg at Part C baseline, are shown in FIG. 23A. PK exposures increased as the dose was increased. SBC-103 PK exposure appears to be time dependent, and higher after multiple doses than after first dose. Superimposing the data (FIG. 23B) shows that PK exposure increased significantly at 5 and 10 mg/kg dose in Part C-Day 1, compared with PK exposures at lower doses from 0.3 to 3 mg/kg in Part A of the study.

Table 8 shows a summary of PK parameters.

TABLE 8
Summary of Selected Pharmacokinetic Parameters for SBC-103

| | Dose | | AUCₐₗₗ, | | | | | | | |
|---|---|---|---|---|---|---|---|---|---|---|
| VISIT | mg/kg | N | (ng · h/mL) | | Cₘₐₓ, (ng/mL) | | T_{1/2} (h) | | Tₘₐₓ (h) | |
| Day 0 | 0.3 | 3 | 446.14 ± 105 | (23.6%) | 250 ± 39 | (16%) | 0.28 ± 0.01 | (2.57%) (2) | 1.09 | (0.5-2.25) |
| BL | 1 | 4 | 1883 ± 198 | (10.5%) | 1072 ± 199 | (19%) | 0.22 ± 0.04 | (18%) (3) | 1.63 | (1.5-2.0) |
| | 3 | 4 | 5759 ± 1893 | (33%) | 3616 ± 1960 | (54%) | 0.15 ± 0.03 | (20%) (3) | 1.26 | (0.5-1.53) |
| Week | 0.3 | 3 | 737 ± 14 | (1.9%) | 433 ± 29 | (6.6%) | 0.22 ± 0.03 | (16%) (2) | 1.43 | (1.0-1.75) |
| 12 | 1 | 5 | 2609 ± 453 | (17%) | 1493 ± 461 | (31%) | 0.284 ± 0.10 | (34%) | 1.41 | (0.5-2.0) |
| | 3 | 3 | 6874 ± 1657 | (24.1%) | 3835 ± 854 | (22.3%) | 0.27 ± 0.12 | (46%) | 1.65 | (1.50-1.92) |
| Week | 0.3 | 3 | 608 ± 113 | (18.6%) | 333 ± 30 | (9.1%) | 0.18 | (1) | 1.65 | (1.5-1.92) |
| 24 | 1 | 4 | 2557 ± 790 | (31%) | 1372 ± 419 | (31%) | 0.30 ± 0.05 | (17.6%) | 1.81 | (1.50-2.50) |
| | 3 | 4 | 9265 ± 1807 | (19.5%) | 5298 ± 788 | (15%) | 0.25 ± 0.08 | (34%) | 1.38 | (0.5-−2.02) |
| Week | 3 | 5 | 10247 ± 2071 | (20%) | 5053 ± 1280 | (25%) | 0.33 | (1) | 1.33 | (0.58-1.55) |
| 52 | | | | | | | | | | |
| Day 0 | 5 | 5 | 20683 ± 6542 | (32%) | 9462 ± 2388 | (25%) | 0.44 ± 0.33 | (75%) (4) | 1.86 | (0.5-3.7) |
| Part C | 10 | 6 | 49289 ± 10785 | (22%) | 24730 ± 6544 | (26%) | 0.55 ± 0.22 | (40%) | 1.85 | (1.5-2.5) |

Mean ± SD (CV %) reported except for Tₘₐₓ (range);
n in the bracket indicates the sample size for T_{1/2}
Subject 10 received 1 mg/kg dose instead of 3 mg/kg at Week 12. This subject was grouped in 1 mg/kg

In the Part A, dose from 0.3 to 3 mg/kg, the mean T₁₁₂ for SBC-103 ranged from 0.15 to 0.28 hours; the mean T₁₁₂ appears independent of dose and duration of treatment. In Part B-Week 52, number of subjects and number of available PK time points were limited. In Part C-Day 1 at 5 and 10 mg/kg dose, mean T₁₁₂ ranged from 0.44 to 0.56 hours, and PK exposures increased more than dose increased from 5 to 10 mg/kg dose.

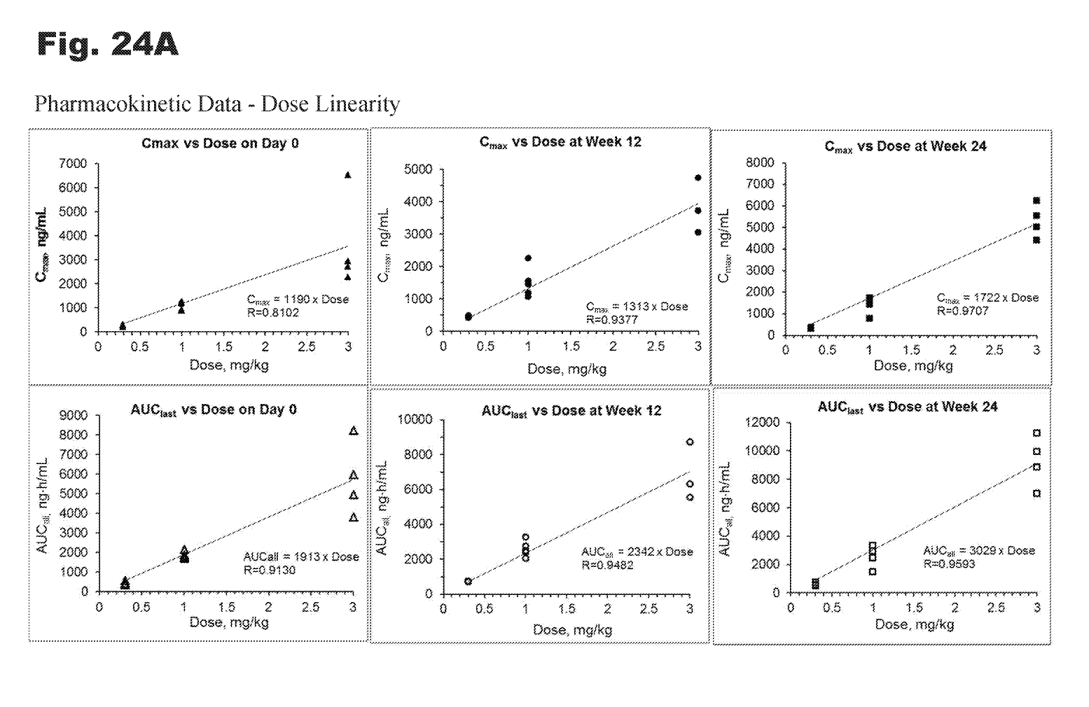

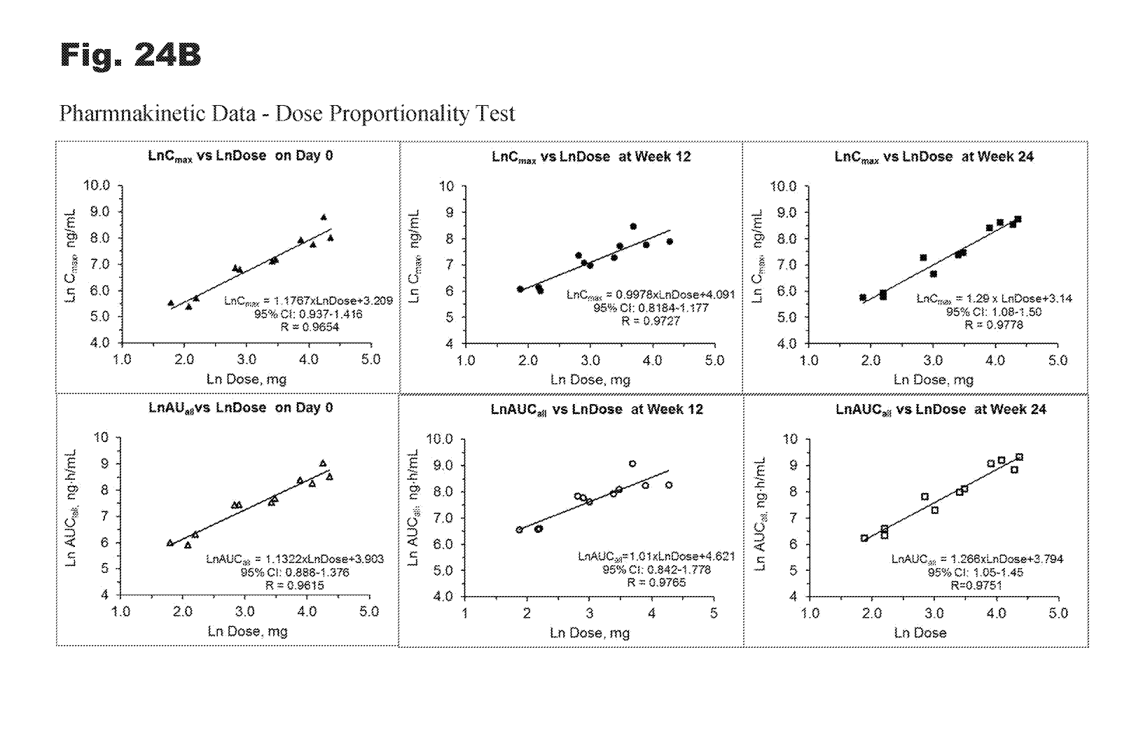

The PK analysis showed that SBC-103 had a serum half-life from 9 to 18 minutes for 0.3 to 3 mg/kg dosing levels, and 26 to 33 minutes for Part C baseline doses of 5 mg/kg and 10 mg.kg. PK exposure increased linearly with increase of dose (FIG. 24A), with Cₘₐₓ and AUCₐₗₗ linearly correlated with dose levels from 0.3 to 3 mg/kg on day 0, week 12 and week 24. PK exposure showed accumulation after multiple dose administration. At week 24 of the study, PK accumulation ratio ranged from 1.27 to 1.97-fold. PK exposures increased more than dose proportional after multiple dose administration at week 24 (FIG. 23A, FIG. 24B). On day 0 and week 12, PK exposures increased in a dose proportional manner from 0.3 to 3 mg/kg as shown by the 95% CI of the slope which included 1; at week 24, PK exposures increased in a more than dose proportional manner as shown by the slope of 1.3 and its 95% CI excluded 1.

To summarize the PK data, the mean T_{1/2} ranged from 0.15 to 0.28 hours and independent of dose levels at doses from 0.3 to 3 mg/kg; the mean T_{1/2} ranged from 0.44 to 0.56 hours at 5 and 10 mg/kg; PK exposure increased linearly with increase of doses from 0.3 to 3 mg/kg on day 0 and week 12; and PK exposure increased more than dose proportional at week 24 from 0.3 to 3 mg/kg. With respect to the 5 and 10 mg/kg PK on Day 1-Part C, PK exposures increased more than dose increase from 5 to 10 mg/kg.

Pharmacodynamic data, including total heparan sulfate in CSF and serum, were obtained through at least week 52.

A summary of pharmacodynamic results obtained through week 28 for CSF and serum heparan sulfate (HS) levels is shown in Table 9.

TABLE 9

| | Decrease | | | |
|---|---|---|---|---|
| | in CSF | | | |
| | Total HS | Decrease in Serum | No Change or | No Change or |
| Dose | Level from | Total HS | Increase in Cortical | Increase in BSID |
| Level | Baseline | Level from | GM Volume from | III/KABC-II DQ from |
| through | to 6 Months | Baseline to week 28 | BL to 6 Months | BL to 6 Months |
| week 28 | n/N (%) | n/N (%) | n/N (%) | n/N (%) |
| 0.3 mg/kg | 1/3 (33%) | 3/3 (100%) | 0/3 (0%) | 1/3 (33%) |
| 1 | 2/4 (50%) | 4/4 (100%) | 1/2 (50%)* | 2/4 (50%) |
| mg/kg** | | | | |
| 3 mg/kg | 4/4 (100%) | 4/4 (100%) | 2/4 (50%) | 3/4 (75%) |

*week 24 MRI data were not available for 2 subjects
**one subject moved to 3.0 mg/kg at week 24

After 24 weeks of treatment, total heparan sulfate percent change from baseline, reported asmean (SD), in the CSF was 10.9 (14.)], −0.43 (11.9) and −26.2 (20.9) for 0.3 mg/kg, 1 mg/kg, and 3 mg/kg groups, respectively. Total HS percent change from baseline, reported as mean (SD), in the serum was −39.6 (15.4), −53.9 (19.6) and −40.5 (23.9) for 0.3 mg/kg, 1 mg/kg, and 3 mg/kg groups, respectively. HS reduction in CSF was linearly correlated with SBC-103 serum PK exposures. An interim 12-week analysis from the Phase 1/2 study reported in December 2015 showed a dose-dependent reduction of HS levels in CSF in the three dosing cohorts (mean reductions of 3 percent, 6 percent and 11 percent at 0.3 mg/kg [N=3], 1.0 mg/kg [N=4], and 3.0 mg/kg [N=3], respectively). One patient who received only one 3 mg/kg and one 1 mg/kg dose (instead of 6 doses at 3 mg/kg) due to an unrelated SAE was excluded from this analysis, and restarted treatment at week 8.

Subjects initially receiving 0.3 mg/kg were escalated to 1 mg/kg at week 28, and further escalated to 3 mg/kg at week 40-42. Total HS percentage change in CSF from baseline to Part C baseline, reported as mean (SD), was −2.10 (12.59) for initial dose assignment of 0.3 mg/kg (range: −10.9 to +12.3). Total HS percentage change in serum from baseline to Part C baseline, reported as mean (SD) was −59.32 (8.56) for initial dose assignment of 0.3 mg/kg, (range: −49.4 to −64.6).

Subjects initially receiving 1 mg/kg were escalated to 3 mg/kg at week 28. Total HS percentage change in CSF from baseline to Part C baseline, reported as mean (SD), was −4.15 (4.8) for initial dose assignment of 1 mg/kg (range: -8.6 to +0.26). Total HS percentage change in serum from baseline to Part C baseline, reported as mean (SD) was −56.67 (12.6) for initial dose assignment of 1 mg/kg, (range: −38.9 to −68.3).

Subjects initially receiving 3.0 mg/kg were not escalated through at least week 52. Total HS percentage change in CSF from baseline to Part C baseline, reported as mean (SD), in the CSF was −15.58 (4.84) for initial dose assignment of 3 mg/kg (range: −10.8 to −21.7). Total HS percentage change in serum from baseline to Part C baseline, reported as mean (SD), in the serum was −53.28 (7) for initial dose assignment of 3 mg/kg, (range: −42.9 to −57.4).

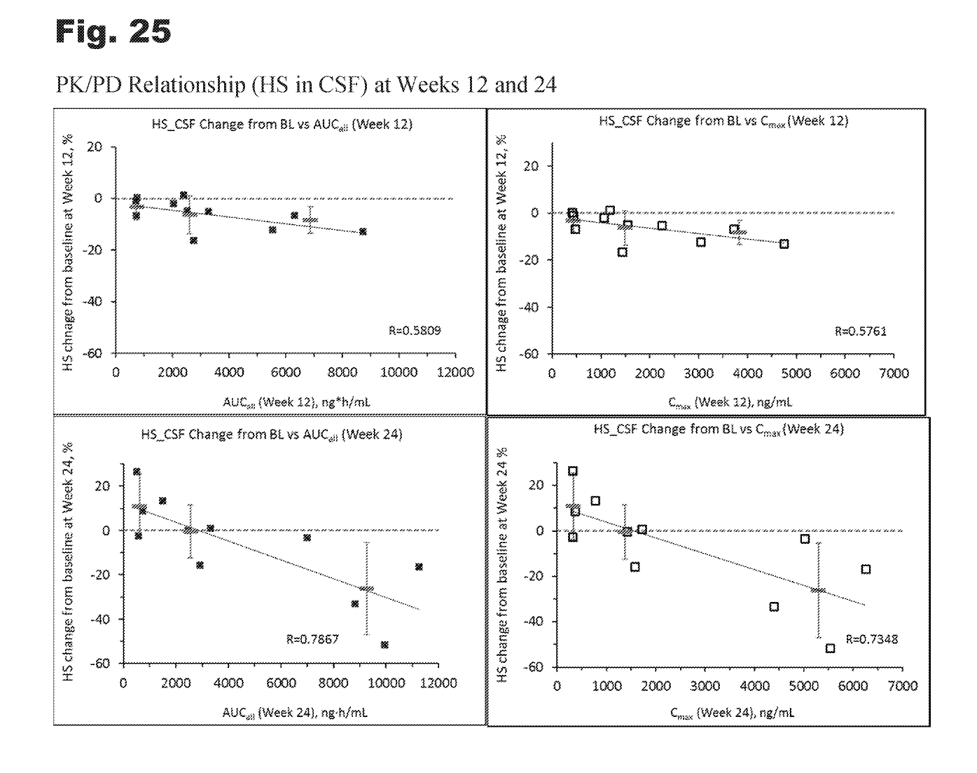

The data show that reductions in CSF heparan sulfate levels were observed in subjects from each dosing cohort, and that reduction was observed in all subjects in the 3 mg/kg cohort. At weeks 12 and 24, individual HS-CSF reduction data were linearly correlated with increase in serum PK exposures (Cₘₐₓ and AUCₐₗₗ) (FIG. 25) Correlation coefficient is moderate (R=0.58 at Week 12 and 0.73-0.79 at Week 24). HS-CSF reduction was variable at lower PK exposures (i.e., 0.3 mg/kg).

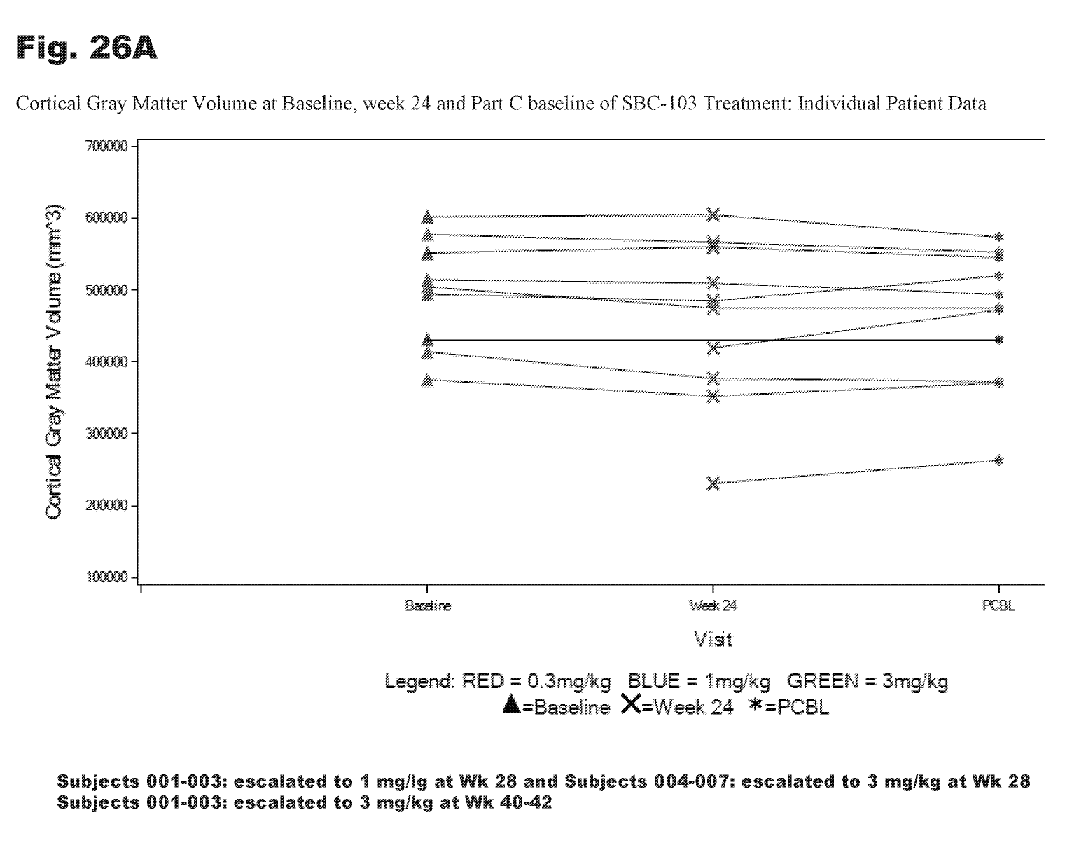

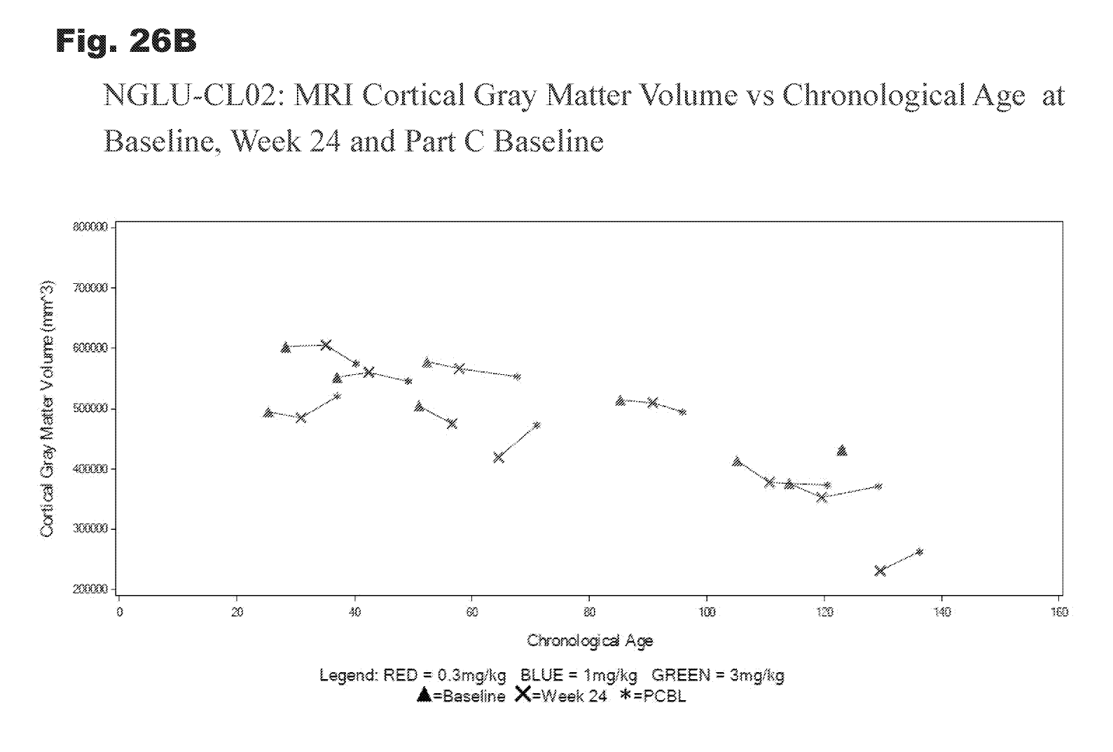

The reductions in CSF heparan sulfate levels were surprisingly accompanied by stabilization or improvement (i.e., no change, or an increase) in cortical gray matter volume (abbreviated as either cortical GMV or CGM volume) (Table 9; FIGS. 26A and 26B). Table 10 shows data for individual subjects.

TABLE 10
Cortical Gray Matter Volume at Baseline, week 24 and Part C baseline of SBC-103
Treatment: Individual Patient Data

| | Age at | Initial | | Change in | Change in |
|---|---|---|---|---|---|
| | baseline, | dose | CGM at | CGM at Part C | CGM at Part |
| Sub # | mo | (mg/kg) | baseline/wk24/Part C BL | BL (mm3) | C BL (%) |
| 004 | 36.5 | 1 | 551589.5/559943/545299.9 | −6289.6 | −1.14 |
| 007 | 27.6 | 1 | 602247.1/604795.7/574179.2 | −28067.9 | −4.66 |
| 009 | 24.7 | 3 | 494596.2/484851.5/520309.5 | +25713.3 | +5.20 |
| 010 | 51 | 3 | 504528.3/475242.5/NA | −29285.8* | −5.80* |
| 011 | 59 | 3 | NA/419426.3/472184.7 | +52758.4* | +12.58* |
| 001 | 51.9 | 0.3 | 577481/566254/552958.6 | −24522.4 | −4.25 |
| 002 | 114 | 0.3 | 375555.6/352898.7/370865.6 | −4690.0 | −1.25 |
| 003 | 105.1 | 0.3 | 413593.3/377192/373104 | −40489.3 | −9.79 |
| 005 | 123.4 | 1 | NA/230899.8/262868.9 | +31969.1* | +12.16* |
| 006 | 123 | 1 | 431193.8/NA/411722.2 | −19471.6 | −4.52 |
| 008 | 85 | 3 | 514020/509664/494018 | −20002.0 | −3.89 |

*Based on limited data due to uninterpretable/low quality score scans; changes calculated based on available time points

For comparison, the mean % decline in cortical gray matter (CGM) volume for Rapid Progressors (RP) in the MSPIIIA Natural History study after 1 year was 7.5%; mean % decline in CGM volume for patients in the MPS IIIB natural history study (<12 years) after 1 year was 7.8% (Shapiro et al., J. Ped. 2016; 170:278-87; Nestrasil et al., J Ped., 2016, 170:278-287.e4; Nestrasil et al., Eur. J. Ped. Neurolog. 19:S15-S16; Yund et al., 2015 February; 114(2):170-7). Compared with age-matched subjects in MPSIIIA database, GMV for most subjects in the clinical study are stable or slightly increase over 1 year; the mean (SD) % decline in CGM volume at Part C baseline for the MPSIIIB subjects in the clinical study was −3.2% (4.35). No subjects had GMV worse than MPSIIIA Natural History (NH) data (data not shown)

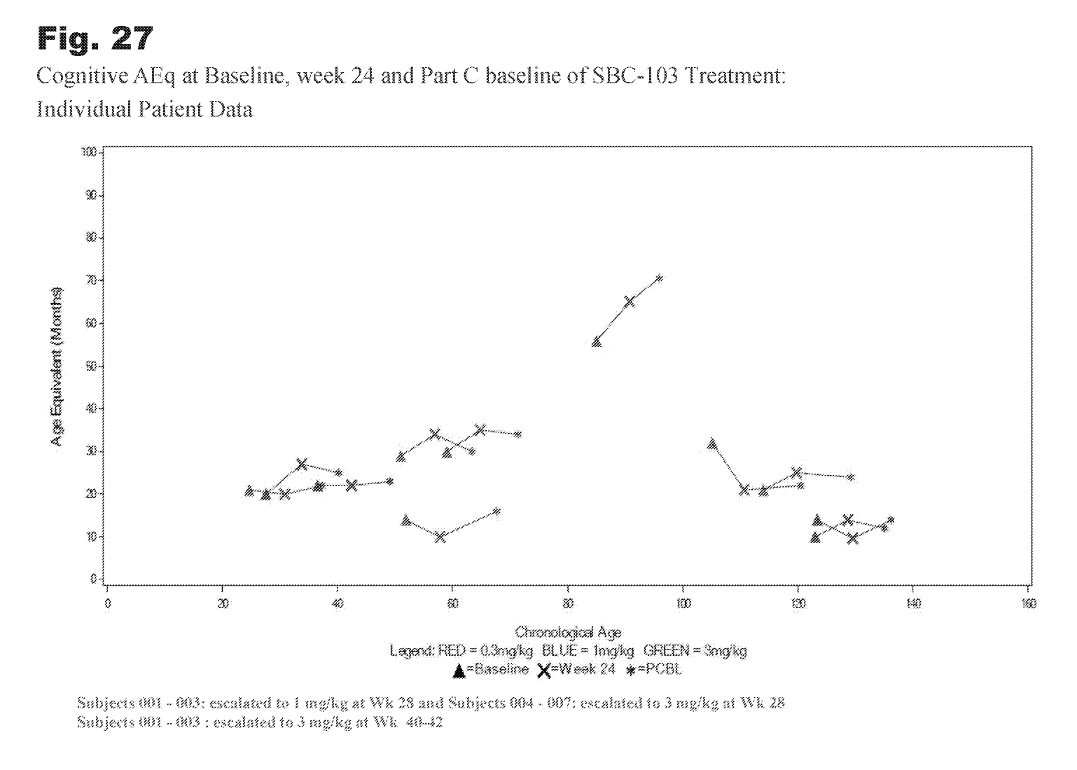

The reductions in CSF heparan sulfate levels were also surprisingly accompanied by improved neurocognitive performance as assessed using BSIDIII/KABC-II (Table 9). Cognitive Age Equivalence (AEq) plotted against chronological age is shown in FIG. 27 ; Table 11 shows data for individual subjects.

TABLE 11
Cognitive Age Equivalent at Baseline, week 24 and Part C
baseline of SBC-103 Treatment: Individual Patient Data

| | Age at | Initial | Cognitive AE, | Change in |
|---|---|---|---|---|
| | baseline, | dose | baseline/wk24/ | AE at Part |
| Sub # | mo | (mg/kg) | Part C BL | C BL |
| 004 | 36.5 | 1 | 22/22/23 | +1 |
| 007 | 27.6 | 1 | 20/27/25 | +5 |
| 009 | 24.7 | 3 | 21/20/22 | +1 |
| 010 | 51 | 3 | 29/34/30 | +1 |
| 011 | 59 | 3 | 30/35/34 | +4 |
| 001 | 51.9 | 0.3 | 14/10/16 | +2 |
| 002 | 114 | 0.3 | 21/25/24 | +3 |
| 003 | 105.1 | 0.3 | 32/21/22 | −10 |
| 005 | 123.4 | 1 | 14/9.7/14 | 0 |
| 006 | 123 | 1 | 10/14/12 | +2 |
| 008 | 85 | 3 | 56/65.2/70.6 | +14.6 |

Nine out of eleven (9/11) subjects showed increase in AEq over baseline, one subject showed no change and one subject declined. For comparison, the mean decline in AEq in Rapid Progressors (RP)<6 yrs in the MSPIIIA Natural History study after 1 year was 1.9 mo.; mean decline in AEq in all RP in the MPSIIIA Natural History study after 1 year was 1.6 mos. (Shapiro et al., J. Ped. 2016; 170:278-87). Compared with age-matched subjects in MPSIIIA database, there appears to be some treatment effect over 1 year (data not shown).

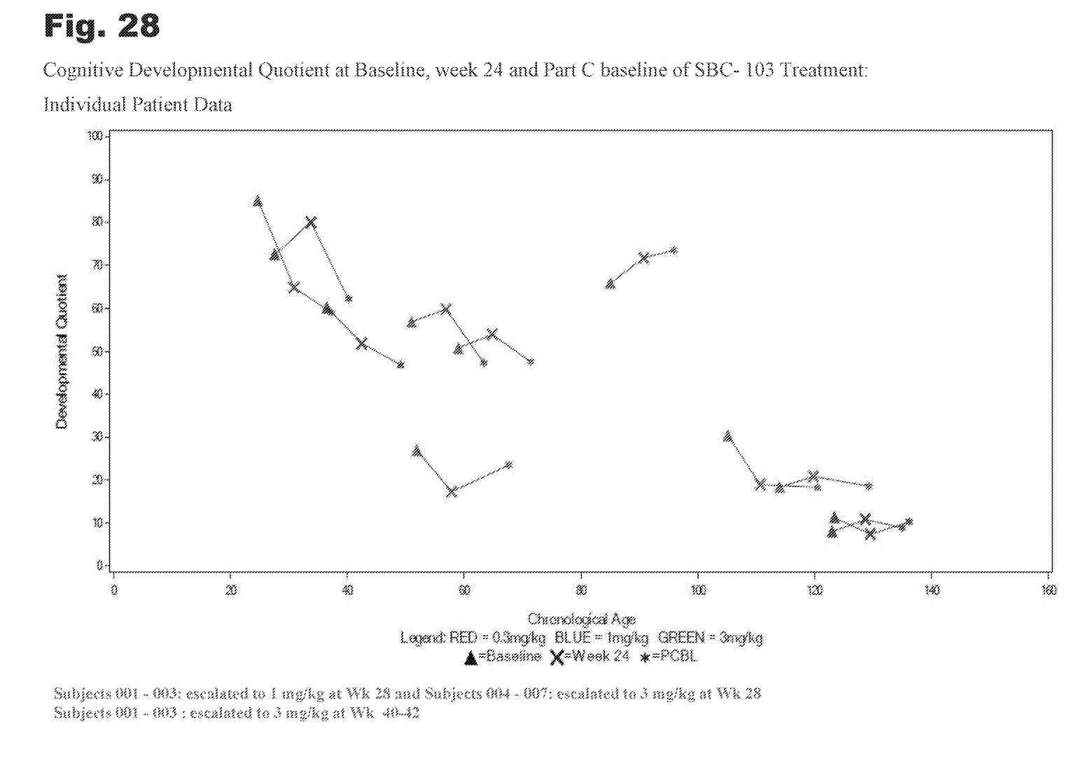

Developmental Quotient (DQ) plotted against chronological age is shown in FIG. 28 . Table 12 shows data for individual subjects.

TABLE 12
Cognitive DQ at Baseline, week 24 and Part C baseline
of SBC-103 Treatment: Individual Patient Data

| | Age at | Initial | Cognitive DQ, | Change in |
|---|---|---|---|---|
| | baseline, | dose | baseline/wk24/ | DQ at Part |
| Sub # | mo | (mg/kg) | Part C BL | C BL |
| 004 | 36.5 | 1 | 60.2/51.8/46.8 | −13.4 |
| 007 | 27.6 | 1 | 72.6/80/62.2 | −10.4 |
| 009 | 24.7 | 3 | 85.2/64.9/59 | −26.2 |
| 010 | 51 | 3 | 56.9/59.8/47.3 | −9.6 |
| 011 | 59 | 3 | 50.8/54/47.6 | −3.2 |
| 001 | 51.9 | 0.3 | 27/17.3/23.6 | −3.4 |
| 002 | 114 | 0.3 | 18.4/20.9/18.6 | +0.2 |
| 003 | 105.1 | 0.3 | 30.4/19/18.3 | −12.1 |
| 005 | 123.4 | 1 | 11.3/7.5/10.3 | −1 |
| 006 | 123 | 1 | 8.1/10.9/8.9 | +0.8 |
| 008 | 85 | 3 | 65.9/71.8/73.6 | +7.7 |

Ten out of eleven (10/11) subjects showed a decline in DQ of less than 15 and 20 points at Part C baseline. Subject 009 (24.7mo), however, nonetheless showed a 1 month AEq increase over baseline at Part C, despite the larger drop in DQ. For comparison, reference can be made to MPS IIIA and MPS IIIB Natural History studies. In 8 MPS IIIA RP subjects over 2 years of age and DQ more than 50 at baseline, 4/8 subjects had a decline in Age Equivalence (AEq) of 4-16 months (4, 7, 15 and 16), 2/8 subjects had no change in AEq (0 mo) and 2/8 subjects had increase in AEq (2 and 5 mo) after 2 years; 7/8 subjects had DQ decline of more than 30 DQ points (34, 34, 36, 36, 39, 45, 45) with 1/8 subject showing DQ decline of 28 points after 2 years. In 9 MPS IIIA RP subjects over 2 years of age and DQ over 50 at baseline, 4/9 subjects had AEq decline (5,7,8, 10 mo), 2/9 had no change in AEq (0 mo) and 3 had increase in AEq (1, 2 and 3 mo) after 1 year; 7/9 subjects had DQ decline of more than 20 DQ points (23, 23, 24, 26, 26, 28, 31) with 2/9 subject showing DQ decline of less than 20 points (11 and 16) after 1 year.

The mean decline in DQ in Rapid Progressors (RP)<6 yr in the MPSIIIA Natural History study in the first year was about 20 points, and the mean decline all RP in the MPS IIIA Natural History study in the first year was about 15 points (Shapiro et al., J. Ped. 2016; 170:278-87). Compared with age-matched subjects in MPSIIIA database, all subjects had decline of DQ over 1 year; however, the DQ decline did not seem to be worse than NH data (data not shown). As the clinical study continues, a positive treatment outcome may include DQ stabilization, which can be considered as no more than 15 point decline in DQ compared to baseline.

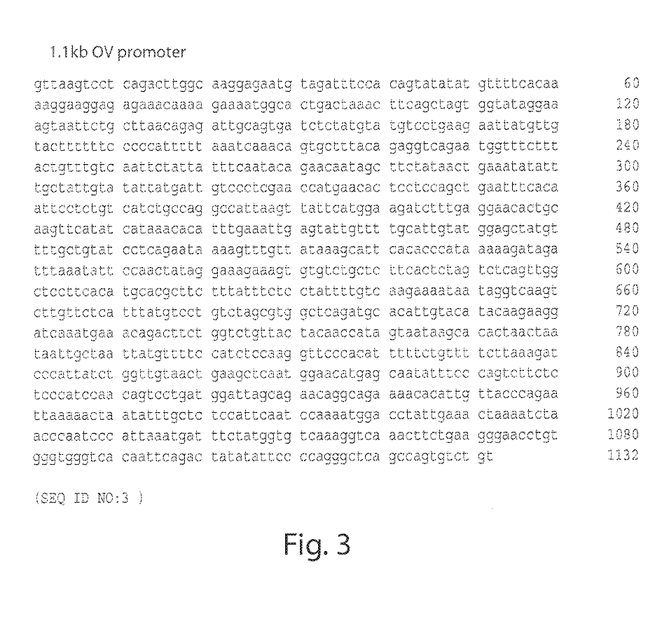

Notably, the decline of DQ within individuals correlates with the loss of grey matter volume, suggesting that grey matter volume change may be able to serve as an objective surrogate of clinical disease progression (see Shapiro et al., J. Ped. 2016; 170:278-87, FIG. 3B).

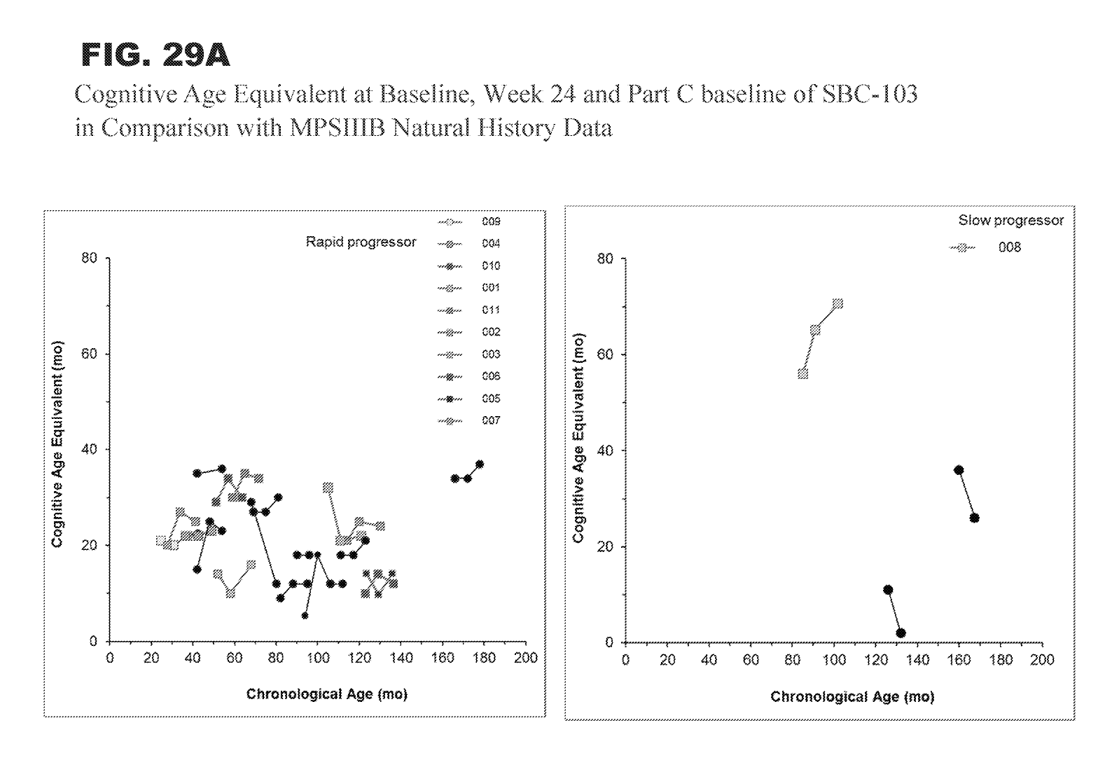

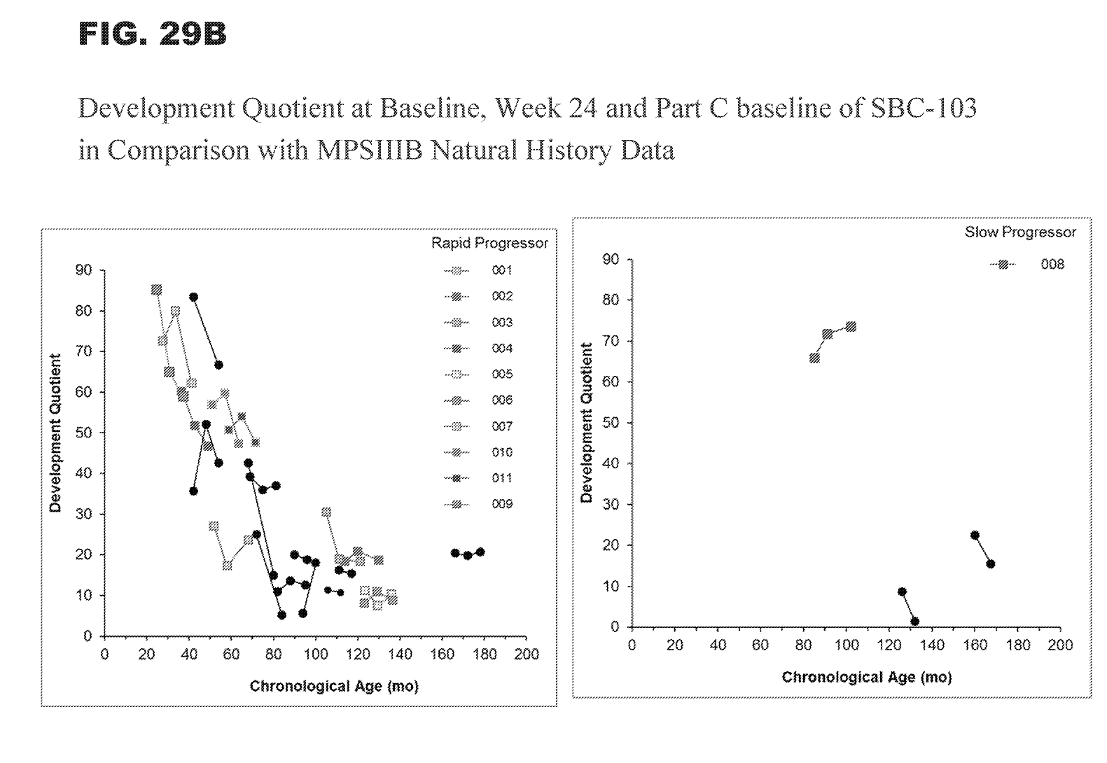

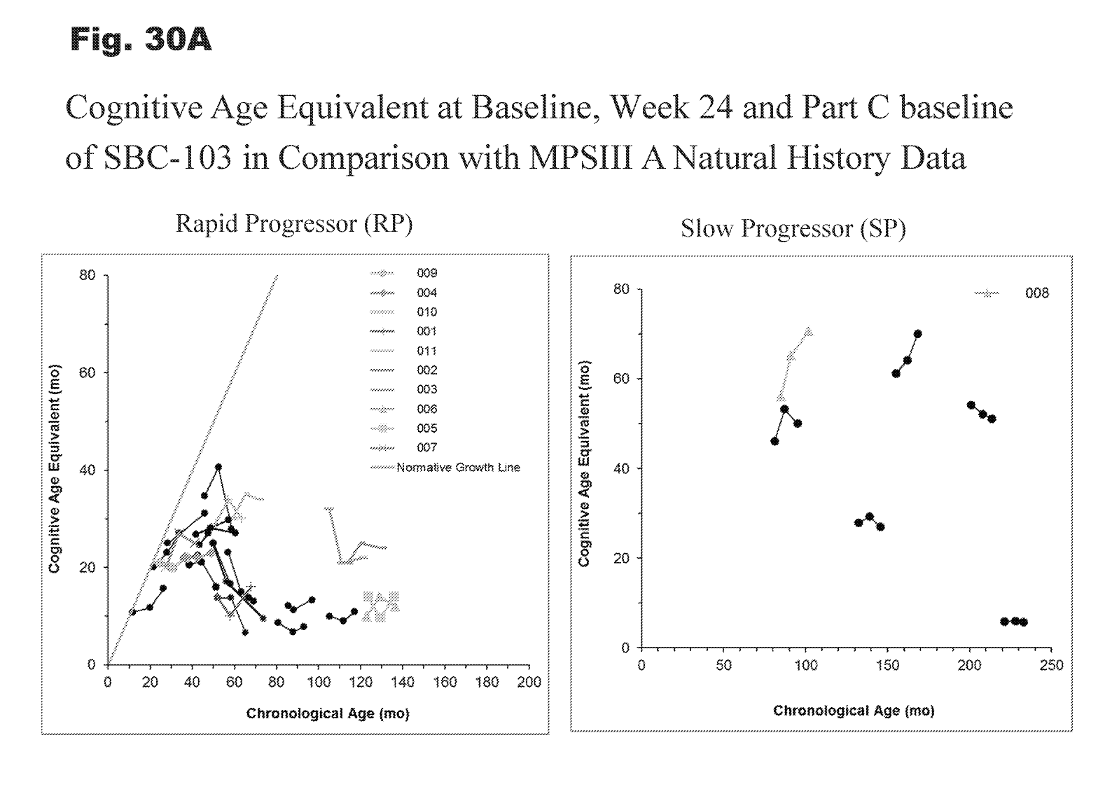

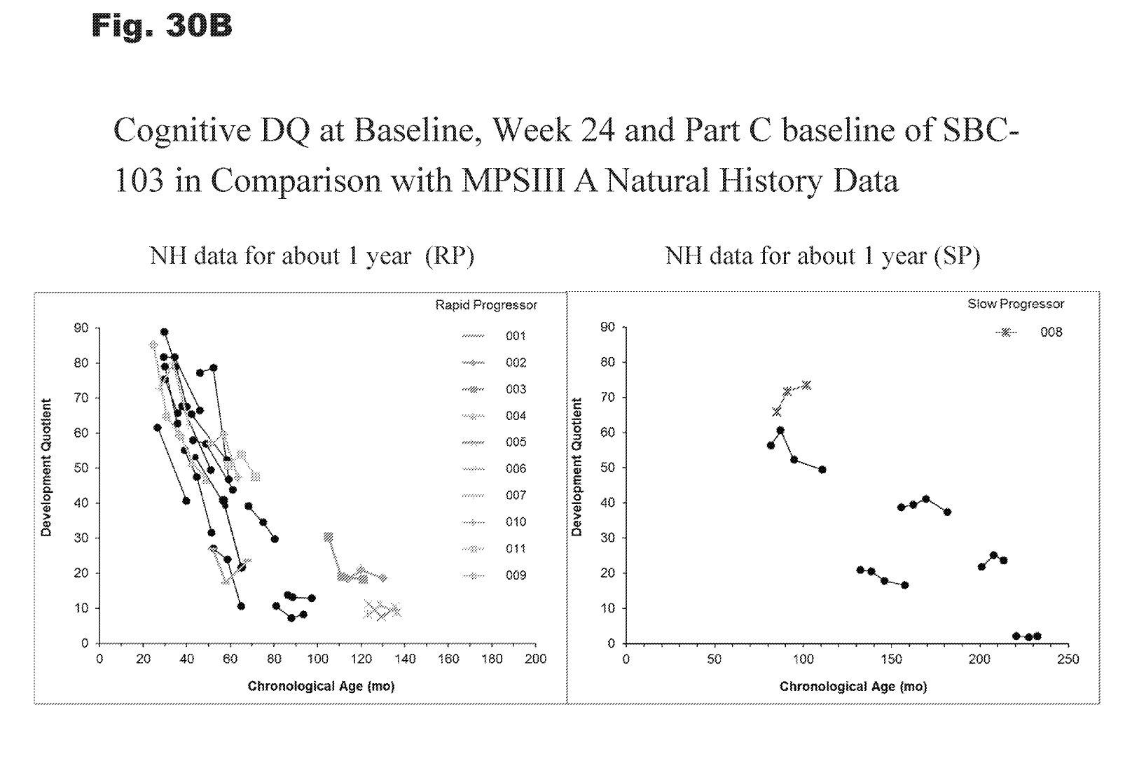

Cognitive AEq and DQ data for the individual clinical participants are plotted against chronological age in comparison with MPSIIIB Natural History in FIG. 29A and FIG. 29B respectively. Cognitive AEq and DQ data for the individual clinical participants are plotted against chronological age in comparison with MPSIIIA Natural History in FIG. 30A and FIG. 30B, respectively. Shaprio et al., J. Ped. 2016, 170:278-87.

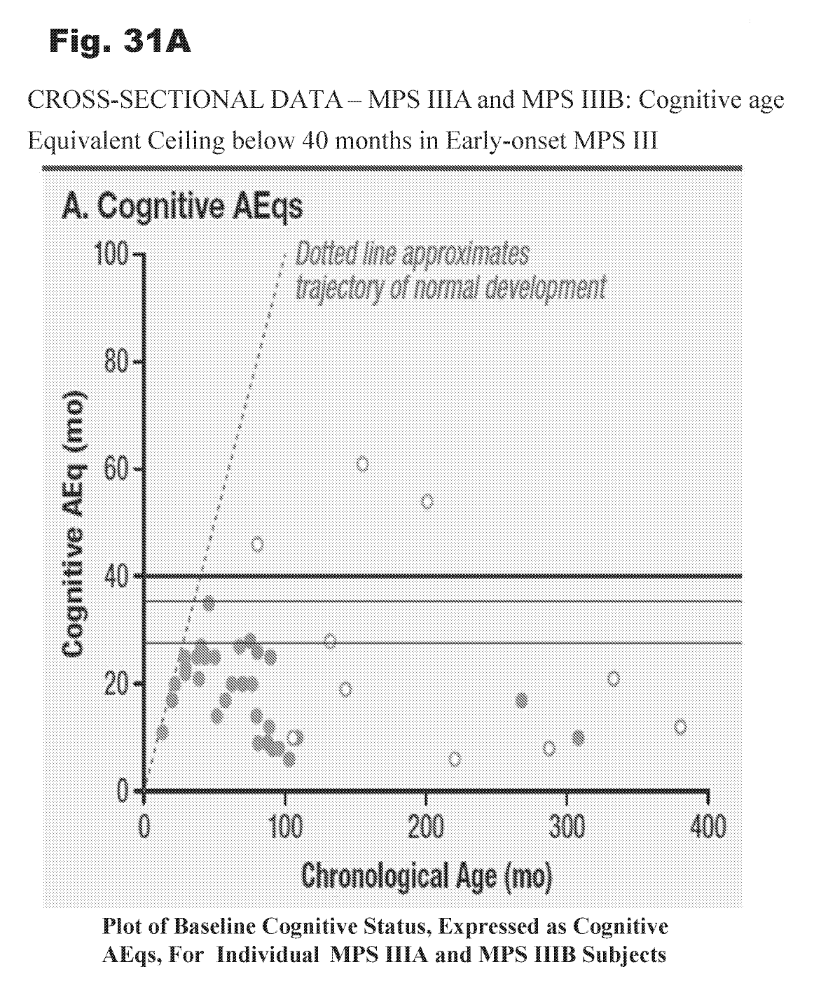

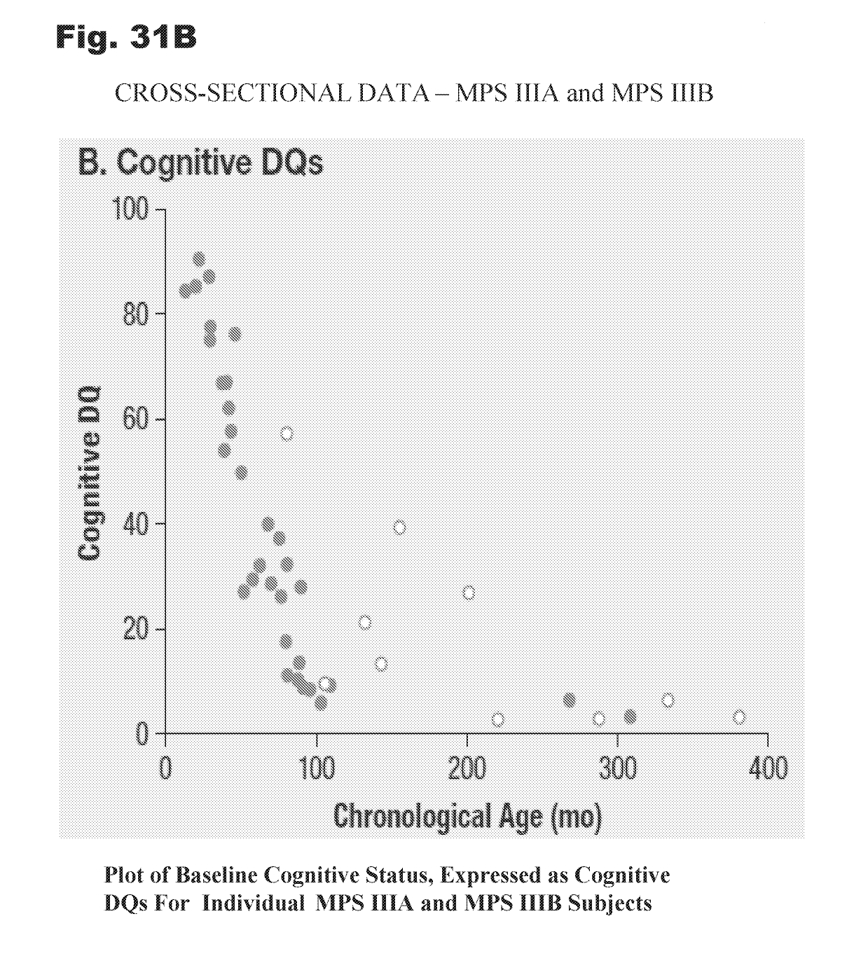

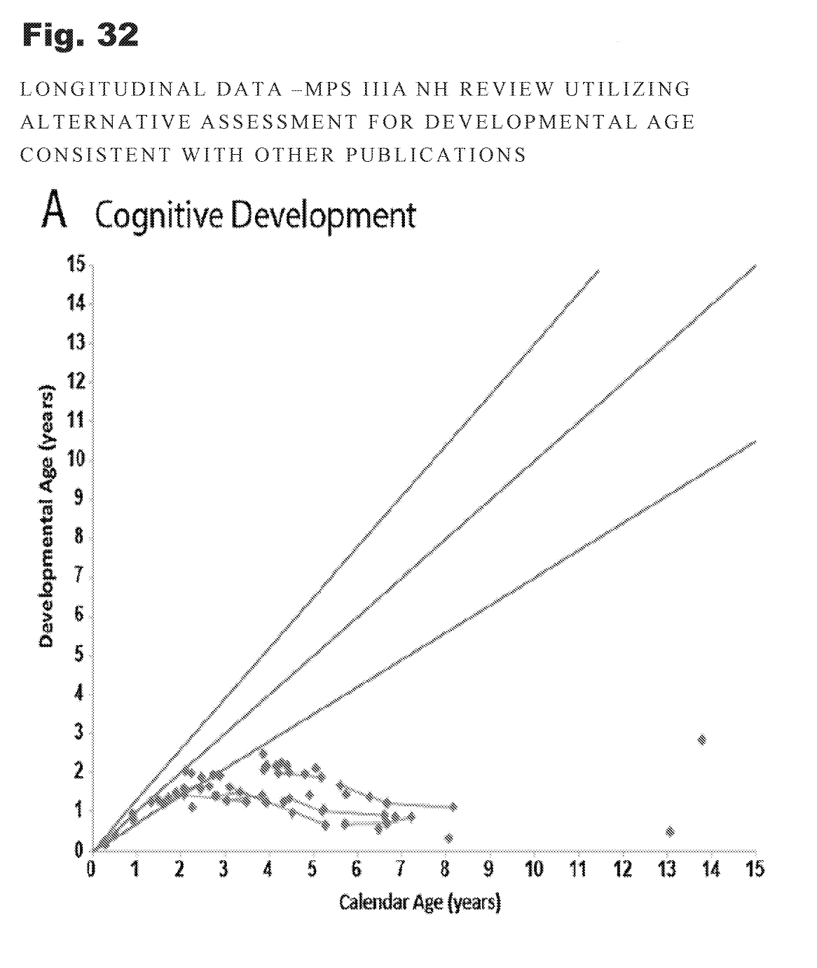

In connection with the neurocognitive assessments of the clinical participants, cross-sectional analysis of baseline data from the two independent natural history studies was reviewed. Cross-sectional analysis of cognitive AEq (FIG. 31A) and DQ (FIG. 31B) as related to chronological age using data reported in independent MPSIIIA and MPSIIIB natural history (NH) studies suggests that trajectories of adaptive behavioral and cognitive functions are very similar among MPS IIIA and MPS IIIB Rapid Progressors (early onset, diagnosed <6 years), that younger age of diagnosis is a surrogate for disease severity, and that Rapid Progressors are unlikely to achieve AEqs>40 mo in a naturalistic setting. The correlation of adaptive behavioral and cognitive functions suggests cross-validity of these independent measurements. FIG. 32 shows developmental age plotted against chronological age for 46 individual subjects in an MPSIIIA natural history study, using an alternative assessment tool, Mullen Scales of Early Learning (Buhrman et al., J Inherit. Metab. Dis. 2014 37(3):431-7), to calculate developmental age. Cognitive function was the weakest area of development, and analysis indicated a rapid decline in cognitive skills by 36 months of age.

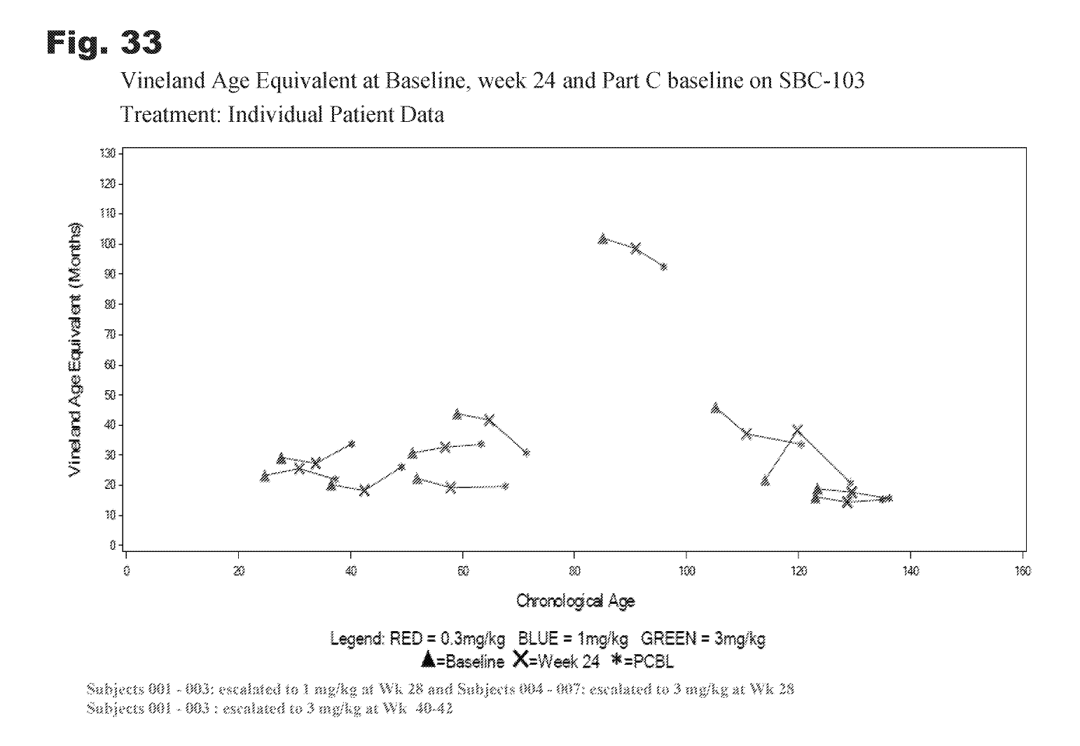

Vineland AEq (FIG. 33) assessed at three time points (baseline, week 24, and Part C baseline) for the individual clinical participants are plotted against chronological age. Table 13 shows data for individual subjects.

TABLE 13
Vineland Adaptive Behavior (+/− motor) AEq at Baseline, week 24
and Part C baseline of SBC-103 Treatment: Individual Patient Data

| | | | VABS AEq | |
|---|---|---|---|---|
| | | | (− motor) | |
| | Age at | Initial | (+ motor) | Change in |
| | Baseline | dose | BL/wk24/ | AE at |
| Sub # | (Months) | (mg/kg) | Part C BL | Part C BL |
| 004 | 36.5 | 1 | 18.6/16.6/22.7 | +4.1 |
| | | | 20.3/18.3/26.1 | +5.8 |
| 007 | 27.6 | 1 | 29.6/26.4/33.8 | +4.4 |
| | | | 29.2/27.3/33.8 | +4.6 |
| 009 | 24.7 | 3 | 23.2/25.1/22.8 | −0.4 |
| | | | 23.2/25.6/22.0 | −1.2 |
| 010 | 51 | 3 | 27.4/32.3/31.6 | +4.2 |
| | | | 30.8/32.6/33.7 | +2.9 |
| 011 | 59 | 3 | 47.2/43.8/30.9 | −16.3 |
| | | | 43.8/41.7/30.8 | −13 |
| 001 | 51.9 | 0.3 | 19.3/15.1/15.9/15.2 | −4.1 |
| | | | 22.3/19.3/21.0/19.7 | −2.6 |
| 002 | 114 | 0.3 | 18.1/34.3/13.2/18.7 | +0.6 |
| | | | 21.8/38.3/16.9/20.8 | −1 |
| 003 | 105.1 | 0.3 | 49.2/37.4/30.8/34.1 | −15.1 |
| | | | 45.9/37.1/32.1/33.6 | +12.3 |
| 005 | 123.4 | 1 | 16.3/14.0/11.9 | −4.4 |
| | | | 18.9/17.8/15.7 | −3.2 |
| 006 | 123 | 1 | 15.0/13.1/11.9 | −3.1 |
| | | | 16.1/14.5/15.3 | −0.8 |
| 008 | 85 | 3 | 83.7/78.2/100.7 | +17 |
| | | | 102.0/98.5/92.5 | −9.5 |

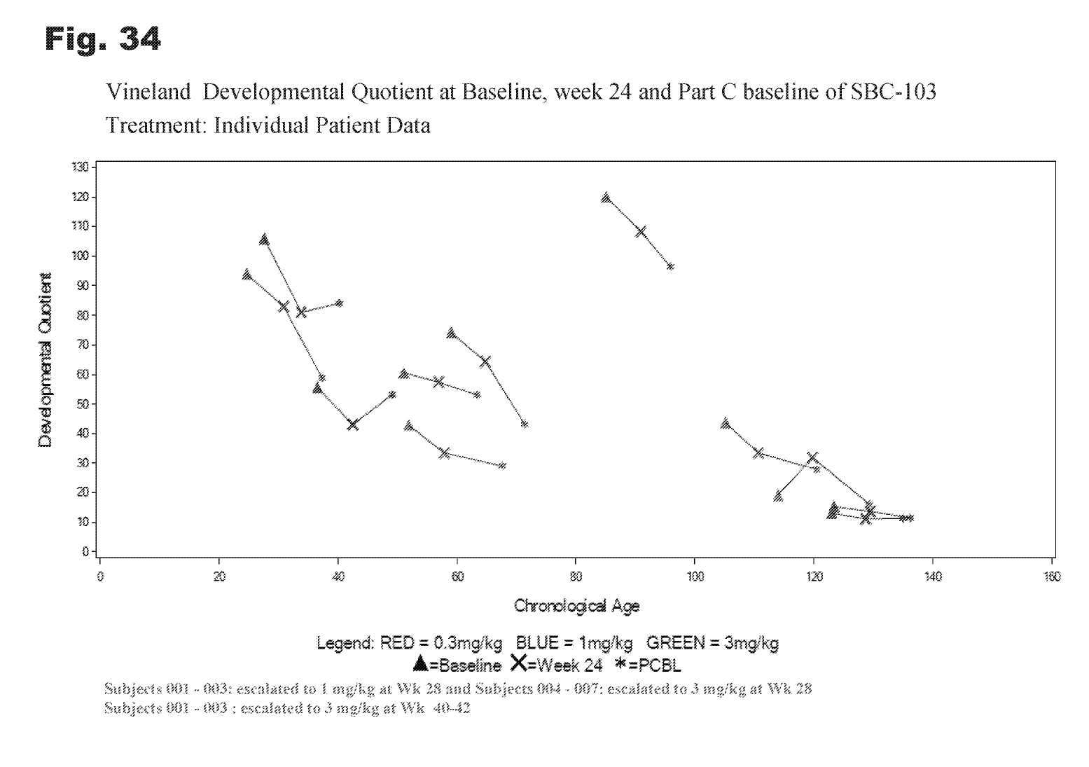

Vineland DQ (FIG. 34) assessed at three time points (baseline, week 24, and Part C baseline) for the individual clinical participants are plotted against chronological age. Table 14 shows data for individual subjects.

TABLE 14
Vineland Adaptive Behavior (+/− motor) DQ at Baseline, week 24 and
Part C baseline of SBC-103 Treatment: Individual Patient Data

| | | | VABS DQ | |
|---|---|---|---|---|
| | | | (− motor) | |
| | Age at | | (+ motor) | Change in |
| | Baseline | Initial dose | BL/wk24/Part C | DQ at |
| Sub # | (Months) | (mg/kg) | BL | Part C BL |
| 004 | 36.5 | 1 | 50.8/39.0/46.1 | −4.7 |
| | | | 55.6/43.1/53.2 | −2.4 |
| 007 | 27.6 | 1 | 107.3/78.3/84.0 | −23.3 |
| | | | 105.9/81.0/84.1 | −21.8 |
| 009 | 24.7 | 3 | 94.2/81.4/61.0 | −33.2 |
| | | | 94.0/83.0/58.8 | −35.2 |
| 010 | 51 | 3 | 53.8/56.8/49.8 | −4 |
| | | | 60.5/57.4/53.1 | −7.4 |
| 011 | 59 | 3 | 80.0/67.6/43.3 | −36.7 |
| | | | 74.2/64.4/43.1 | −31.1 |
| 001 | 51.9 | 0.3 | 37.3/26.1/24.7/22.5 | −14.8 |
| | | | 42.9/33.4/32.7/29.1 | −13.8 |
| 002 | 114 | 0.3 | 15.9/28.7/10.5/14.4 | −1.5 |
| | | | 19.2/31.9/13.4/16.1 | −3.1 |
| 003 | 105.1 | 0.3 | 46.8/33.8/26.3/28.3 | −18.5 |
| | | | 43.7/33.5/27.4/27.9 | −15.8 |
| 005 | 123.4 | 1 | 13.2/10.8/8.7 | −4.5 |
| | | | 15.3/13.7/11.5 | −3.8 |
| 006 | 123 | 1 | 12.2/10.2/8.8 | −3.4 |
| | | | 13.1/11.2/11.3 | −1.8 |
| 008 | 85 | 3 | 98.4/86.0/105.0 | +6.6 |
| | | | 120.0/108.4/96.5 | −23.5 |

### Discussion

The results show that intravenous administration of SBC-103 was well tolerated and biologically active in MPS IIIB patients. Preliminary evidence of disease stabilization was suggested by changes in brain structures (cortical gray matter volume, GMV) and neurocognitive assessments (AEq/DQ) in some patients in the context of available Sanfilippo syndrome (MPS III) natural history data.

Pharmacokinetic (PK) evaluation showed that PK exposure increased more than dose proportionally at Week 24 after multiple dose administration, and that HS reduction in CSF was linearly correlated with SBC-103 PK exposure increases, with moderate correlation coefficient.

Pharmacodynamic (PD) evaluation showed that, at Part C baseline, total HS percentage change in CSF from baseline, reported as mean (SD) was −2.10 (12.59), −4.15 (4.8) and −15.58 (4.84) for initial dose assignment of 0.3 mg/kg, 1 mg/kg, and 3 mg/kg groups, respectively (overall, −7.74 [9.22]); additionally, total HS percentage change in serum from baseline, reported as mean (SD)) was −59.32 (8.56), −56.67 (12.6) and −53.28 (7) for initial dose assignment of 0.3 mg/kg, 1 mg/kg, and 3 mg/kg groups, respectively (overall, −56.16 [9.13]).

Reduction of heparan sulfate in the CSF, PK/PD analysis, including observed changes in cortical gray matter volume and in various neurocognitive markers following administration of recombinant human NaGlu indicate that the recombinant human NaGlu penetrated the blood brain barrier.

Each example in the above specification is provided by way of explanation of the invention, not limitation of the invention. In fact, it will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention cover such modifications, combinations, additions, deletions, and variations.

All publications, patents, patent applications, interne sites, and accession numbers/database sequences (including both polynucleotide and polypeptide sequences) cited herein are hereby incorporated by reference in their entirety for all purposes to the same extent as if each individual publication, patent, patent application, interne site, or accession number/database sequence were specifically and individually indicated to be so incorporated by reference.

What is claimed is:

1. A method of treating a subject suffering from N-acetyl-alpha-D-glucosaminidase (NaGlu) deficiency, the method comprising periodically intravenously administering to the subject a therapeutically effective amount of recombinant human N-acetyl-alpha-D-glucosaminidase (rhNaGlu), wherein a dose of rhNaGlu is administered by intravenous infusion to the subject at least once every two weeks for a period of at least 24 weeks.

2.-6. (canceled)

7. The method of claim 1, wherein a dose of rhNaGlu of about 0.3 mg/Kg to about 10 mg/Kg is administered to the subject.

8. (canceled)

9. The method of claim 1, wherein a dose of rhNaGlu of about 5 mg/Kg to about 10 mg/Kg is administered to the subject.

10. The method of claim 1, wherein a dose of rhNaGlu of at least about 10 mg/Kg is administered to the subject.

11.-17. (canceled)

18. The method of claim 1, wherein a dose of rhNaGlu is administered to the subject periodically for a first period, followed by a dose escalation for a second period.

19. The method of claim 18, wherein the dose escalation comprises a higher dose of rhNaGlu, or more frequent administration of the dose of rhNaGlu, or both.

20.-27. (canceled)

28. The method of claim 1, wherein rhNaGlu is administered in an amount and for a period sufficient to:
(a) slow a decline in, stabilize, or increase the cortical gray matter volume of the brain of the subject in comparison to a baseline level of cortical gray matter volume of the subject prior to initiation or escalation of treatment;
(b) slow a decline in, stabilize, or improve a neurocognitive or behavioral indicator of the subject in comparison to a baseline level of the neurocognitive indicator of the subject prior to initiation or escalation of treatment; and/or
(c) decrease the total heparan sulfate (HS) level in the cerebral spinal fluid (CSF) of the subject in comparison to a baseline level of the subject prior to initiation or escalation of treatment.

29. (canceled)

30. The method of claim 28, wherein rhNaGlu is administered to the subject for a period of at least 52 weeks, and wherein the cortical gray matter volume of the subject is at least 90% of the baseline level.

31.-32. (canceled)

33. The method of claim 28, wherein rhNaGlu is administered in an amount and for a period sufficient to slow a decline in, stabilize, or increase the cortical gray matter volume of the brain of the subject in comparison to the cortical gray matter volume of an untreated age-matched patient suffering from mucopolysaccharidosis IIIA (MPSIIIA) or mucopolysaccharidosis IIIB (MPSIIIB), or in comparison to the average cortical gray matter volume of a plurality of untreated age-matched patients suffering from mucopolysaccharidosis IIIA (MPSIIIA) or mucopolysaccharidosis TIM (MPSIIIB).

34.-37. (canceled)

38. The method of claim 28, further comprising determining the cortical gray matter volume of the subject.

39.-42. (canceled)

43. The method of claim 28, wherein rhNaGlu is administered to the subject for a period of at least 24 weeks, and wherein the cognitive age equivalence (AEq) of the subject increases, is unchanged, or decreases by no more than 3 months in comparison to the baseline level; and/or the cognitive development quotient (DQ) of the subject decreases by no more than 15 points from the baseline level.

44.-55. (canceled)

56. The method of claim 28, wherein
(i) the chronological age of the subject at baseline is less than or equal to 36 months, and wherein the behavioral age equivalence (AEq) of the subject increases, is unchanged, or decreases by no more than 3 months in comparison to the baseline level; or
(ii) the chronological age of the subject at baseline is greater than 36 months, and wherein the behavioral age equivalence (AEq) of the subject increases, is unchanged, or decreases by no more than 15 months in comparison to the baseline level.

57.-60. (canceled)

61. The method of claim 28, wherein
(i) the chronological age of the subject at baseline is less than or equal to 60 months, and wherein the behavioral development quotient (DQ) of the subject decreases by no more than 40 points from the baseline level; or
(ii) the chronological age of the subject at baseline is greater than 60 months, and wherein the behavioral development quotient (DQ) of the subject decreases by no more than 20 points from the baseline level.

62.-65. (canceled)

66. The method of claim 28, wherein rhNaGlu is administered in an amount and for a period sufficient to slow a decline in, stabilize, or improve a neurocognitive or behavioral indicator of the subject in comparison to the neurocognitive or behavioral indicator of an untreated age-matched patient suffering from mucopolysaccharidosis IIIA (MPSIIIA) or mucopolysaccharidosis IIIB (MPSIIIB), or in comparison to the average value of a neurocognitive or behavioral indicator of a plurality of untreated age-matched patients suffering from mucopolysaccharidosis IIIA (MPSIIIA) or mucopolysaccharidosis IIIB (MPSIIIB).

67.-70. (canceled)

71. The method of claim 28, further comprising assessing the neurocognitive or behavioral indicator of the subject.

72.-74. (canceled)

75. The method of claim 28, wherein rhNaGlu is administered to the subject for a period of at least 24 weeks, and wherein total HS level in the CSF of the subject is less than 75% of the baseline level.

76. (canceled)

77. The method of claim 28, further comprising determining the total HS level in the CSF of the subject.

78. A method for treating a subject suffering from NaGlu deficiency by
(i) slowing a decline in, stabilizing, or increasing the cortical gray matter volume of the brain of the subject
(ii) arresting or reversing the progression of mucopolysaccharidosis IIIB (MPS IIIB) in the subject
(iii) slowing, arresting, or reversing neurocognitive decline in a subject
(iv) improving a neurocognitive or behavioral indicator for the subject and/or
(v) reducing the total heparan sulfate (HS) level in cerebral spinal fluid (CSF) of a subject, the method comprising periodically intravenously administering to the subject a therapeutically effective amount of rhNaGlu, wherein a dose of rhNaGlu is administered by intravenous infusion to the subject at least once every two weeks for a period of at least 24 weeks.

79.-90. (canceled)

91. The method of claim 1, wherein the cognitive developmental quotient (DQ) of the subject at baseline is over 50.

92.-109. (canceled)

110. A pharmaceutical composition comprising:
recombinant human NaGlu; and
a pharmaceutical carrier;
wherein the recombinant human NaGlu is present in an amount sufficient to
decrease the level of heparan sulfate in the cerebral spinal fluid of a subject;
stabilize or increase the cortical gray matter volume of the brain of a subject; and/or
stabilize or improve a neurocognitive indicator of the subject;
in comparison to a baseline level of the subject prior to initiation or escalation of treatment; and
wherein the composition is formulated for intravenous infusion.

111. (canceled)


SEQUENCE LISTING (5 sequences)

SEQ ID NO: 1 - protein, 743 aa, Human
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 2 - DNA, 2234 bp, Human
atggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcagccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactctttcgtgtggtgggactgggcccggtgggagcgagagatagactggatggcgctgaatggcatcaacctggcactggcatggagcggccaggaggccatctggcagcgggtgtacctggccttgggcctgacccaggcagagatcaatgagttctttactggtcctgccttcttggcatgggggcgaatgggcaacctgcacacctgggatggccccctgcccccctcctggcacatcaagcagctttatctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcttgcgagagctgatcaaagagtttggcacagaccacatctatggggccgacactttcaatgagatgcagccaccttcctcagagccctcctatcttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccaaggccagcccttcatctggtgcatgctgcacaactttgggggaaatcatggtctttttggagccttggaggccgtgaacggaggcccagaagctgcccgcctcttccccaactccacaatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcatgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtattatgaggaggcaagaagcgcctatctgagcaaggagctggcctccttgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattacccccgctgggtggccggctcttggtgatt

SEQ ID NO: 3 - DNA, 1132 bp, Chicken
gttaagtcctcagacttggcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaataaaagtttgttataaagcattcacacccataaaaagatagatttaaatattccaactataggaaagaaagtgtgtctgctcttcactctagtctcagttggctccttcacatgcacgcttctttatttctcctattttgtcaagaaaataataggtcaagtcttgttctcatttatgtcctgtctagcgtggctcagatgcacattgtacatacaagaaggatcaaatgaaacagacttctggtctgttactacaaccatagtaataagcacactaactaataattgctaattatgttttccatctccaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcagactatatattccccagggctcagccagtgtctgt

SEQ ID NO: 4 - DNA, 10256 bp, Artificial Sequence
  pSIN-OV-1.1-I-rhNaGlu
ggccgcaagaagaaagctgaaaaactctgtcccttccaacaagacccagagcactgtagtatcaggggtaaaatgaaaagtatgttatctgctgcatccagacttcataaaagctggagcttaattcagaaaaaaaatcagaaagaaattacactgtgagaacaggtgcaattcacttttcctttacacagagtaatactggtaactcatggatgaaggcttaagggaatgaaattggactcacagtactgagtcatcacactgaaaaatgcaacctgatacatcagcagaaggtttatgggggaaaaatgcagccttccaattaagccagatatctgtatgaccaagctgctccagaattagtcactcaaaatctctcagattaaattatcaactgtcaccaaccattcctatgctgacaaggcaattgcttgttctctgtgttcctgatactacaaggctcttcctgacttcctaaagatgcattataaaaatcttataattcacatttctccctaaactttgactcaatcatggtatgttggcaaatatggtatattactattcaaattgttttccttgtacccatatgtaatgggtcttgtgaatgtgctcttttgttcctttaatcataataaaaacatgtttaagcaaacacttttcacttgtagtatttgaaggtaccggatctcgagccgccttcaatgcccccaaaaccaatccccaggtttttaactctcccgattttccaagtaccatagcccgctgagagagcgccgcggtaatgggatcccaggaccccggggaatataagtctgagggggacgtaagcaacccttccttttgtaacagggacaacatagcccctatttccttcttagaaggagaggttttcccgcaataggtcttacacgcggacgaaatcacctttatgacggcttccatgcttgatccaccgggcgaccggaatcacgcagagcaaccggaatcacgcctggggtggaccgctcagtcgtcgggcttccttcccgtcttccaacgactctctgagttctcggtagggtatgttggccccctgcagtagggctccctccgacgccactcagcttctgccctcctaagccgcagccccctctactagggtcatcgtccgctccccgaataagcgagacggatgaggacaggatcgccacgccgcctgtggccgaccactattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtgttagacccgtctgttgccttcctaacaaggcacgatcataccacgatcataccaccttactcccaccaatcggcatgcacggtgctttttctctccttataaggcatgttgctaactcatcgttacataagcatgttgcaagactacaagagtattgcataagactacatttccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgtattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatccaaccagctatcacacggaatacaagaactcgcctacgctcttctttcgggctgcttataagcctcctgtaatttttttatattcctcgttaagtcctcagacttggcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaataaaagtttgttataaagcattcacacccataaaaagatagatttaaatattccaactataggaaagaaagtgtgtctgctcttcactctagtctcagttggctccttcacatgcacgcttctttatttctcctattttgtcaagaaaataataggtcaagtcttgttctcatttatgtcctgtctagcgtggctcagatgcacattgtacatacaagaaggatcaaatgaaacagacttctggtctgttactacaaccatagtaataagcacactaactaataattgctaattatgttttccatctccaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcagactatatattccccagggctcagccagtgtctgtacatacagctagaaagctgtattgcctttagcagtcaagctcgaaaggtaagcaactctctggaattaccttctctctatattagctcttacttgcacctaaactttaaaaaattaacaattattgtgctatgtgttgtatctttaagggtgaagtacctgcgtgataccccctataaaaacttctcacctgtgtatgcattctgcactattttattatgtgtaaaagctttgtgtttgttttcaggaggcttattctttgtgcttaaaatatgtttttaatttcagaacatcttatcctgtcgttcactatctgatatgctttgcagtttgcttgattaacttctagccctacagagtgcacagagagcaaaatcatggtgttcagtgaattctggggagttattttaatgtgaaaattctctagaagtttaattcctgcaaagtgcagctgctgatcactacacaagataaaaatgtggggggtgcataaacgtatattcttacaataatagatacatgtgaacttatatacagaaaagaaaatgagaaaaatgtgtgtgtgtatactcacacacgtggtcagtaaaaacttttgaggggtttaatacagaaaatccaatcctgaggccccagcactcagtacgcatataaagggctgggctctgaaggacttctgactttcacagattatataaatctcaggaaagcaactagattcatgctggctccaaaagctgtgctttatataagcacactggctatacaatagttgtacagttcagctctttataatagaaacagacagaacaagtataaatcttctattggtctatgtcatgaacaagaattcattcagtggctctgttttatagtaaacattgctattttatcatgtctgcatttctcttctgtctgaatgtcaccactaaaatttaactccacagaaagtttatactacagtacacatgcatatctttgagcaaagcaaaccatacctgaaagtgcaatagagcagaatatgaattacatgcgtgtctttctcctagactacatgaccccatataaattacattccttatctattctgccatcaccaaaacaaaggtaaaaatacttttgaagatctactcatagcaagtagtgtgcaacaaacagatatttctctacatttatttttagggaataaaaataagaaataaaatagtcagcaagcctctgctttctcatatatctgtccaaacctaaagtttactgaaatttgctctttgaatttccagttttgcaagcctatcagattgtgttttaatcagaggtactgaaaagtatcaatgaattctagctttcactgaacaaaaatatgtagaggcaactggcttctgggacagtttgctacccaaaagacaactgaatgcaaatacataaatagatttatgaatatggttttgaacatgcacatgagaggtggatatagcaacagacacattaccacagaattactttaaaactacttgttaacatttaattgcctaaaaactgctcgtaatttactgttgtagcctaccatagagtaccctgcatggtactatgtacagcattccatccttacattttcactgttctgctgtttgctctagacaactcagagttcaccatggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcagccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactctttcgtgtggtgggactgggcccggtgggagcgagagatagactggatggcgctgaatggcatcaacctggcactggcatggagcggccaggaggccatctggcagcgggtgtacctggccttgggcctgacccaggcagagatcaatgagttctttactggtcctgccttcttggcatgggggcgaatgggcaacctgcacacctgggatggccccctgcccccctcctggcacatcaagcagctttatctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcttgcgagagctgatcaaagagtttggcacagaccacatctatggggccgacactttcaatgagatgcagccaccttcctcagagccctcctatcttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccaaggccagcccttcatctggtgcatgctgcacaactttgggggaaatcatggtctttttggagccttggaggccgtgaacggaggcccagaagctgcccgcctcttccccaactccacaatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcatgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtattatgaggaggcaagaagcgcctatctgagcaaggagctggcctccttgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattacccccgctgggtggccggctcttggtgattcgaagc

SEQ ID NO: 5 - DNA, 2234 bp, Human
atggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactctttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattacccccgctgggtggccggctcttggtgata